(19) United States
(12) Patent Application Publication
(10) Pub. No.: US 2024/0190858 A1
(43) Pub. Date: Jun. 13, 2024

(54) CRYSTALLINE FORMS, PHARMACEUTICAL COMPOSITIONS AND METHODS OF USE THEREOF

(71) Applicant: Tango Therapeutics, Inc., Boston, MA (US)

(72) Inventors: Kimberly Jane Briggs, Framingham, MA (US); Kevin M. Cottrell, Arlington, MA (US); Sapna Makhija Garad, Malden, MA (US); Magnus Ronn, Melrose, MA (US)

(21) Appl. No.: 18/494,314

(22) Filed: Oct. 25, 2023

Related U.S. Application Data
(60) Provisional application No. 63/419,225, filed on Oct. 25, 2022.
(60) Provisional application No. 63/496,105, filed on Apr. 14, 2023.

(51) Int. Cl. C07D 417/14; A61K 9/20; A61K 9/28; A61P 35/00

(52) U.S. Cl. CPC C07D 417/14; A61K 9/2009; A61K 9/2013; A61K 9/2054; A61K 9/284; A61P 35/00; C07B 2200/13

(57) ABSTRACT

Crystalline forms and pharmaceutical compositions of a PRMT5 inhibitor of formula (I), methods of making the pharmaceutical compositions of the PRMT5 inhibitor of formula (I) and methods of using the PRMT5 inhibitor of formula (I) or crystalline solid forms and pharmaceutically acceptable compositions thereof.

### CROSS-REFERENCE TO RELATED APPLICATIONS

This application claims the benefit of and priority to U.S. Provisional Application No. 63/419,225, filed Oct. 25, 2022 and U.S. Provisional Application No. 63/496,105, filed Apr. 14, 2023, which are incorporated herein by reference in their entireties.

### BACKGROUND

Development of pharmaceutical compositions comprising one or more novel active ingredients requires a variety of considerations, such as route of administration (e.g., enteral, parenteral, topical, etc.), dosage form (e.g., solid—tablet, capsule, etc.; liquid—solution, suspension, syrup, etc.), strength of active ingredient(s) (e.g., 1 mg-1,000 mg), non-therapeutic component(s) (e.g., excipients) and their respective amounts, and each of these considerations may involve additional considerations such as stability, degradation, sensitivity to light, solubility, taste if administered enterally, palatability, pH, skin irritability, microbial growth, etc. Advancing a novel active ingredient (e.g., a PRMT5 inhibitor) through rigorous regulatory entities requires discovering and developing a pharmaceutical composition that addresses these, or other, considerations.

Accordingly, there is a need for pharmaceutical compositions comprising compounds (e.g., PRMT5 inhibitors) that exhibit desirable properties treat diseases or disorders (e.g., cancers) in human patients.

### SUMMARY

In some embodiments, provided is a crystalline form of N-(6-amino-5-ethylpyridin-3-yl)-2-((2R,5S)-5-methyl-2-(2-(1-methylpiperidin-4-yl)benzo[d]thiazol-5-yl)piperidin-1-yl)-2-oxoacetamide (a compound of formula (I))

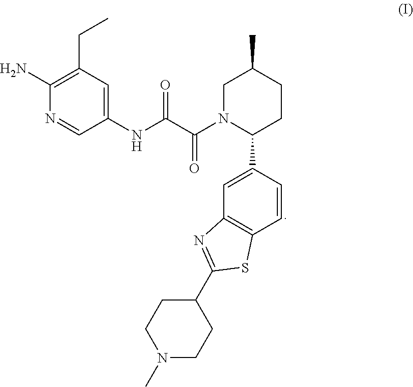 [SMILES: CCc1cc(NC(=O)C(=O)N2C[C@@H](C)CC[C@@H]2c2ccc3sc(C4CCN(C)CC4)nc3c2)cnc1N]

In some embodiments, provided is a crystalline form of N-(6-amino-5-ethylpyridin-3-yl)-2-((2R,5S)-5-methyl-2-(2-(1-methylpiperidin-4-yl)benzo[d]thiazol-5-yl)piperidin-1-yl)-2-oxoacetamide (a compound of formula (I))

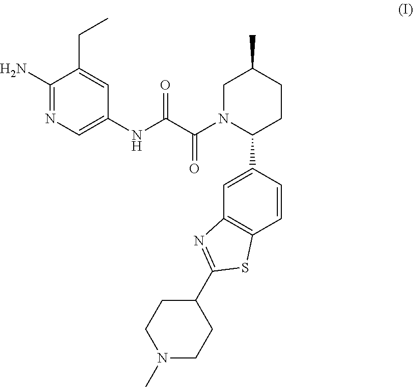 [SMILES: CCc1cc(NC(=O)C(=O)N2C[C@@H](C)CC[C@@H]2c2ccc3sc(C4CCN(C)CC4)nc3c2)cnc1N]

wherein the X-ray powder diffraction (XRPD) pattern of the crystalline form comprises one or more peaks at 2θ angles selected from 9.6±0.2, 16.8±0.2, 19.3±0.2, 19.4±0.2, 20.9±0.2, 23.7±0.2 and 24.5±0.2 degrees (Form A).

In some embodiments, provided are pharmaceutical compositions comprising a compound of formula (I), or a pharmaceutically acceptable salt thereof, and at least one pharmaceutically acceptable excipient.

In certain embodiments, the pharmaceutical compositions comprise crystalline form A of the compound of Formula (I).

In some embodiments, provided is a pharmaceutical composition comprising:
(a) a compound of formula (I)

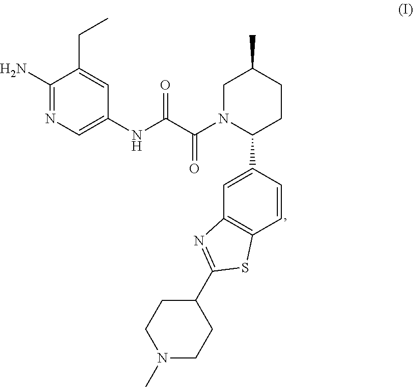 [SMILES: CCc1cc(NC(=O)C(=O)N2C[C@@H](C)CC[C@@H]2c2ccc3sc(C4CCN(C)CC4)nc3c2)cnc1N]

or a pharmaceutically acceptable salt thereof;
(b) a filler (e.g., microcrystalline cellulose);
(c) a glidant (e.g., colloidal silicon dioxide);
(d) a disintegrant (e.g., croscarmellose sodium); and
(e) a lubricant (e.g., magnesium stearate).

In some embodiments, the composition comprises a crystalline form of the compound of formula (I) described herein (e.g., Form A).

In some embodiments, provided is a dosage form containing a pharmaceutical composition as described herein.

In some embodiments, provided is a method for treating an MTAP-deficient and/or an MTA-accumulating disease in a subject in need thereof comprising administering to the subject a therapeutically effective amount of a crystalline form as described herein (e.g., Form A of a compound of formula (I).

In some embodiments, provided is a method for treating an MTAP-deficient and/or an MTA-accumulating disease in a subject in need thereof comprising administering to the subject a pharmaceutical composition as described herein, containing a therapeutically effective amount of the compound of Formula (I).

### BRIEF DESCRIPTION OF THE DRAWINGS

FIG. 1 is an exemplary X-ray powder diffraction pattern of crystalline Form A of the compound of Formula (I).

FIG. 2A shows an exemplary thermogravimetric analysis (TGA) thermogram of the compound of Formula (I).

FIG. 2B shows a differential scanning calorimetry (DSC) thermogram for crystalline Form A the compound of Formula (I), under a heat-cool-heat protocol, showing a melt peak at about 145.8° C.

FIG. 3A shows the change of water content (solid curve) and relative humidity (hashed curve) as a function of time in an exemplary DVS experiment for the compound of Formula (I) (Crystalline form A).

FIG. 3B shows the change of water content as a function of relative humidity in an exemplary DVS experiment for the compound of Formula (I) (Crystalline form A).

FIG. 4A shows the antiproliferative activity (viability at different concentrations) of the compound of Formula (I) in an MTAP-isogenic cell line pair engineered by CRISPR-mediated MTAP gene knockout of the HAP1 chronic myeloid leukemia cell line.

FIG. 4B shows the antiproliferative activity (viability at different concentrations) of the compound of Formula (I) in an MTAP-isogenic cell line pair engineered by CRISPR-mediated MTAP gene knockout of the HCT116 colorectal cancer cell line.

FIG. 4C shows the antiproliferative activity (viability at different concentrations) of the compound of Formula (I) in an MTAP-isogenic cell line pair engineered by reconstituting exogenous MTAP in an endogenous MTAP-deleted LU99 non-small cell lung cancer cell line.

FIG. 4D shows the antiproliferative activity (viability at different concentrations) of the compound of Formula (I) in an MTAP-isogenic cell line pair engineered by reconstituting exogenous MTAP in an endogenous MTAP-deleted LN18 glioblastoma cell line.

FIG. 5A shows the pharmacodynamic activity of the compound of Formula (I) against PRMT5, by showing a graph of a normalized single SDMA-modified protein level at various concentrations of PRMT5 inhibitor of Formula (I) in a HAP 1 MTAP-isogenic cell line pair. Normalized to a DMSO control for each cell line and represented as mean±SD.

FIG. 5B shows the pharmacodynamic activity of the compound of Formula (I) against Type I PRMTs, by showing a graph of a normalized single ADMA-modified protein level at various concentrations of PRMT5 inhibitor of Formula (I) in a HAP 1 MTAP-isogenic cell line pair. Normalized to a DMSO control for each cell line and represented as mean±SD.

FIG. 5C shows a dendrogram of biochemical selectivity of the compound of formula (I) for PRMT5 in a histone methyltransferase panel.

FIG. 6 shows the pharmacokinetic profile in cynomolgus monkey of the compound of Formula (I), depicting the free plasma exposures following 3 mg/kg oral gavage of the compound of Formula (I). The GI₅₀ for WT and MTAP-null cells is indicated by dotted lines.

FIG. 7 shows the results of a 7-day PK/PD study using a LU99 (lung giant cell carcinoma) MTAP-null xenograft model. The compound of Formula (I) was dosed as indicated, and PK and tumor samples were harvested at the indicated timepoints; the SDMA-modified protein levels for each dose as a proportion of vehicle are shown. N=4 tumors per group, and data are presented as mean±SEM.

FIG. 8A shows the dose-dependent antitumor activity of the compound of Formula (I) against an LN18 MTAP-null cell line-derived xenograft model. Data are presented as mean±SEM.

FIG. 8B shows the dose-dependent antitumor activity of the compound of Formula (I) against an OCI-LY19 MTAP-null cell line-derived xenograft model. Data are presented as mean±SEM.

FIG. 8C shows the dose-dependent antitumor activity of the compound of Formula (I) against an MTAP-null mesothelioma patient-derived xenograft model. Data are presented as mean±SEM.

FIG. 8D shows the dose-dependent antitumor activity of the compound of Formula (I) against an MTAP-null bladder cancer patient-derived xenograft model. Data are presented as mean±SEM.

FIG. 8E shows the dose-dependent antitumor activity of the compound of Formula (I) against an MTAP-null cholangiocarcinoma patient-derived xenograft model. Data are presented as mean±SEM.

FIG. 8F shows the dose-dependent antitumor activity of the compound of Formula (I) against an MTAP-null NSCLC (squamous) patient-derived xenograft model. Data are presented as mean±SEM.

FIG. 8G shows the dose-dependent antitumor activity of the compound of Formula (I) (40 mg/kg BID and 100 mg/kg QD) against a LU-99 NSCLC MTAP-null cell line-derived xenograft model. The dotted line is the regression line, which is defined as final mean tumor volume that is 30% less than the initial mean tumor volume. Data are presented as mean±SEM.

FIG. 8H shows the dose-dependent antitumor activity of 30 mg/kg BID and 60 mg/kg BID of the compound of Formula (I) against a LU-99 NSCLC MTAP-null cell line-derived xenograft model. The dotted line is the regression line, which is defined as final mean tumor volume that is 30% less than initial mean tumor volume. Data are presented as mean±SEM.

FIG. 9A shows the antitumor activity of the compound of Formula (I) in patient-derived xenograft models obtained from bladder cancer, cholangiocarcinoma, mesothelioma, non-small cell lung cancer (e.g., adenocarcinoma and squamous cell carcinoma), and pancreatic cancer (e.g., pancreatic ductal adenocarcinoma). If the final mean tumor volume for the PRMT5 inhibitor-treated mice was greater than the initial mean tumor volume, the data are presented in relation to vehicle-treated tumors (greater than 0). If the final mean tumor volume for the PRMT5 inhibitor-treated mice was less than the initial mean tumor volume, then the data are presented relative to mean tumor volume (less than 0).

FIG. 9B shows an updated representation of the data from 9A with an additional 3 datapoints for cholangiocarcinoma (1), bladder (1) and lung (1) cancer xenografts. The data for adenocarcinoma and squamous carcinoma of the lung is consolidated in a single category. The graph represents final tumor volume as a percentage of initial tumor volume—the 100 line represents no change in tumor volume, values between 0-100% of initial tumor represent reductions in tumor volume, and values above 100 represent increases in tumor volume.

FIG. 10A shows the ability of compound of Formula (I) to overcome resistance to the MTA-cooperative PRMT5 inhibitor in a diffuse large B cell lymphoma cell line-derived xenograft model. Data are presented as mean±SEM.

FIG. 10B shows the enlarged portion of FIG. 10A with broken y-axis to highlight the region of interest.

FIG. 11 shows waterfall plots of 180 cancer cell lines representing multiple cancer lineages including NSCLC, PDAC, bladder, CNS, and heme malignancies, which were profiled with PRMT5 inhibitor of Formula (I) in a 7-day CellTiter-Glo assay. The maximum effect at a concentration equal to 10× the HAP1 MTAP-null GI₅₀ is reported and the cell lines are fill-coded by MTAP status.

FIG. 12 shows the antitumor activity of the compound of Formula (I), at 30 mg/kg BID, alone or in combination with 1 mg/kg QD osimertinib, against an NCI-H1650 NSCLC MTAP-null cell line-derived xenograft model. Data are presented as mean±SEM.

FIG. 13 shows the antitumor activity of the compound of Formula (I), at 30 and 60 mg/kg BID alone and at 30 mg/kg BID in combination with 10 mg/kg BID AGI-41998, against an NCI-H838 NSCLC MTAP-null cell line-derived xenograft model. Data are presented as mean±SEM.

### DETAILED DESCRIPTION

As generally described herein, the present disclosure provides pharmaceutical compositions containing a PRMT5 inhibitor (e.g., an MTA-uncompetitive PRMT5 inhibitor) e.g., a compound of formula (I):

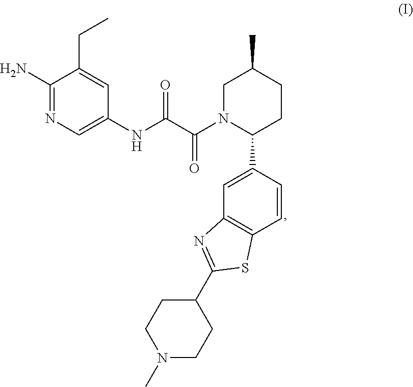 [SMILES: CCc1cc(NC(=O)C(=O)N2C[C@@H](C)CC[C@@H]2c2ccc3sc(C4CCN(C)CC4)nc3c2)cnc1N]

and crystalline forms thereof, methods of making the pharmaceutical compositions, and methods of using the pharmaceutical compositions to treat medical conditions, diseases, and disorders e.g., proliferation diseases such as cancers.

### Definitions

As used in the present disclosure, the following words and phrases are generally intended to have the meanings as set forth below unless expressly indicated otherwise or the context in which they are used indicates otherwise.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. Where elements are presented as lists, e.g., in Markush group format, each subgroup of the elements is also disclosed, and any element(s) can be removed from the group. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, in certain contexts, “an element” means one element and/or in certain contexts more than one element. By way of another example, in certain contexts “a filler” means one filler and/or in certain contexts more than one filler (e.g., a mixture of two or more fillers).

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

At various places in the present specification, variables or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “XRPD” refers to X-ray powder diffraction. An XRPD pattern is an x-y graph with 20 (diffraction angle) plotted on the x-axis and intensity plotted on the y-axis. These are the diffraction peaks which may be used to characterize a crystalline material. The diffraction peaks are usually represented and referred to by their position on the x-axis rather than the intensity of the diffraction peaks on the y-axis because diffraction peak intensity can be particularly sensitive to sample orientation (see Pharmaceutical Analysis, Lee & Web, pp. 255-257 (2003)). Thus, intensity is not typically used by those of skill in the art to characterize a crystalline material. As with any data measurement, there may be variability in XRPD data. In addition to the variability in diffraction peak intensity, there may also be variability in the position of the diffraction peaks on the x-axis. This variability can, however, typically be accounted for when reporting the positions of diffraction peaks for purposes of characterization. Such variability in the position of diffraction peaks along the x-axis may be derived from several sources. One such source can be sample preparation. Samples of the same crystalline material prepared under different conditions may yield slightly different diffractograms. Factors such as particle size, moisture content, solvent content, temperature, and orientation may all affect how a sample diffracts X-rays. Another source of variability comes from instrument parameters. Different X-ray powder diffractometers operate using different parameters and may lead to slightly different diffraction patterns from the same crystalline material. Likewise, different software packages process XRPD data differently and this may also lead to variability. These and other sources of variability are known to those of ordinary skill in the art. Due to such sources of variability, the values of each X-ray diffraction peak may be preceded with the term “about” or proceeded with an appropriate range defining the experimental variability (e.g., ±0.1°, ±0.2°, ±0.3°, ±0.4°, ±0.5°, etc.).

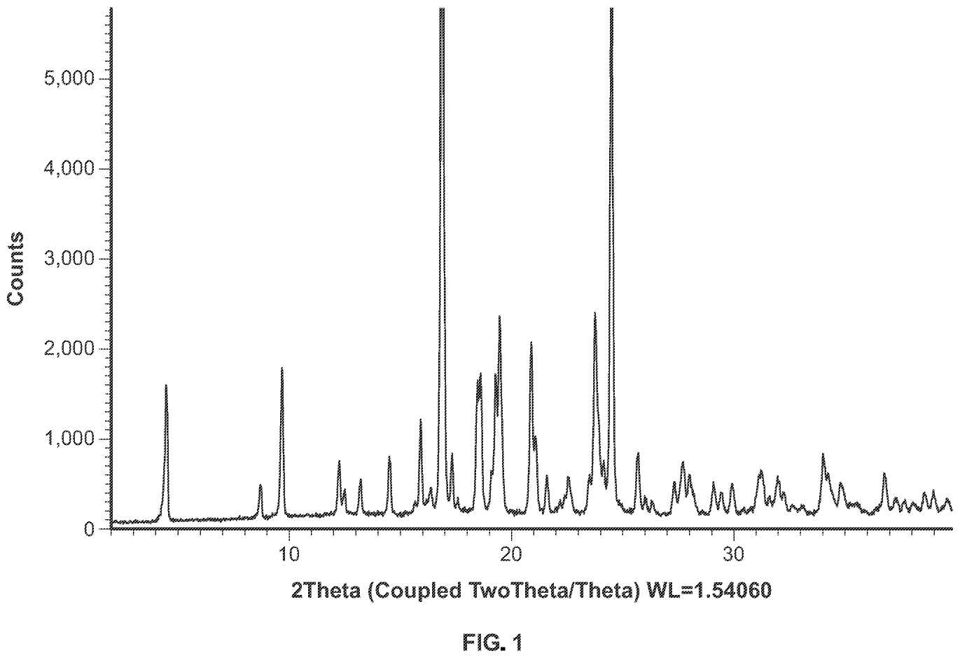

Crystalline forms, such as crystalline forms of a compound of Formula (I), are readily analyzed by XRPD. The data from x-ray powder diffraction may be used in multiple ways to characterize crystalline forms. For example, the entire x-ray powder diffraction pattern output from a diffractometer may be used to characterize a crystalline form (e.g., of a compound of Formula (I). A smaller subset of such data, however, may also be suitable and used for characterizing such crystalline forms. Indeed, often even a single x-ray powder diffraction peak may be used to characterize such a crystalline form. With respect to crystalline forms of a compound of Formula (I), any one or more of the peaks in the x-ray powder diffraction pattern of FIG. 1 may be used to characterize the crystalline form of a compound of Formula (I) disclosed herein.

The term “characteristic peaks” when referring to the peaks in an XRPD pattern of a crystalline form of a given chemical entity (e.g., a crystalline form of a compound of formula (I)) refers to a collection of specific diffraction peaks whose values span a range of 2θ values (e.g.,) 0°-40° that are, as a whole, unique to that specific crystalline form.

As used herein, “crystalline” refers to a solid phase of a given chemical entity having well-defined 3-dimensional structural order. The atoms, ions, and/or molecules are arranged in a regular, periodic manner within a repeating 3-dimensional lattice. In various embodiments, a crystalline material may comprise one or more discreet crystalline forms.

As used herein, the terms “crystalline form”, “crystalline solid form,” “crystal form,” “solid form,” and related terms herein refer to crystalline modifications comprising a given substance (e.g., the compound of formula (I)), including single-component crystal forms and multiple-component crystal forms, and including, but not limited to, polymorphs, solvates, hydrates, and salts.

The term “substantially crystalline” refers to solid forms that may be at least a particular weight percent crystalline. Particular weight percentages may include 70%, 75%, 80%, 85%, 87%, 88%, 89%, 90%, 91%, 92%, 93%, 94%, 95%, 96%, 97%, 98%, 99%, 99.5%, 99.9%, or any percentage between 70% and 100%. In certain embodiments, the particular weight percent of crystallinity is at least 90%. In certain other embodiments, the particular weight percent of crystallinity is at least 95%. In some embodiments, the compound of formula (I) can be a substantially crystalline sample of any of the crystalline solid forms described herein (e.g., a crystalline form with the XRPD pattern shown in FIG. 1).

The term “substantially pure” relates to the composition of a specific crystalline solid form (e.g., a crystalline form of the compound of formula (I)) that may be at least a particular weight percent free of impurities and/or other solid forms. Particular weight percentages may include 70%, 75%, 80%, 85%, 90%, 95%, 99%, or any percentage between 70% and 100%. In certain embodiments, the compound of formula (I) can be a substantially pure sample of any of the crystalline solid forms described herein, (e.g., a crystalline form with the XRPD pattern shown in FIG. 1 (Form A)). In certain embodiments, the compound of formula (I) can be a substantially pure crystalline form with the XRPD pattern shown in FIG. 1 (Form A).

As used herein, the term “anhydrous” or “anhydrate” when referring to a crystalline form (e.g., a crystalline form of the compound of formula (I)) means that no water molecules form a portion of the unit cell of the crystalline form. An anhydrous crystalline form may nonetheless contain water molecules that do not form part of the unit cell of the anhydrous crystalline form (e.g., as residual solvent molecule left behind from the production of the crystalline form). In a preferred embodiment, water can make up about 0.5% by weight of the total composition of a sample of an anhydrous form. In a more preferred embodiment, water can make up about 0.2% by weight of the total composition of a sample of an anhydrous form. In some embodiments, a sample of an anhydrous crystalline form of the compound of formula (I) contains no water molecules, e.g., no detectable amount of water.

As used herein, the term “desolvated” or “unsolvated” when referring to a crystalline form (e.g., a crystalline form of the compound of formula (I)) means that no solvent molecules form a portion of the unit cell of the crystalline form. An unsolvated crystalline form may nonetheless contain solvent molecules that do not form part of the unit cell of the unsolvated crystalline form (e.g., as residual solvent molecule left behind from the production of the crystalline form). In a preferred embodiment, the solvent can make up 0.5% by weight of the total composition of a sample of an unsolvated form. In a more preferred embodiment, solvent can make up 0.2% by weight of the total composition of a sample of an unsolvated form. In some embodiments, a sample of an unsolvated crystalline form of the compound of formula (I) contains no solvent molecules, e.g., no detectable amount of solvent.

As used herein, the terms “polymorph,” “polymorphic form,” “polymorphs,” “polymorphic forms” and related terms herein refer to two or more crystal forms that consist essentially of the same molecule, molecules, or ions (e.g., the compound of formula (I)). Different polymorphs may exhibit different physicochemical properties including, but not limited to, melting temperatures, solubilities, dissolution rates, and physical stabilities as a result of differences in the arrangement or conformation of the molecules or ions in the crystal lattice.

The term “solvate” when referring to a crystalline form of the compound of formula (I) means that solvent molecules (e.g., organic solvents and water), form a portion of the unit cell of the crystalline form. Solvates that contain water as the solvent are also referred to herein as “hydrates.”

As used herein, “dissolution profile” refers to dissolution testing of a drug substance or drug product at multiple time points. Dissolution profiles for drug substances (e.g., the compound of formula (I)) or drug products (e.g., the pharmaceutical compositions described herein) may be performed for characterization and quality control to ensure the drug is released at a defined range of rates in a well-defined dissolution aqueous media that is at least sink conditions for that drug, or in biorelevant media such as simulated gastric or intestinal fluids representing either the fasted or fed states. In certain cases, but not others, dissolution testing may be predictive of or give insight into in vivo bioavailability of the drug substance. Dissolution testing may be performed using USP testing protocols and dissolution apparatus.

As used herein, “granulation” refers to a process of forming granules from a powdered or particulate material. As used herein, “Dry granulation” refers to a process in which granules are formed without the presence of a liquid solution and may be useful in the preparation of granules of materials sensitive to heat, moisture, or solvents. For example, roller compaction is a dry granulation process. As used herein, “Wet granulation” refers to the formation of granules wherein the particles are bound together using a binder or a liquid solution. Examples of wet granulation are high shear granulation and fluid bed granulation.

As used herein, “pharmaceutical composition” or “pharmaceutical formulation” refer to the combination of a therapeutically active agent with a pharmaceutically acceptable excipient, inert or active, making the composition especially suitable for diagnostic or therapeutic use in vivo or ex vivo.

“Pharmaceutically acceptable” refers to compounds, molecular entities, compositions, materials and/or dosage forms that do not produce an adverse, allergic or other untoward reaction when administered to an animal, or human, as appropriate; or means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present disclosure (e.g., the compound of formula (I)), which salt is compatible with pharmaceutical administration.

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW4+, wherein W is C1-4 alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na+, K+, Ca2+, NH4+, and NW4+(where W can be a C1-4 alkyl group), and the like.

For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present disclosure without causing a significant adverse toxicological effect on the patient. Non-limiting examples of pharmaceutically acceptable excipients include binders, diluents, carriers, adjuvants, fillers (e.g., brittle diluents or fillers and ductile diluents or fillers), disintegrants, lubricants, coatings, sweeteners, flavors, gelatins, carbohydrates such as lactose, amylose or starch, fatty acid esters, hydroxypropylmethylcellulose, polyvinyl pyrrolidine, and colors, and the like. For examples of excipients, see Gennaro, Remington's Pharmaceutical Sciences, 18th Ed., Mack Publ. Co., Easton, PA (1990) or Shesky, Hancock, Moss and Goldfarb, Handbook of Pharmaceutical Excipients, 9th Ed. Pharmaceutical Press, London, UK (2020).

Examples of diluents or fillers include, but are not limited to, a sugar (e.g., mannitol, lactose, sorbitol, lactitol, erythritol, sucrose, fructose, glucose, agarose, maltose, isomalt, polydextrose, and combinations thereof), an inorganic material (e.g., dibasic calcium phosphate, hydroxyapatite, sodium carbonate, sodium bicarbonate, calcium carbonate, calcium sulfate, magnesium carbonate, magnesium oxide, bentonite, kaolin), calcium lactate, a starch (e.g., a pregelatinized starch), a microcrystalline cellulose, a silicified microcrystalline cellulose, a polysaccharide, a cellulose (e.g., a hydroxypropylcellulose, a hypromellose, a carboxymethylcellulose, a methylcellulose, a hydroxypropylmethylcellulose, a hydroxyethylcellulose), a dextrin, a maltodextrin, an alginate, a collagen, a polyvinylpyrrolidone, a polyvinylacrylate, polyethylene oxide, and polyethylene glycol. Sugar is defined herein to include sugar alcohols.

Examples of disintegrants include, but are not limited to, alginic acid, an alginate, primogel, a cellulose (e.g., hydroxypropylcellulose), polacrilin potassium, sodium starch glycolate, sodium croscarmellose, a polyplasdone (e.g., a crospovidone), and a starch (e.g., corn starch, pregelatinized starch, hydroxypropyl starch, and carboxymethyl starch).

Examples of binders include, but are not limited to, a hydroxypropylcellulose, hydroxyethylcellulose, a hydroxypropylmethycellulose (e.g., a low viscosity hydroxypropylmethycellulose), a sugar, a polyvinylpyrrolidone, a polyvinyl alcohol, a polyvinyl acetate, a polydextrose, a chitosan, a carrageenan, carbophil, a microcrystalline cellulose, gum tragacanth, guar gum, gellan gum, gelatin, and a starch (e.g., corn starch).

Examples of wetting agents include, but are not limited to, a poloxamer (e.g., poloxamer 407), sodium dodecyl sulfate, sodium lauryl sulfate (SLS), sodium stearyl fumarate (SSF), a polydimethylsiloxane, a polysorbate (e.g., polyoxyethylene 20 sorbitan mono-oleate (Tween® 20)), sorbitan monooleate, sorbitan trioleate, sorbitan laurate, sorbitan stearate, sorbitan monopalmitate, lecithin, sodium taurocholate, ursodeoxycholate, polyethoxylated castor oil, cetyl trimethylammonium bromide, nonoxynol, \acute{α}-tocopherol polyethylene glycol 1000 succinate, and docusate sodium.

Examples of lubricants and glidants include, but are not limited to, a wax, a glyceride, a light mineral oil, a polyethylene glycol, sodium stearyl fumarate, magnesium stearate, stearic acid, hydrogenated oil (e.g., hydrogenated vegetable oil), an alkyl sulfate, sodium benzoate, sodium acetate, glyceryl behenate, palmitic acid, and coconut oil.

Examples of glidants include, but are not limited to, colloidal silicon dioxide, colloidal silicon dioxide, talc, kaolin, bentonite, and activated carbon/charcoal.

Examples of colorants include, but are not limited to, titanium dioxide, aluminum lakes, iron oxides and carbon black.

Examples of coatings include but are not limited to, a film forming polymer (e.g., a hypromellose, a methyl cellulose, an ethylcellulose, cellulose acetate, a hydroxypropylmethyl cellulose, a hydroxypropyl cellulose, hydroxypropylmethyl cellulose acetate succinate, cellulose acetate phthalate, a polyvinylpyrrolidone, polyvinyl alcohol, a Eudragit/acrylate) and a plasticizer (e.g., triacetin, polyethylene glycol, propylene glycol).

Pharmaceutical compositions for oral administration (e.g., pharmaceutical compositions of the compound of formula (I) described herein) can take the form of bulk liquid solutions or suspensions or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pills, tablets, capsules or the like in the case of solid compositions.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “solid dosage form” means a pharmaceutical dose(s) in solid form, e.g., tablets, capsules, granules, powders, minitabs, sachets, stickpacks, reconstitutable powders, dry powder inhalers, lozenges, and chewables.

As used herein, “administering” means oral administration, administration as a pulmonary, suppository, intramuscular administration, intrathecal administration, intranasal administration or subcutaneous administration, or the implantation of a slow-release device, e.g., a mini-osmotic pump, to a subject. Administration is by any route, including transmucosal (e.g., buccal, sublingual, palatal, gingival, nasal, vaginal, rectal, or). Parenteral administration includes, e.g., intramuscular and subcutaneous. Other modes of delivery include, but are not limited to, the use of liposomal formulations, etc. By “co-administer” it is meant that a composition described herein is administered at the same time, just prior to, or just after the administration of one or more additional therapies (e.g., anti-cancer agent, chemotherapeutic, or treatment for a neurodegenerative disease). The compound of formula (I) can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). In one embodiment, the compounds provided herein are contemplated to be used in methods of therapeutic treatment wherein the action occurs while a subject is suffering from the specified disease, disorder or condition and results in a reduction in the severity of the disease, disorder or condition, or retardation or slowing of the progression of the disease, disorder or condition. In an alternate embodiment, the compounds provided herein are contemplated to be used in methods of prophylactic treatment wherein the action occurs before a subject begins to suffer from the specified disease, disorder or condition and results in preventing a disease, disorder or condition, or one or more symptoms associated with the disease, disorder or condition, or preventing the recurrence of the disease, disorder or condition.

In general, the “effective amount” of a compound refers to an amount sufficient to elicit the desired biological response e.g., to treat a disease or disorder described herein. As will be appreciated by those of ordinary skill in this art, the effective amount of a compound of the disclosure may vary depending on such factors as the desired biological endpoint, the pharmacokinetics of the compound, the disease being treated, the mode of administration, and the age, health, and condition of the subject. An effective amount encompasses therapeutic and prophylactic treatment (i.e., encompasses a “therapeutically effective amount” and a “prophylactically effective amount”).

As used herein, and unless otherwise specified, a “therapeutically effective amount” of a compound is an amount sufficient to provide a therapeutic benefit in the therapeutic treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the therapeutic treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### Compound

In some embodiments, provided herein is N-(6-amino-5-ethylpyridin-3-yl)-2-((2R,5S)-5-methyl-2-(2-(1-methylpiperidin-4-yl)benzo[d]thiazol-5-yl)piperidin-1-yl)-2-oxoacetamide, a PRMT5 inhibitor (e.g., an MTA-uncompetitive PRMT5 inhibitor) compound of formula (I).

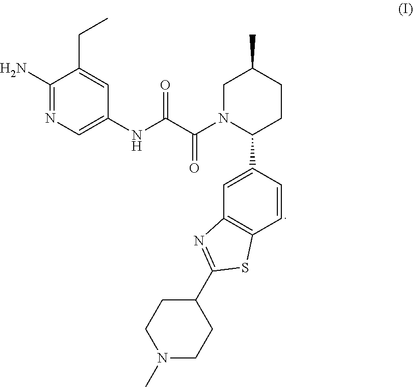 [SMILES: CCc1cc(NC(=O)C(=O)N2C[C@@H](C)CC[C@@H]2c2ccc3sc(C4CCN(C)CC4)nc3c2)cnc1N]

In certain embodiments, the compound of formula (I) is a crystalline form of the compound of formula (I). The compound of formula (I) can also be referred to as “Compound I.”

In certain embodiments, the crystalline form of the compound of formula (I) is crystalline Form A. In certain embodiments, Form A has an XRPD pattern with one or more (e.g., one, two, three or four) characteristic peaks between and including the following values of 20 in degrees: 4.2 to 4.6 (e.g., 4.4±0.2), 9.4 to 9.8 (e.g., 9.6±0.2), 16.6 to 17.0 (e.g., 16.8±0.2) and 24.3 to 24.7 (e.g., 24.5±0.2). In certain embodiments, Form A has an XRPD pattern comprising characteristic peaks between and including the following values of 20 in degrees: 4.2 to 4.6 (e.g., 4.4±0.2), 9.4 to 9.8 (e.g., 9.6±0.2), 16.6 to 17.0 (e.g., 16.8±0.2) and 24.3 to 24.7 (e.g., 24.5±0.2).

In certain embodiments, Form A has an XRPD pattern with one or more (e.g., one, two, three, four, five, six, seven or eight) characteristic peaks between and including the following values of 20 in degrees: 4.2 to 4.6 (e.g., 4.4±0.2), 9.4 to 9.8 (e.g., 9.6±0.2), 16.6 to 17.0 (e.g., 16.8±0.2), 18.4 to 18.8 (e.g., 18.6±0.2), 19.2 to 19.6 (e.g., 19.4±0.2), 20.7 to 21.1 (e.g., 20.9±0.2), 23.5 to 23.9 (e.g., 23.7±0.2) and 24.3 to 24.7 (e.g., 24.5±0.2). In certain embodiments, Form A has an XRPD pattern comprising characteristic peaks between and including the following values of 20 in degrees: 4.2 to 4.6 (e.g., 4.4±0.2), 9.4 to 9.8 (e.g., 9.6±0.2), 16.6 to 17.0 (e.g., 16.8±0.2) and 24.3 to 24.7 (e.g., 24.5±0.2) and at least one additional characteristic peak selected from peaks between and including the following values of 20 in degrees: 18.4 to 18.8 (e.g., 18.6±0.2), 19.2 to 19.6 (e.g., 19.4±0.2), 20.7 to 21.1 (e.g., 20.9±0.2), 23.5 to 23.9 (e.g., 23.7±0.2).

In certain embodiments, Form A has an XRPD pattern comprising characteristic peaks between and including the following values of 20 in degrees: 4.2 to 4.6 (e.g., 4.4±0.2), 9.4 to 9.8 (e.g., 9.6±0.2), 16.6 to 17.0 (e.g., 16.8±0.2), 18.4 to 18.8 (e.g., 18.6±0.2), 19.2 to 19.6 (e.g., 19.4±0.2), 20.7 to 21.1 (e.g., 20.9±0.2), 23.5 to 23.9 (e.g., 23.7±0.2) and 24.3 to 24.7 (e.g., 24.5±0.2).

In certain embodiments, Form A has an XRPD pattern with one or more (e.g., one, two, three, four, five, six, seven, eight, nine or ten) characteristic peaks between and including the following values of 20 in degrees: 4.2 to 4.6 (e.g., 4.4±0.2), 9.4 to 9.8 (e.g., 9.6±0.2), 15.7 to 16.1 (e.g., 15.9±0.2), 16.6 to 17.0 (e.g., 16.8±0.2), 18.4 to 18.8 (e.g., 18.6±0.2), 19.1 to 19.5 (e.g., 19.3±0.2), 19.2 to 19.6 (e.g., 19.4±0.2), 20.7 to 21.1 (e.g., 20.9±0.2), 20.8 to 21.2 (e.g., 21.0±0.2), 23.5 to 23.9 (e.g., 23.7±0.2), 24.3 to 24.5 (e.g., 24.5±0.2), 25.5 to 25.9 (e.g., 25.7±0.2) and 33.8 to 34.2 (e.g., 34.0±0.2).

In certain embodiments, the X-ray powder diffraction pattern for Form A may comprise one or more (e.g., one, two, three or four) characteristic peaks, in terms of 20, selected from the peaks at degrees: 4.4±0.2, 9.6±0.2, 16.8±0.2 and 24.5±0.2. In certain embodiments, the X-ray powder diffraction pattern for Form A comprises at least one characteristic peak, in terms of 20, selected from the peaks at degrees: 4.4±0.2, 9.6±0.2, 16.8±0.2 and 24.5±0.2. In certain embodiments, the X-ray powder diffraction pattern for Form A comprises at least two characteristic peaks, in terms of 20, selected from the peaks at degrees: 4.4±0.2, 9.6±0.2, 16.8±0.2 and 24.5±0.2. In certain embodiments, the X-ray powder diffraction pattern for Form A comprises at least three characteristic peaks, in terms of 20, selected from the peaks at degrees: 4.4±0.2, 9.6±0.2, 16.8±0.2 and 24.5±0.2.

In certain embodiments, Form A has an XRPD pattern comprising characteristic peaks at the following values of 20 in degrees: 4.4±0.2, 9.6±0.2, 16.8±0.2 and 24.5±0.2.

In certain embodiments, the X-ray powder diffraction pattern for Form A may comprise one or more (e.g., one, two, three, four, five, six, seven or eight) characteristic peaks, in terms of 20, selected from the peaks at 4.4±0.2, 9.6±0.2, 16.8±0.2, 18.6±0.2, 19.4±0.2, 20.9±0.2, 23.7±0.2 and 24.5±0.2.

In certain embodiments, the X-ray powder diffraction pattern for Form A comprises at least one characteristic peak, in terms of 20, selected from the peaks at 4.4±0.2, 9.6±0.2, 16.8±0.2, 18.6±0.2, 19.4±0.2, 20.9±0.2, 23.7±0.2 and 24.5±0.2. In certain embodiments, the X-ray powder diffraction pattern for Form A comprises at least two characteristic peaks, in terms of 20, selected from the peaks at 4.4±0.2, 9.6±0.2, 16.8±0.2, 18.6±0.2, 19.4±0.2, 20.9±0.2, 23.7±0.2 and 24.5±0.2. In certain embodiments, the X-ray powder diffraction pattern for Form A comprises at least three characteristic peaks, in terms of 20, selected from the peaks at 4.4±0.2, 9.6±0.2, 16.8±0.2, 18.6±0.2, 19.4±0.2, 20.9±0.2, 23.7±0.2 and 24.5±0.2. In certain embodiments, the X-ray powder diffraction pattern for Form A comprises at least four characteristic peaks, in terms of 20, selected from the peaks at 4.4±0.2, 9.6±0.2, 16.8±0.2, 18.6±0.2, 19.4±0.2, 20.9±0.2, 23.7±0.2 and 24.5±0.2. In certain embodiments, the X-ray powder diffraction pattern for Form A comprises at least five characteristic peaks, in terms of 20, selected from the peaks at 4.4±0.2, 9.6±0.2, 16.8±0.2, 18.6±0.2, 19.4±0.2, 20.9±0.2, 23.7±0.2 and 24.5±0.2. In certain embodiments, the X-ray powder diffraction pattern for Form A comprises at least six characteristic peaks, in terms of 20, selected from the peaks at 4.4±0.2, 9.6±0.2, 16.8±0.2, 18.6±0.2, 19.4±0.2, 20.9±0.2, 23.7±0.2 and 24.5±0.2. In certain embodiments, the X-ray powder diffraction pattern for Form A comprises at least seven characteristic peaks, in terms of 20, selected from the peaks at 4.4±0.2, 9.6±0.2, 16.8±0.2, 18.6±0.2, 19.4±0.2, 20.9±0.2, 23.7±0.2 and 24.5±0.2.

In certain embodiments, Form A has an XRPD pattern comprising characteristic peaks at the following values of 20 in degrees: 4.4±0.2, 9.6±0.2, 16.8±0.2, 19.3±0.2, 19.4±0.2, 20.9±0.2, 23.7±0.2, 24.5±0.2 and at least one additional characteristic peak at a value of 20 in degrees selected from: 19.3±0.2, 19.4±0.2, 20.9±0.2 and 23.7±0.2. In certain embodiments, Form A has an XRPD pattern comprising characteristic peaks at the following values of 20 in degrees: 4.4±0.2, 9.6±0.2, 16.8±0.2, 19.3±0.2, 19.4±0.2, 20.9±0.2, 23.7±0.2, 24.5±0.2 and at least two additional characteristic peaks at a value of 20 in degrees selected from: 19.3±0.2, 19.4±0.2, 20.9±0.2 and 23.7±0.2. In certain embodiments, Form A has an XRPD pattern comprising characteristic peaks at the following values of 20 in degrees: 4.4±0.2, 9.6±0.2, 16.8±0.2, 19.3±0.2, 19.4±0.2, 20.9±0.2, 23.7±0.2, 24.5±0.2 and at least three additional characteristic peak at a value of 20 in degrees selected from: 19.3±0.2, 19.4±0.2, 20.9±0.2 and 23.7±0.2.

In certain embodiments, Form A has an XRPD pattern comprising characteristic peaks at the following values of 20 in degrees: 4.4±0.2, 9.6±0.2, 16.8±0.2, 18.6±0.2, 19.4±0.2, 20.9±0.2, 23.7±0.2, 24.5±0.2.

In certain embodiments, the X-ray powder diffraction pattern for Form A may comprise one or more (e.g., one, two, three, four, five, six, seven, eight, nine, or ten) characteristic peaks, in terms of 20, selected from the peaks at 4.4±0.2, 9.6±0.2, 15.9±0.2, 16.8±0.2, 18.6±0.2, 19.3±0.2, 19.4±0.2, 20.9±0.2, 21.0±0.2, 23.7±0.2, 24.5±0.2, 25.7±0.2 and 34.0±0.2.

In certain embodiments, the X-ray powder diffraction pattern for Form A comprises at least one characteristic peak, in terms of 20, selected from the peaks at 4.4±0.2, 9.6±0.2, 15.9±0.2, 16.8±0.2, 18.6±0.2, 19.3±0.2, 19.4±0.2, 20.9±0.2, 21.0±0.2, 23.7±0.2, 24.5±0.2, 25.7±0.2 and 34.0±0.2. In certain embodiments, the X-ray powder diffraction pattern for Form A comprises at least two characteristic peaks, in terms of 20, selected from the peaks at 4.4±0.2, 9.6±0.2, 15.9±0.2, 16.8±0.2, 18.6, 19.3±0.2, 19.4±0.2, 20.9±0.2, 21.0±0.2, 23.7±0.2, 24.5±0.2, 25.7±0.2 and 34.0±0.2. In certain embodiments, the X-ray powder diffraction pattern for Form A comprises at least three characteristic peaks, in terms of 20, selected from the peaks at 4.4±0.2, 9.6±0.2, 15.9±0.2, 16.8±0.2, 18.6, 19.3±0.2, 19.4±0.2, 20.9±0.2, 21.0±0.2, 23.7±0.2, 24.5±0.2, 25.7±0.2 and 34.0±0.2. In certain embodiments, the X-ray powder diffraction pattern for Form A comprises at least four characteristic peaks, in terms of 20, selected from the peaks at 4.4±0.2, 9.6±0.2, 15.9±0.2, 16.8±0.2, 18.6, 19.3±0.2, 19.4±0.2, 20.9±0.2, 21.0±0.2, 23.7±0.2, 24.5±0.2, 25.7±0.2 and 34.0±0.2. In certain embodiments, the X-ray powder diffraction pattern for Form A comprises at least five characteristic peaks, in terms of 20, selected from the peaks at 4.4±0.2, 9.6±0.2, 15.9±0.2, 16.8±0.2, 18.6, 19.3±0.2, 19.4±0.2, 20.9±0.2, 21.0±0.2, 23.7±0.2, 24.5±0.2, 25.7±0.2 and 34.0±0.2. In certain embodiments, the X-ray powder diffraction pattern for Form A comprises at least six characteristic peaks, in terms of 20, selected from the peaks at 4.4±0.2, 9.6±0.2, 15.9±0.2, 16.8±0.2, 18.6, 19.3±0.2, 19.4±0.2, 20.9±0.2, 21.0±0.2, 23.7±0.2, 24.5±0.2, 25.7±0.2 and 34.0±0.2. In certain embodiments, the X-ray powder diffraction pattern for Form A comprises at least seven characteristic peaks, in terms of 20, selected from the peaks at 4.4±0.2, 9.6±0.2, 15.9±0.2, 16.8±0.2, 18.6, 19.3±0.2, 19.4±0.2, 20.9±0.2, 21.0±0.2, 23.7±0.2, 24.5±0.2, 25.7±0.2 and 34.0±0.2. In certain embodiments, the X-ray powder diffraction pattern for Form A comprises at least eight characteristic peaks, in terms of 20, selected from the peaks at 4.4±0.2, 9.6±0.2, 15.9±0.2, 16.8±0.2, 18.6, 19.3±0.2, 19.4±0.2, 20.9±0.2, 21.0±0.2, 23.7±0.2, 24.5±0.2, 25.7±0.2 and 34.0±0.2. In certain embodiments, the X-ray powder diffraction pattern for Form A comprises at least nine characteristic peaks, in terms of 20, selected from the peaks at 4.4±0.2, 9.6±0.2, 15.9±0.2, 16.8±0.2, 18.6, 19.3±0.2, 19.4±0.2, 20.9±0.2, 21.0±0.2, 23.7±0.2, 24.5±0.2, 25.7±0.2 and 34.0±0.2. In certain embodiments, the X-ray powder diffraction pattern for Form A comprises at least ten characteristic peaks, in terms of 20, selected from the peaks at 4.4±0.2, 9.6±0.2, 15.9±0.2, 16.8±0.2, 18.6, 19.3±0.2, 19.4±0.2, 20.9±0.2, 21.0±0.2, 23.7±0.2, 24.5±0.2, 25.7±0.2 and 34.0±0.2.

In certain embodiments, Form A has an XRPD pattern comprising characteristic peaks at the following values of 20 in degrees: 4.4±0.2, 9.6±0.2, 15.9±0.2, 16.8±0.2, 18.6, 19.3±0.2, 19.4±0.2, 20.9±0.2, 21.0±0.2, 23.7±0.2, 24.5±0.2 and 25.7±0.2, 34.0±0.2.

In some embodiments, Form A has an XRPD pattern (obtained using CuKa radiation) substantially corresponding to the XRPD diffraction pattern shown in FIG. 1.

In some embodiments, Form A has an XRPD pattern (obtained using CuKa radiation) with characteristic peaks comprising one, two, three, four, five, six, seven, eight, nine, or ten characteristic peaks, in terms of 2θ values in degrees shown in Table 1 (±0.2 degrees).

TABLE 1
XRPD peaks for Form A of a compound of Formula (I)

| | | Relative |
|---|---|---|
| Peak | 2θ Angle | intensity |
| 1 | 4.40 | 15.5% |
| 2 | 8.65 | 3.9% |
| 3 | 9.62 | 18.6% |
| 4 | 12.20 | 6.9% |
| 5 | 12.44 | 3.0% |
| 6 | 13.17 | 4.4% |
| 7 | 14.47 | 7.2% |
| 8 | 15.87 | 12.5% |
| 9 | 16.35 | 2.9% |
| 10 | 16.84 | 100% |
| 11 | 17.29 | 7.4% |
| 12 | 17.55 | 1.9% |
| 13 | 18.57 | 17.1% |
| 14 | 19.26 | 18.0% |
| 15 | 19.44 | 24.7% |
| 16 | 20.86 | 22.3% |
| 17 | 21.04 | 10.3% |
| 18 | 21.56 | 5.1% |
| 19 | 22.53 | 4.6% |
| 20 | 23.49 | 4.6% |
| 21 | 23.74 | 26.9% |
| 22 | 24.12 | 6.8% |
| 23 | 24.48 | 77.0% |
| 24 | 25.67 | 7.6% |
| 25 | 25.99 | 2.7% |
| 26 | 26.32 | 1.7% |
| 27 | 27.31 | 4.5% |
| 28 | 27.69 | 7.1% |
| 29 | 27.99 | 5.3% |
| 30 | 29.08 | 4.1% |
| 31 | 29.43 | 2.7% |
| 32 | 29.92 | 4.0% |
| 33 | 30.43 | 0.7% |
| 34 | 31.24 | 5.3% |
| 35 | 31.59 | 2.3% |
| 36 | 31.97 | 5.0% |
| 37 | 32.23 | 2.9% |
| 38 | 32.67 | 0.8% |
| 39 | 33.09 | 0.8% |
| 40 | 34.03 | 8.1% |
| 41 | 34.80 | 4.3% |
| 42 | 35.48 | 0.9% |
| 43 | 36.78 | 6.2% |
| 44 | 37.27 | 2.0% |
| 45 | 37.71 | 1.1% |
| 46 | 38.08 | 1.1% |
| 47 | 38.58 | 2.6% |
| 48 | 38.99 | 2.9% |
| 49 | 39.63 | 1.4% |

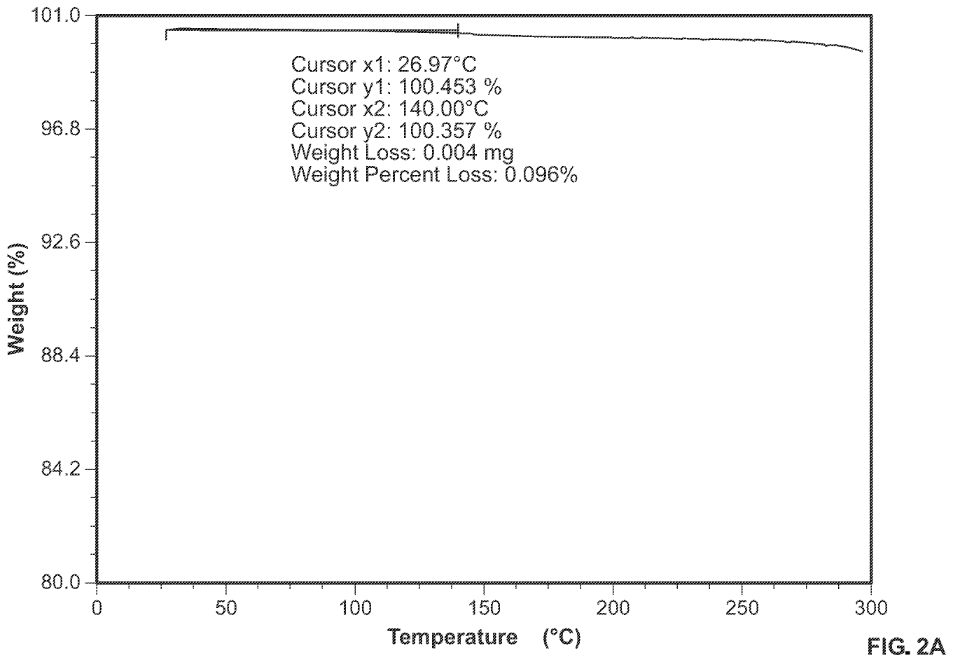

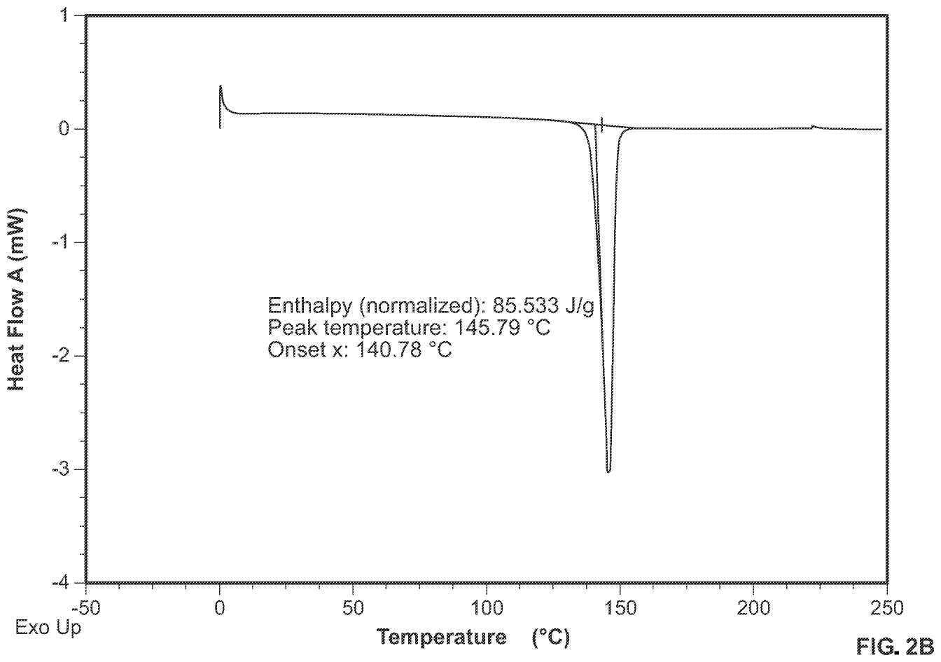

In certain embodiments, Form A is substantially characterized by the thermal gravimetric analysis (TGA) as shown in FIG. 2A.

In certain embodiments, Form A is substantially characterized by the differential scanning calorimetry profile (DSC) shown in FIG. 2B. In some embodiments, Form A can be characterized by the differential scanning calorimetry profile (DSC) shown in FIG. 2B, showing a melt peak at about 145.8° C.

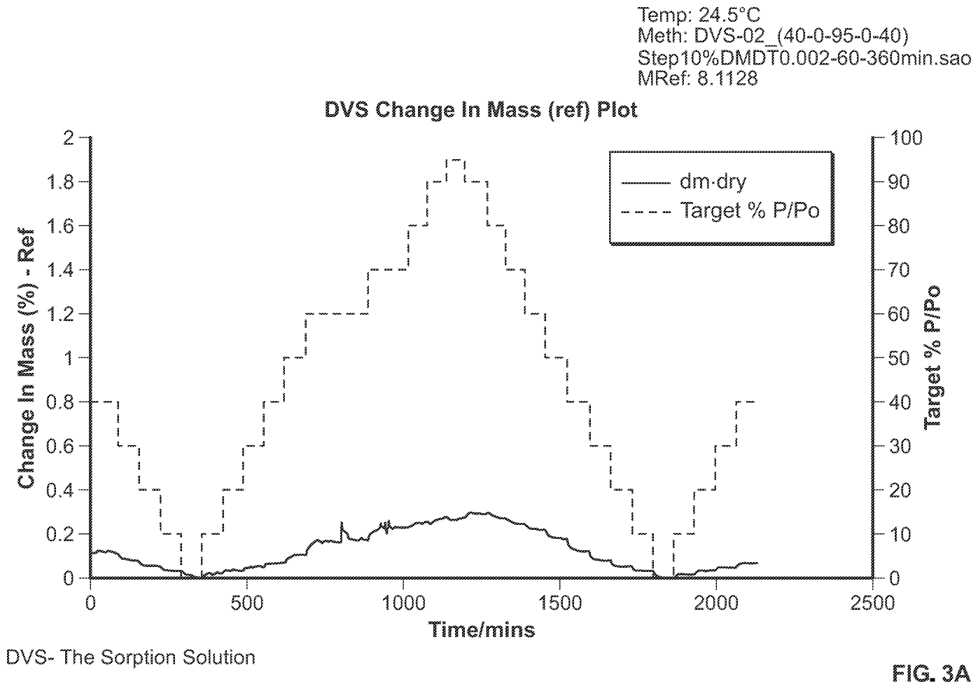

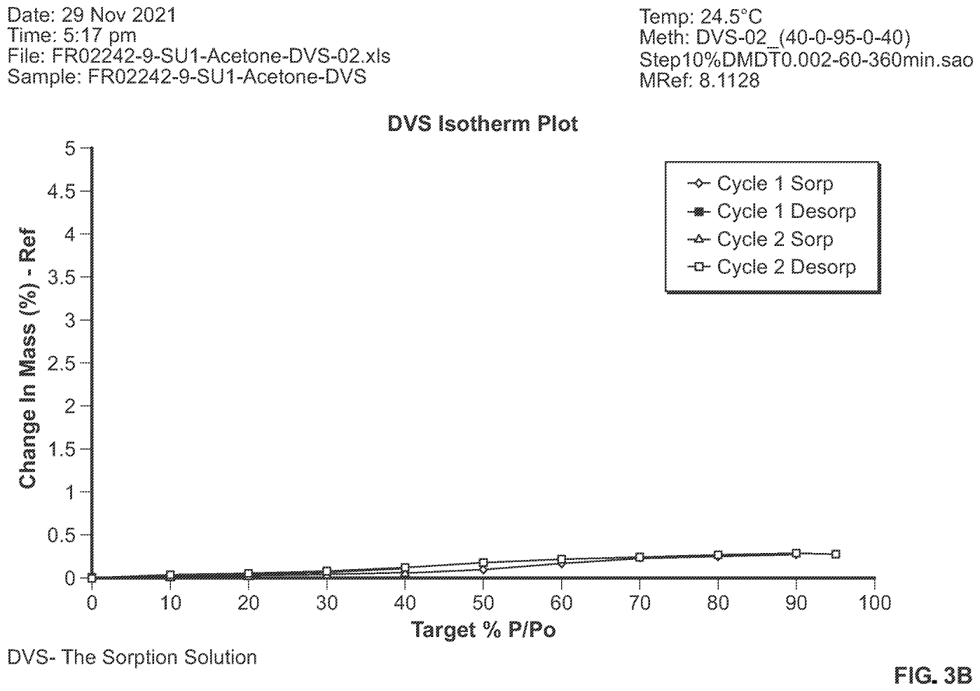

In certain embodiments, Form A is substantially characterized by the DVS profile as shown in FIG. 3A and FIG. 3B.

### Pharmaceutical Compositions

In some embodiments, provided are pharmaceutical compositions comprising a compound of formula (I)

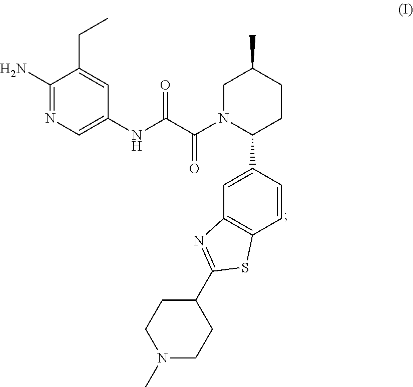 [SMILES: CCc1cc(NC(=O)C(=O)N2C[C@@H](C)CC[C@@H]2c2ccc3sc(C4CCN(C)CC4)nc3c2)cnc1N]

or a pharmaceutically acceptable salt thereof and at least one pharmaceutically acceptable excipient.

In some embodiments, provided are pharmaceutical compositions comprising a compound of formula (I)

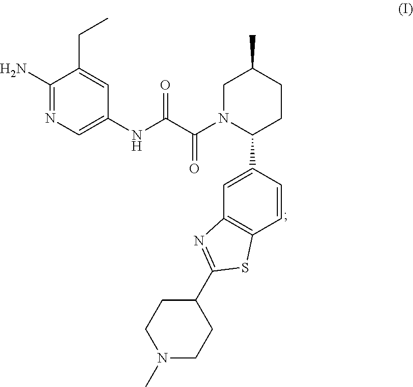 [SMILES: CCc1cc(NC(=O)C(=O)N2C[C@@H](C)CC[C@@H]2c2ccc3sc(C4CCN(C)CC4)nc3c2)cnc1N]

as the free base and at least one pharmaceutically acceptable excipient.

In some embodiments, provided are pharmaceutical compositions comprising as the pharmaceutically active ingredient a compound of formula (I)

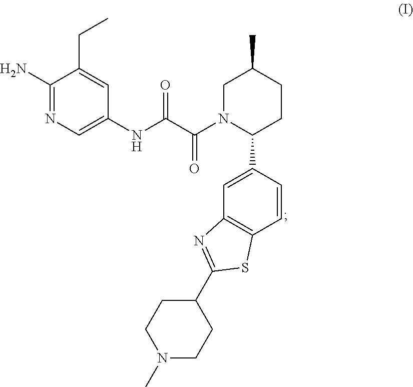 [SMILES: CCc1cc(NC(=O)C(=O)N2C[C@@H](C)CC[C@@H]2c2ccc3sc(C4CCN(C)CC4)nc3c2)cnc1N]

or a pharmaceutically acceptable salt thereof and at least one pharmaceutically acceptable excipient.

In some embodiments, the pharmaceutically active ingredient is the free base of the compound of formula (I).

In some embodiments, the composition comprises a crystalline form of the compound of Formula (I). In some embodiments, the composition comprises crystalline form A of the compound of Formula (I) as described herein and at least one pharmaceutically acceptable excipient.

The amounts and ranges described below as pertaining to “a compound of formula (I)” can refer to the compound of formula (I) generally, to a crystalline form of a compound of formula (I), or to the crystalline form A of the compound of formula (I) described herein. For compositions and dosage forms comprising a pharmaceutically acceptable salt of a compound of Formula (I), the amounts and ranges described below as pertaining to “a compound of formula (I)” refer to the equivalent dose of the free base of the compound contained in the composition or dosage form.

In some embodiments, the pharmaceutical composition comprises about 2% (w/w) to about 20% (w/w) of the compound of formula (I).

In some embodiments, the pharmaceutical composition comprises about 3% (w/w) to about 17% (w/w) of the compound of formula (I).

In some embodiments, the pharmaceutical composition comprises about 5% (w/w) to about 15% (w/w) of a compound of formula (I). In some embodiments, the pharmaceutical composition comprises about 8% (w/w) to about 12% (w/w) of a compound of formula (I).

In some embodiments, the pharmaceutical composition comprises about 10% (w/w) of a compound of formula (I).

In some embodiments, the pharmaceutical composition comprises a filler. In certain embodiments, the filler is selected from the group consisting of a sugar, an inorganic material, a microcrystalline cellulose, a starch, a polysaccharide, a cellulose, a polyvinylpyrrolidone, a polyvinyl acrylate and combinations thereof.

In certain embodiments, the filler is selected from the group consisting of a sugar, an inorganic material, and combinations thereof. In certain embodiments, the sugar is selected from the group consisting of mannitol, lactose, sucrose, fructose, glucose, maltose, and combinations thereof. In certain embodiments, the inorganic material is selected from the group consisting of dibasic calcium phosphate, hydroxyapatite, sodium carbonate, sodium bicarbonate, calcium carbonate, bentonite, kaolin, and combinations thereof.

In certain embodiments, the filler is selected from the group consisting of a microcrystalline cellulose, a starch, a polysaccharide, a cellulose, a polyvinylpyrrolidone, a polyvinyl acrylate, and combinations thereof. In certain embodiments, the cellulose is selected from the group consisting of a hydroxypropylcellulose, a hypromellose, a carboxymethylcellulose, a methylcellulose, a hydroxypropylmethylcellulose, and combinations thereof.

In some embodiments, the filler is microcrystalline cellulose (e.g., Avicel®). The microcrystalline cellulose filler can be of different grades. In some embodiments, the filler is microcrystalline cellulose PH 102, PH 200 or a mixture thereof. In some embodiments, the filler is microcrystalline cellulose PH 102 (e.g., Avicel® PH 102). In some embodiments, the filler is microcrystalline cellulose PH 200 (e.g., Avicel® PH 200). In some embodiments, the filler is a mixture of microcrystalline cellulose PH 102 (e.g., Avicel® PH 102) and microcrystalline cellulose PH 200 (e.g., Avicel® PH 200). In further embodiments, the filler contains equal amounts of the two grades of microcrystalline cellulose.

In certain embodiments, the pharmaceutical composition comprises about 50% (w/w) to about 90% (w/w) filler.

In certain embodiments, the pharmaceutical composition comprises about 75% (w/w) to about 86% (w/w) filler. In certain embodiments, the pharmaceutical composition comprises about 77% (w/w) to about 85% (w/w) filler. In certain embodiments, the pharmaceutical composition comprises about 80% (w/w) to about 85% (w/w) filler. In certain embodiments, the pharmaceutical composition comprises about 82% (w/w) to about 84% (w/w) filler.

In certain embodiments, the pharmaceutical composition comprises about 75% (w/w), about 76% (w/w), about 77% (w/w), about 78% (w/w), about 79% (w/w), about 80% (w/w), about 81% (w/w), about 82% (w/w), about 83% (w/w), about 83.5% (w/w), about 84% (w/w), about 85% (w/w), about 86% (w/w) filler. In certain embodiments, the pharmaceutical composition comprises about 80% (w/w), about 81% (w/w), about 82% (w/w), about 83% (w/w), about 83.5% (w/w), about 84% (w/w), about 85% (w/w), about 86% (w/w) filler.

In certain embodiments, the pharmaceutical composition comprises about 83.5% (w/w) filler.

In some embodiments, the pharmaceutical composition comprises a glidant. In certain embodiments, the glidant is selected from the group consisting of colloidal silicon dioxide, talc, kaolin, bentonite, or combinations thereof.

In further embodiments, the glidant is colloidal silicon dioxide. In some embodiments, the colloidal silicon dioxide is prepared through a process involving flame hydrolysis of silicon tetrachloride in an oxy-hydrogen flame and is referred to as “fumed silica” or “untreated fumed silica” (e.g., Aerosil® 200, CAB-O-SIL® M-5P).

In certain embodiments, the pharmaceutical composition comprises about 0.5% (w/w) to about 2.5% (w/w) glidant. In certain embodiments, the pharmaceutical composition comprises about 0.75% (w/w) to about 2.25% (w/w) glidant. In certain embodiments, the pharmaceutical composition comprises about 1% (w/w) to about 2% (w/w) glidant. In certain embodiments, the pharmaceutical composition comprises about 1.2% (w/w) to about 1.8% (w/w) glidant. In certain embodiments, the pharmaceutical composition comprises about 1.4% (w/w) to about 1.6% (w/w) glidant. In certain embodiments, the pharmaceutical composition comprises about 1.45% (w/w) to about 1.55% (w/w) glidant.

In certain embodiments, the pharmaceutical composition comprises about 1.4% (w/w), about 1.42% (w/w), about 1.44% (w/w), about 1.46% (w/w), about 1.48% (w/w), about 1.5% (w/w), about 1.52% (w/w), about 1.54% (w/w), about 1.56% (w/w), about 1.58% (w/w) or about 1.6% (w/w) glidant.

In certain embodiments, the pharmaceutical composition comprises about 1.5% (w/w) glidant.

In some embodiments, the pharmaceutical composition comprises a disintegrant. In certain embodiments, the disintegrant is selected from the group consisting of sodium starch glycolate, a crospovidone, croscarmellose sodium, and combinations thereof. In further embodiments, the disintegrant is croscarmellose sodium (e.g., Ac-Di-Sol®).

In certain embodiments, the pharmaceutical composition comprises about 2% (w/w) to about 6% (w/w) disintegrant. In certain embodiments, the pharmaceutical composition comprises about 3% (w/w) to about 5% (w/w) disintegrant. In certain embodiments, the pharmaceutical composition comprises about 3.6% (w/w) to about 4.4% (w/w) disintegrant. In certain embodiments, the pharmaceutical composition comprises about 3.8% (w/w) to about 4.2% (w/w) disintegrant.

In certain embodiments, the pharmaceutical composition comprises about 3.2% (w/w), about 3.28% (w/w), about 3.36% (w/w), about 3.44% (w/w), about 3.52% (w/w), about 3.6% (w/w), about 3.68% (w/w), about 3.76% (w/w), about 3.84% (w/w), about 3.92% (w/w), about 4% (w/w), about 4.08% (w/w), about 4.16% (w/w), about 4.24% (w/w), about 4.32% (w/w), about 4.4% (w/w), about 4.48% (w/w), about 4.56% (w/w), about 4.64% (w/w), about 4.72% (w/w), or about 4.8% (w/w) disintegrant. In certain embodiments, the pharmaceutical composition comprises about 3.84% (w/w), about 3.92% (w/w), about 4% (w/w), about 4.08% (w/w), about 4.16% (w/w), about 4.24% (w/w), about 4.32% (w/w), about 4.4% (w/w), about 4.48% (w/w), about 4.56% (w/w), about 4.64% (w/w), about 4.72% (w/w), or about 4.8% (w/w) disintegrant. In certain embodiments, the pharmaceutical composition comprises about 3.92% (w/w), about 4.00% (w/w) or about 4.08% (w/w) disintegrant.

In certain embodiments, the pharmaceutical composition comprises about 4% (w/w) disintegrant.

In some embodiments, the pharmaceutical composition comprises a lubricant. In certain embodiments, the lubricant is selected from the group consisting of sodium stearyl fumarate, magnesium stearate, stearic acid, glyceryl behenate, and combinations thereof.

In further embodiments, the lubricant is magnesium stearate.

In some embodiments, the pharmaceutical composition comprises about 0.5% (w/w) to about 1.5% (w/w) lubricant. In some embodiments, the pharmaceutical composition comprises about 0.75% (w/w) to about 1.25% (w/w) lubricant. In some embodiments, the pharmaceutical composition comprises about 0.8% (w/w) to about 1% (w/w) lubricant. In some embodiments, the pharmaceutical composition comprises about 0.9% (w/w) to about 1.1% (w/w) lubricant. In some embodiments, the pharmaceutical composition comprises about 0.95% (w/w) to about 1.05% (w/w) lubricant.

In some embodiments, the pharmaceutical composition comprises about 0.9% (w/w), about 0.92% (w/w), about 0.94% (w/w), about 0.96% (w/w), about 0.98% (w/w), about 1.0% (w/w), about 1.02% (w/w), about 1.04% (w/w), about 1.06% (w/w), about 1.08% (w/w) or about 1.1% (w/w) lubricant. In some embodiments, the pharmaceutical composition comprises about 0.98% (w/w), about 1.00% (w/w) or about 1.02% (w/w) lubricant.

In some embodiments, the pharmaceutical composition comprises about 1% (w/w) lubricant.

In certain embodiments, provided is a pharmaceutical composition comprising:
(a) a compound of formula (I)

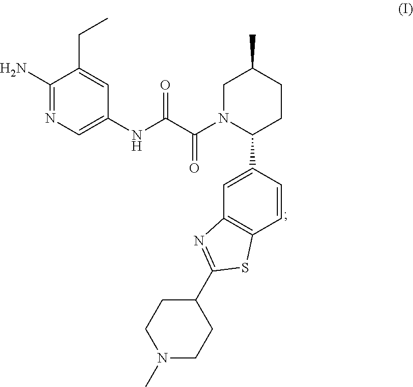 [SMILES: CCc1cc(NC(=O)C(=O)N2C[C@@H](C)CC[C@@H]2c2ccc3sc(C4CCN(C)CC4)nc3c2)cnc1N]

(b) a filler (e.g., microcrystalline cellulose);
(c) a glidant (e.g., colloidal silicon dioxide);
(d) a disintegrant (e.g., croscarmellose sodium); and
(e) a lubricant (e.g., magnesium stearate).

In some embodiments, the composition comprises a crystalline form of the compound of formula (I) described herein (e.g., Form A).

In some embodiments, the composition comprises:
(a) about 2% (w/w) to about 20% (w/w) of the compound of formula (I);
(b) about 50% (w/w) to about 90% (w/w) of a filler (e.g., microcrystalline cellulose);
(c) about 0.5% (w/w) to about 2.5% (w/w) of a glidant (e.g., colloidal silicon dioxide);
(d) about 2% (w/w) to about 6% (w/w) of a disintegrant (e.g., croscarmellose sodium);
(e) about 0.5% (w/w) to about 1.5% (w/w) of a lubricant (e.g., magnesium stearate); thereby totaling no more than 100% (w/w) of the composition.

In some embodiments, the composition comprises:
(a) about 5% (w/w) to about 15% (w/w) of the compound of formula (I);
(b) about 80% (w/w) to about 90% (w/w) of a filler (e.g., microcrystalline cellulose);
(c) about 1.4% (w/w) to about 1.6% (w/w) of a glidant (e.g., colloidal silicon dioxide);
(d) about 3.6% (w/w) to about 4.4% (w/w) of a disintegrant (e.g., croscarmellose sodium);
(e) about 0.9% (w/w) to about 1.1% (w/w) of a lubricant (e.g., magnesium stearate); thereby totaling no more than 100% (w/w) of the composition.

In some embodiments, the composition comprises:
(a) about 8% (w/w) to about 12% (w/w) of the compound of formula (I);
(b) about 80% (w/w) to about 86% (w/w) of a filler (e.g., microcrystalline cellulose);
(c) about 1.45% (w/w) to about 1.55% (w/w) of a glidant (e.g., colloidal silicon dioxide);
(d) about 3.8% (w/w) to about 4.2% (w/w) of a disintegrant (e.g., croscarmellose sodium);
(e) about 0.95% (w/w) to about 1.05% (w/w) of a lubricant (e.g., magnesium stearate); thereby totaling no more than 100% (w/w) of the composition.

In some embodiments, the composition comprises:
(a) about 10% (w/w) of the compound of formula (I);
(b) about 83.5% (w/w) of a filler (e.g., microcrystalline cellulose);
(c) about 1.5% (w/w) of a glidant (e.g., colloidal silicon dioxide);
(d) about 4% (w/w) of a disintegrant (e.g., croscarmellose sodium);
(e) about 1% (w/w) of a lubricant (e.g., magnesium stearate);
thereby totaling no more than 100% (w/w) of the composition.

The pharmaceutically acceptable excipients can be present in either the intragranular or the extragranular components of the pharmaceutical composition. In some embodiments, one or more pharmaceutically acceptable excipients are present in both the intragranular and the extragranular components.

In some embodiments, the pharmaceutical composition contains an intragranular filler selected from the fillers described herein. In certain embodiments, the intragranular filler is a microcrystalline cellulose (e.g., Avicell®). In further embodiments, the intragranular filler is a microcrystalline cellulose PH 102 (e.g., Avicel® PH 102).

In certain embodiments, the pharmaceutical composition comprises about 50% (w/w) to about 90% (w/w) intragranular filler. In certain embodiments, the pharmaceutical composition comprises about 75% (w/w) to about 86% (w/w) intragranular filler. In certain embodiments, the pharmaceutical composition comprises about 77% (w/w) to about 85% (w/w) intragranular filler. In certain embodiments, the pharmaceutical composition comprises about 80% (w/w) to about 85% (w/w) intragranular filler. In certain embodiments, the pharmaceutical composition comprises about 82% (w/w) to about 84% (w/w) intragranular filler.

In certain embodiments, the pharmaceutical composition comprises about 75% (w/w), about 76% (w/w), about 77% (w/w), about 78% (w/w), about 79% (w/w), about 80% (w/w), about 81% (w/w), about 82% (w/w), about 83% (w/w), about 83.5% (w/w), about 84% (w/w), about 85% (w/w), about 86% (w/w) intragranular filler. In certain embodiments, the pharmaceutical composition comprises about 80% (w/w), about 81% (w/w), about 82% (w/w), about 83% (w/w), about 83.5% (w/w), about 84% (w/w), about 85% (w/w), about 86% (w/w) intragranular filler.

In certain embodiments, the pharmaceutical composition comprises about 83.5% (w/w) intragranular filler.

In some embodiments, the pharmaceutical composition comprises an intragranular glidant selected from the glidants described herein. In some embodiments, the intragranular glidant is colloidal silicon dioxide (e.g., Aerosil® 200, CAB-O-SIL® M-5P).

In some embodiments, the pharmaceutical composition comprises about 0.75% (w/w) to about 1.25% (w/w) intragranular glidant. In some embodiments, the pharmaceutical composition comprises about 0.8% (w/w) to about 1.2% (w/w) intragranular glidant. In some embodiments, the pharmaceutical composition comprises about 0.85% (w/w) to about 1.15% (w/w) intragranular glidant. In some embodiments, the pharmaceutical composition comprises about 0.9% (w/w) to about 1.1% (w/w) intragranular glidant. In some embodiments, the pharmaceutical composition comprises about 0.95% (w/w) to about 1.05% (w/w) intragranular glidant. In some embodiments, the pharmaceutical composition comprises about 0.97% (w/w) to about 1.3% (w/w) intragranular glidant.

In some embodiments, the pharmaceutical composition comprises about 0.9% (w/w), about 0.91% (w/w), about 0.92% (w/w), about 0.93% (w/w), about 0.94% (w/w), about 0.95% (w/w), about 0.96% (w/w), about 0.97% (w/w), about 0.98% (w/w), about 0.99% (w/w), about 1% (w/w), about 1.01% (w/w), about 1.02% (w/w), about 1.03% (w/w), about 1.04% (w/w), about 1.05% (w/w), about 1.06% (w/w), about 1.07% (w/w), about 1.08% (w/w), about 1.09% (w/w) or about 1.1% (w/w) intragranular glidant.

In some embodiments, the pharmaceutical composition comprises about 1% (w/w) intragranular glidant.

In some embodiments, the pharmaceutical composition comprises an extragranular glidant selected from the glidants described herein. In some embodiments, the extragranular glidant is colloidal silicon dioxide (e.g., Aerosil® 200, CAB-O-SIL® M-5P).

In some embodiments, the pharmaceutical composition comprises about 0.25% (w/w) to about 0.75% (w/w) extragranular glidant. In some embodiments, the pharmaceutical composition comprises about 0.3% (w/w) to about 0.7% (w/w) extragranular glidant. In some embodiments, the pharmaceutical composition comprises about 0.4% (w/w) to about 0.6% (w/w) extragranular glidant. In some embodiments, the pharmaceutical composition comprises about 0.45% (w/w) to about 0.55% (w/w) extragranular glidant.

In some embodiments, the pharmaceutical composition comprises about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) extragranular glidant. In some embodiments, the pharmaceutical composition comprises about 0.48% (w/w), about 0.49% (w/w), about 0.5% (w/w), about 0.51% (w/w) or about 0.52% (w/w) extragranular glidant.

In some embodiments, the pharmaceutical composition comprises about 0.5% (w/w) extragranular glidant.

In some embodiments, the pharmaceutical composition comprises an intragranular disintegrant selected from the disintegrants described herein. In certain embodiments, the intragranular disintegrant is croscarmellose sodium.

In some embodiments, the pharmaceutical composition comprises about 1% (w/w) to about 3% (w/w) intragranular disintegrant. In some embodiments, the pharmaceutical composition comprises about 1.5% (w/w) to about 2.5% (w/w) intragranular disintegrant.

In some embodiments, the pharmaceutical composition comprises about 1.8% (w/w) to about 2.2% (w/w) intragranular disintegrant. In some embodiments, the pharmaceutical composition comprises about 1.9% (w/w) to about 2.1% (w/w) intragranular disintegrant.

In some embodiments, the pharmaceutical composition comprises about 1.92% (w/w) to about 2.08% (w/w) intragranular disintegrant. In some embodiments, the pharmaceutical composition comprises about 1.95% (w/w) to about 2.05% (w/w) intragranular disintegrant.

In some embodiments, the pharmaceutical composition comprises about 1.88% (w/w), about 1.92% (w/w), about 1.96% (w/w), about 2% (w/w), about 2.04% (w/w), about 2.08% (w/w), about 2.12% (w/w), about 2.16% (w/w) or about 2.2% (w/w) intragranular disintegrant. In some embodiments, the pharmaceutical composition comprises about 1.92% (w/w), about 1.93% (w/w), about 1.94% (w/w), about 1.95% (w/w), about 1.96% (w/w), about 1.97% (w/w), about 1.98% (w/w), about 1.99% (w/w), about 2% (w/w), about 2.01% (w/w), about 2.02% (w/w), about 2.03% (w/w), about 2.04% (w/w), about 2.05% (w/w), about 2.06% (w/w), about 2.07% (w/w), about 2.08% (w/w), about 2.09% (w/w), about 2.1% (w/w), about 2.11% (w/w) or about 2.12% (w/w) intragranular disintegrant.

In some embodiments, the pharmaceutical composition comprises about 2% (w/w) intragranular disintegrant.

In some embodiments, the pharmaceutical composition comprises an extragranular disintegrant selected from the disintegrants described herein. In certain embodiments, the extragranular disintegrant is croscarmellose sodium.

In some embodiments, the pharmaceutical composition comprises about 1% (w/w) to about 3% (w/w) extragranular disintegrant. In some embodiments, the pharmaceutical composition comprises about 1.5% (w/w) to about 2.5% (w/w) extragranular disintegrant.

In some embodiments, the pharmaceutical composition comprises about 1.8% (w/w) to about 2.2% (w/w) extragranular disintegrant. In some embodiments, the pharmaceutical composition comprises about 1.9% (w/w) to about 2.1% (w/w) extragranular disintegrant.

In some embodiments, the pharmaceutical composition comprises about 1.92% (w/w) to about 2.08% (w/w) extragranular disintegrant. In some embodiments, the pharmaceutical composition comprises about 1.95% (w/w) to about 2.05% (w/w) extragranular disintegrant.

In some embodiments, the pharmaceutical composition comprises about 1.88% (w/w), about 1.92% (w/w), about 1.96% (w/w), about 2% (w/w), about 2.04% (w/w), about 2.08% (w/w), about 2.12% (w/w), about 2.16% (w/w) or about 2.2% (w/w) extragranular disintegrant. In some embodiments, the pharmaceutical composition comprises about 1.92% (w/w), about 1.93% (w/w), about 1.94% (w/w), about 1.95% (w/w), about 1.96% (w/w), about 1.97% (w/w), about 1.98% (w/w), about 1.99% (w/w), about 2% (w/w), about 2.01% (w/w), about 2.02% (w/w), about 2.03% (w/w), about 2.04% (w/w), about 2.05% (w/w), about 2.06% (w/w), about 2.07% (w/w), about 2.08% (w/w), about 2.09% (w/w), about 2.1% (w/w), about 2.11% (w/w) or about 2.12% (w/w) extragranular disintegrant.

In some embodiments, the pharmaceutical composition comprises about 2% (w/w) extragranular disintegrant.

In some embodiments, the pharmaceutical composition comprises an intragranular lubricant selected from the lubricants described herein. In certain embodiments, the intragranular lubricant is magnesium stearate.

In some embodiments, the pharmaceutical composition comprises about 0.25% (w/w) to about 0.75% (w/w) intragranular lubricant. In some embodiments, the pharmaceutical composition comprises about 0.3% (w/w) to about 0.7% (w/w) intragranular lubricant. In some embodiments, the pharmaceutical composition comprises about 0.35% (w/w) to about 0.65% (w/w) intragranular lubricant. In some embodiments, the pharmaceutical composition comprises about 0.4% (w/w) to about 0.6% (w/w) intragranular lubricant. In some embodiments, the pharmaceutical composition comprises about 0.45% (w/w) to about 0.55% (w/w) intragranular lubricant. In some embodiments, the pharmaceutical composition comprises about 0.47% (w/w) to about 0.53% (w/w) intragranular lubricant. In some embodiments, the pharmaceutical composition comprises about 0.48% (w/w) to about 0.52% (w/w) intragranular lubricant.

In some embodiments, the pharmaceutical composition comprises about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) intragranular lubricant. In some embodiments, the pharmaceutical composition comprises about 0.48% (w/w), about 0.49% (w/w), about 0.5% (w/w), about 0.51% (w/w) or about 0.52% (w/w) intragranular lubricant.

In some embodiments, the pharmaceutical composition comprises about 0.5% (w/w) intragranular lubricant.

In some embodiments, the pharmaceutical composition comprises an extragranular lubricant selected from the lubricants described herein. In certain embodiments, the extragranular lubricant is magnesium stearate.

In some embodiments, the pharmaceutical composition comprises about 0.25% (w/w) to about 0.75% (w/w) extragranular lubricant. In some embodiments, the pharmaceutical composition comprises about 0.3% (w/w) to about 0.7% (w/w) extragranular lubricant. In some embodiments, the pharmaceutical composition comprises about 0.35% (w/w) to about 0.65% (w/w) extragranular lubricant. In some embodiments, the pharmaceutical composition comprises about 0.4% (w/w) to about 0.6% (w/w) extragranular lubricant. In some embodiments, the pharmaceutical composition comprises about 0.45% (w/w) to about 0.55% (w/w) extragranular lubricant. In some embodiments, the pharmaceutical composition comprises about 0.47% (w/w) to about 0.53% (w/w) extragranular lubricant. In some embodiments, the pharmaceutical composition comprises about 0.48% (w/w) to about 0.52% (w/w) extragranular lubricant.

In some embodiments, the pharmaceutical composition comprises about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) extragranular lubricant. In some embodiments, the pharmaceutical composition comprises about 0.48% (w/w), about 0.49% (w/w), about 0.5% (w/w), about 0.51% (w/w) or about 0.52% (w/w) extragranular lubricant.

In some embodiments, the pharmaceutical composition comprises about 0.5% (w/w) extragranular lubricant.

In some embodiments, provided is a pharmaceutical composition comprising:
(a) a compound of formula (I)

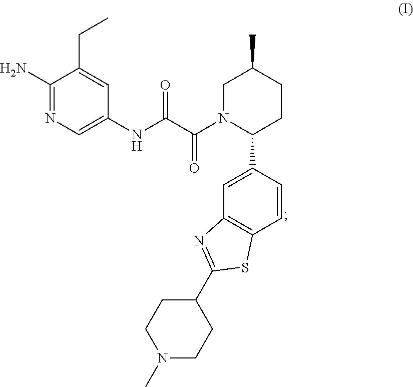 [SMILES: CCc1cc(NC(=O)C(=O)N2C[C@@H](C)CC[C@@H]2c2ccc3sc(C4CCN(C)CC4)nc3c2)cnc1N]

(b) an intragranular filler (e.g., microcrystalline cellulose);
(c) an intragranular glidant (e.g., colloidal silicon dioxide);
(d) an intragranular disintegrant (e.g., croscarmellose sodium);
(e) an intragranular lubricant (e.g., magnesium stearate);
(f) an extragranular glidant (e.g., colloidal silicon dioxide);
(g) an extragranular disintegrant (e.g., croscarmellose sodium); and
(h) an extragranular lubricant (e.g., magnesium stearate).

In some embodiments, provided is a pharmaceutical composition comprising:
(a) a crystalline form of a compound of formula (I) (e.g., crystalline form A as described herein)

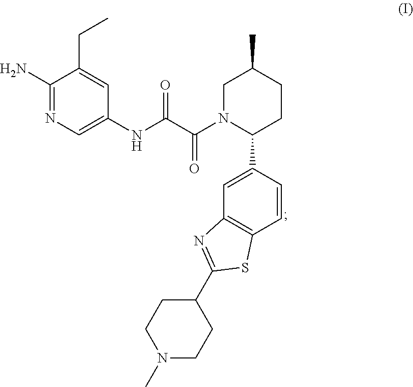 [SMILES: CCc1cc(NC(=O)C(=O)N2C[C@@H](C)CC[C@@H]2c2ccc3sc(C4CCN(C)CC4)nc3c2)cnc1N]

(b) an intragranular filler (e.g., microcrystalline cellulose);
(c) an intragranular glidant (e.g., colloidal silicon dioxide);
(d) an intragranular disintegrant (e.g., croscarmellose sodium);
(e) an intragranular lubricant (e.g., magnesium stearate);
(f) an extragranular glidant (e.g., colloidal silicon dioxide);
(g) an extragranular disintegrant (e.g., croscarmellose sodium); and
(h) an extragranular lubricant (e.g., magnesium stearate).

In some embodiments, the pharmaceutical composition comprises:
(a) about 2% (w/w) to about 20% (w/w) of the compound of formula (I) (e.g., crystalline form A);
(b) about 50% (w/w) to about 90% (w/w) of an intragranular filler (e.g., microcrystalline cellulose);
(c) about 0.75% (w/w) to about 1.25% (w/w) of an intragranular glidant (e.g., colloidal silicon dioxide);
(d) about 1% (w/w) to about 3% (w/w) of an intragranular disintegrant (e.g., croscarmellose sodium);
(e) about 0.25% (w/w) to about 0.75% (w/w) of an intragranular lubricant (e.g., magnesium stearate);
(f) about 0.25% (w/w) to about 0.75% (w/w) of an extragranular glidant (e.g., colloidal silicon dioxide);
(g) about 1% (w/w) to about 3% (w/w) of an extragranular disintegrant (e.g., croscarmellose sodium); and
(h) about 0.25% (w/w) to about 0.75% (w/w) of an extragranular lubricant (e.g., magnesium stearate);
thereby totaling no more than 100% (w/w) of the composition.

In some embodiments, the pharmaceutical composition comprises:
(a) about 5% (w/w) to about 15% (w/w) of the compound of formula (I) (e.g., crystalline form A);
(b) about 77% (w/w) to about 85% (w/w) of an intragranular filler (e.g., microcrystalline cellulose);
(c) about 0.9% (w/w) to about 1.1% (w/w) of an intragranular glidant (e.g., colloidal silicon dioxide);
(d) about 1.8% (w/w) to about 2.2% (w/w) of an intragranular disintegrant (e.g., croscarmellose sodium);
(e) about 0.9% (w/w) to about 1.1% (w/w) of an intragranular lubricant (e.g., magnesium stearate);
(f) about 0.4% (w/w) to about 0.6% (w/w) of an extragranular glidant (e.g., colloidal silicon dioxide);
(g) about 1.8% (w/w) to about 2.2% (w/w) of an extragranular disintegrant (e.g., croscarmellose sodium); and
(h) about 0.4% (w/w) to about 0.6% (w/w) of an extragranular lubricant (e.g., magnesium stearate);
thereby totaling no more than 100% (w/w) of the composition.

In some embodiments, the pharmaceutical composition comprises:
(a) about 8% (w/w) to about 12% (w/w) of the compound of formula (I) (e.g., crystalline form A);
(b) about 80% (w/w) to about 85% (w/w) of an intragranular filler (e.g., microcrystalline cellulose);
(c) about 0.95% (w/w) to about 1.05% (w/w) of an intragranular glidant (e.g., colloidal silicon dioxide);
(d) about 1.9% (w/w) to about 2.1% (w/w) of an intragranular disintegrant (e.g., croscarmellose sodium);
(e) about 0.95% (w/w) to about 1.05% (w/w) of an intragranular lubricant (e.g., magnesium stearate);
(f) about 0.45% (w/w) to about 0.55% (w/w) of an extragranular glidant (e.g., colloidal silicon dioxide);
(g) about 1.9% (w/w) to about 2.1% (w/w) of an extragranular disintegrant (e.g., croscarmellose sodium); and
(h) about 0.45% (w/w) to about 0.55% (w/w) of an extragranular lubricant (e.g., magnesium stearate);
thereby totaling no more than 100% (w/w) of the composition.

In some embodiments, the pharmaceutical composition comprises:
(a) about 10.0% (w/w) of the compound of formula (I) (e.g., crystalline form A);
(b) about 83.5% (w/w) of an intragranular filler (e.g., microcrystalline cellulose);
(c) about 1.0% (w/w) of an intragranular glidant (e.g., colloidal silicon dioxide);
(d) about 2% (w/w) of an intragranular disintegrant (e.g., croscarmellose sodium);
(e) about 0.5% (w/w) of an intragranular lubricant (e.g., magnesium stearate);
(f) about 0.5% (w/w) of an extragranular glidant (e.g., colloidal silicon dioxide);
(g) about 2% (w/w) of an extragranular disintegrant (e.g., croscarmellose sodium); and
(h) about 0.5% (w/w) of an extragranular lubricant (e.g., magnesium stearate); thereby totaling no more than 100% (w/w) of the composition.

In some embodiments, totaling no more than 100% (w/w) of the composition means “totaling 100% (w/w) of the composition”.

### Dosage Forms

In some embodiments, provided are dosage forms comprising a pharmaceutical composition described herein.

In some embodiments, provided are dosage forms intended for oral administration comprising a pharmaceutical composition described herein.

In certain embodiments, the dosage form is selected from the group consisting of a powder, a sachet, a stickpack, a capsule, a minitab, and a tablet.

In certain embodiments, the dosage form is a tablet.

In some embodiments, the total weight of the pharmaceutical composition in the dosage form is about 50 mg to 1000 mg.

In some embodiments, the total weight of the pharmaceutical composition in the dosage form is about 100 mg to 750 mg.

In some embodiments, the total weight of the pharmaceutical composition in the dosage form is about 50 mg to 150 mg.

In some embodiments, the total weight of the pharmaceutical composition in the dosage form is about 50 mg, about 60 mg, about 70 mg, about 80 mg, about 90 mg, about 100 mg, about 110 mg, about 120 mg, about 130 mg, about 140 mg or about 150 mg.

In some embodiments, the total weight of the pharmaceutical composition in the dosage form is about 80 mg, about 90 mg, about 100 mg, about 110 mg, or about 120 mg.

In some embodiments, the total weight of the pharmaceutical composition in the dosage form is about 100 mg.

In some embodiments, the total weight of the pharmaceutical composition in the dosage form is about 300 mg to 700 mg. In some embodiments, the total weight of the pharmaceutical composition in the dosage form is about 400 mg to 600 mg.

In some embodiments, the total weight of the pharmaceutical composition in the dosage form is about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg.

In some embodiments, the total weight of the pharmaceutical composition in the dosage form is about 450 mg, about 460 mg, about 480 mg, about 500 mg, about 520 mg, about 540 mg, or about 560 mg.

In some embodiments, the total weight of the pharmaceutical composition in the dosage form is about 500 mg.

In some embodiments, the total weight of the pharmaceutical composition in the dosage form is about 100 mg or about 500 mg.

In some embodiments, the composition comprises about 5 mg to about 200 mg of a compound of formula (I). In some embodiments, the dosage form comprises about 5 mg to about 100 mg of the compound of formula (I). In some embodiments, the dosage form comprises about 10 mg to about 100 mg of the compound of formula (I). In some embodiments, the dosage form comprises about 10 mg to about 50 mg of the compound of formula (I.

In some embodiments, the dosage form comprises about 5 mg to about 20 mg, about 20 mg to about 40 mg, about 40 mg to about 60 mg, about 60 mg to about 80 mg or about 80 to about 100 mg of the compound of formula (I). In some embodiments, the dosage form comprises about 5 mg to about 20 mg or about 40 mg to about 60 mg of the compound of formula (I). In some embodiments, the dosage form comprises about 5 mg to about 15 mg of the compound of formula (I). In some embodiments, the dosage form comprises about 8 mg to about 12 mg of the compound of formula (I). In some embodiments, the dosage form comprises about 9 mg to about 11 mg of the compound of formula (I).

In some embodiments, the dosage form comprises about 5 mg, about 6 mg, about 7 mg, 8 mg, about 9 mg, about 10 mg, about 11 mg, about 12 mg, about 13 mg, about 14 mg or about 15 mg of the compound of formula (I).

In some embodiments, the dosage form comprises about 10 mg of the compound of formula (I).

In some embodiments, the dosage form comprises about 40 mg to about 60 mg of the compound of formula (I). In some embodiments, the dosage form comprises about 45 mg to about 55 mg of the compound of formula (I). In some embodiments, the dosage form comprises about 47 mg to about 53 mg of the compound of formula (I).

In some embodiments, the dosage form comprises about 49 mg to about 51 mg of the compound of formula (I). In some embodiments, the dosage form comprises about 45 mg, about 46 mg, about 47 mg, about 48 mg, about 49 mg, about 50 mg, about 51 mg, about 52 mg, about 53 mg, about 54 mg or about 55 mg of the compound of formula (I).

In some embodiments, the dosage form comprises about 50 mg of the compound of formula (I).

In some embodiments, the dosage form comprises about 5 mg, about 6 mg, about 7 mg, about 8 mg, about 9 mg, about 10 mg, about 11 mg, about 12 mg, about 13 mg, about 14 mg, about 15 mg, about 45 mg, about 46 mg, about 47 mg, about 48 mg, about 49 mg, about 50 mg, about 51 mg, about 52 mg, about 53 mg, about 54 mg or about 55 mg of the compound of formula (I).

In some embodiments, the dosage form comprises about 8 mg, about 9 mg, about 10 mg, about 11 mg, about 12 mg, about 48 mg, about 49 mg, about 50 mg, about 51 mg or about 52 mg of the compound of formula (I).

In certain embodiments, the dosage form comprises about 10 mg or about 50 mg of the compound of formula (I).

In some embodiments, the dosage form comprises 10 mg Compound of formula (I), 83.5 mg intragranular microcrystalline cellulose, 1 mg intragranular colloidal silicon dioxide (e.g., Aerosil® 200, CAB-O-SIL® M-5P), 2 mg intragranular croscarmellose sodium (Ac-Di-Sol®), 0.5 mg intragranular magnesium stearate, 0.5 mg extragranular colloidal silicon dioxide (e.g., Aerosil® 200, CAB-O-SIL® M-5P), 2 mg extragranular croscarmellose sodium (Ac-Di-Sol®) and 0.5 mg extragranular magnesium stearate.

In some embodiments, the dosage form comprises 50 mg Compound of formula (I), 417.5 mg intragranular microcrystalline cellulose, 5 mg intragranular colloidal silicon dioxide (e.g., Aerosil® 200, CAB-O-SIL® M-5P), 10 mg intragranular croscarmellose sodium (Ac-Di-Sol®), 2.5 mg intragranular magnesium stearate, 2.5 mg extragranular colloidal silicon dioxide (e.g., Aerosil® 200, CAB-O-SIL® M-5P), 10 mg extragranular croscarmellose sodium (Ac-Di-Sol®) and 2.5 mg extragranular magnesium stearate.

In certain embodiments, the tablet further comprises a coating. In certain embodiments, the coating is selected from the group consisting of a film forming polymer, a plasticizer, and combinations thereof. In certain embodiments, the film forming polymer is selected from the group consisting of a hypromellose, an ethylcellulose, cellulose acetate, a polyvinylpyrrolidone, a polyvinyl alcohol, a polyacrylate, and combinations thereof. In certain embodiments, the plasticizer is selected from the group consisting of triacetin, polyethylene glycol, propylene glycol, and combinations thereof. In certain embodiments, the coating comprises polyvinyl alcohol.

In certain embodiments, the coating comprises a colorant selected from the group consisting of titanium dioxide, an aluminum lake, an iron oxide, carbon black, and combinations thereof. In some embodiments, the colorant is titanium dioxide.

### Methods of Making

In some embodiments, provided are processes for preparing the pharmaceutical compositions described herein, for example, comprising:
(a) blending the compound of formula (I) with one or more pharmaceutically acceptable excipients to obtain a blend;
(b) granulating the blend to obtain granules;
(c) milling the granules to obtain an intragranular phase; and
(d) blending the intragranular phase with one or more extragranular pharmaceutical excipients to obtain the pharmaceutical composition.

In another aspect, provided are processes for preparing the dosage forms described herein, for example, comprising:
(a) blending the compound of formula (I) with one or more pharmaceutically acceptable excipients to obtain a blend;
(b) granulating the blend to obtain granules;
(c) milling the granules to obtain an intragranular phase; and
(d) blending the intragranular phase with one or more extragranular pharmaceutical excipients to obtain the pharmaceutical composition (pharmaceutical blend).
(e) compressing the pharmaceutical blend into a tablet.

In certain embodiments, in step (a), the one or more pharmaceutically acceptable excipients is selected from the group consisting of a filler, a disintegrant, a binder, a wetting agent, a lubricant, a glidant, and combinations thereof.

In certain embodiments, in step (a), the compound of formula (I) is blended with a filler, a disintegrant, a lubricant, and a glidant.

In certain embodiments, in step (a), the filler is microcrystalline cellulose.

In some embodiments, in step (a), the disintegrant is croscarmellose sodium.

In certain embodiments, in step (a), the glidant is colloidal silicon dioxide.

In certain embodiments, in step (a), the lubricant is magnesium stearate.

In certain embodiments, in step (d), the one or more extragranular excipients is selected from the group consisting of a disintegrant, a lubricant, a glidant, and combinations thereof.

In some embodiments, in step (d), the intragranular phase is blended with a disintegrant, a lubricant, and a glidant.

In certain embodiments, in step (d), the disintegrant is croscarmellose sodium.

In some embodiments, in step (d), the glidant is colloidal silicon dioxide.

In certain embodiments, in step (d), the lubricant is magnesium stearate.

In certain embodiments, granulating the blend to obtain granules comprises a dry granulation process step. In certain embodiments, granulating the blend to obtain granules comprises a wet granulation process step.

In certain embodiments, the tablet comprises a coating. In certain embodiments, the coating comprises one or more film-forming polymers selected from the group consisting of a hypromellose, an ethylcellulose, a polyvinylpyrrolidone, a polyacrylate, a plasticizer, and combinations thereof. In further embodiments, the coating comprises a polyvinyl alcohol.

In certain embodiments, the coating comprises a colorant selected from the group consisting of titanium dioxide, an aluminum lake, an iron oxide, carbon black, and combinations thereof. In some embodiments, the colorant is titanium dioxide.

In some embodiments, provided is a pharmaceutical composition described herein prepared by the processes described herein.

### Methods of Use and Treatment

Treatment of MTAP-Deficient and/or MTA-Accumulating Proliferation Disorders

5-Methylthioadenosine phosphorylase (MTAP) catalyzes the reversible phosphorylation of S-methyl-5′-thioadenosine (MTA) to adenine and 5-methylthioribose-1-phosphate. MTAP-deletion is a common genetic event in human cancer. MTAP deletion frequency in a subset of human cancers is described in Cerami et al., Cancer Discov. (2012); 2(5):401-4; Gao et al., Sci Signal. (2013); 6(269): p 11; and Lee et al., Nat. Gen. (2014) 46(11):1227-32. For example, more than 50% of malignant peripheral nerve sheath tumor (MPNST) have deletions in MTAP (Lee et al., Nat. Gen. (2014)). Other cancers with high MTAP deletion frequencies are glioblastoma (GBM), mesothelioma, bladder cancer, pancreatic cancer, esophageal cancer, squamous lung cancer, melanoma, diffuse large B cell lymphoma (DLBCL), head and neck cancer, cholangiocarcinoma, lung adenoma, sarcoma, stomach cancer, glioma, adrenal carcinoma, thymoma, breast cancer, liver cancer, ovarian cancer, renal papillary cancer, uterine cancer, prostate cancer, and renal clear cell cancer. MTAP deletion in cells is one of the mechanisms that leads to MTAP-deficiency, increased intracellular MTA accumulation, and confers enhanced dependency on the protein arginine methyltransferase 5 (PRMT5) in cancer cells. Other mechanisms leading to MTAP deficiency include, inter alia, MTAP translocations and MTAP epigenetic silencing which could also lead to MTAP-null and/or MTAP deficient tumors. PRMT5 mediates the formation of symmetric dimethylarginine (SDMA); thus, the PRMT5 activity can be assessed by measuring the SDMA levels using the antibody against an SDMA or SDMA modified polypeptide.

In some embodiments, provided are methods of treating human or animal subjects having or having been diagnosed with an MTAP-deficiency-related and/or MTA-accumulating proliferative disorder (e.g., cancer) comprising administering to the subject in need thereof a therapeutically effective amount of a compound of the present disclosure (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein.

In some embodiments, provided are methods for treating an MTAP-deficiency-related and/or MTA-accumulating proliferative disorder (e.g., cancer) in a subject in need thereof comprising administering to the subject an effective amount (e.g., a therapeutically effective amount) of a compound of the present disclosure (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein.

In some embodiments, provided are methods of treating human or animal subjects having or having been diagnosed with an MTAP-deficiency-related and/or MTA-accumulating proliferative disorder (e.g., cancer) comprising administering to the subject in need thereof a therapeutically effective amount of pharmaceutical composition of the present disclosure. In some embodiment, the method comprises administering to the subject a dosage form of the present disclosure. In one embodiment, the compound or composition is administered in combination with a second therapeutic agent.

In some embodiments, provided are methods of treating an MTAP-deficiency-related and/or MTA-accumulating proliferative disorder (e.g., cancer) in a subject in need thereof comprising administering to the subject a therapeutically effective amount of pharmaceutical composition of the present disclosure. In some embodiment, the method comprises administering to the subject a dosage form of the present disclosure. In one embodiment, the compound or composition is administered in combination with a second therapeutic agent.

In some embodiments, provided are compounds and compositions (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein) for use in the manufacturing of a medicament. In some embodiments, provided are compounds and compositions (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein) for use in the manufacturing of a medicament for treating human or animal subjects having or having been diagnosed with an MTAP-deficiency-related and/or MTA-accumulating proliferative disorder (e.g., cancer).

In some embodiments, provided are compounds and compositions (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein) for use in the manufacturing of a medicament for treating an MTAP-deficiency-related and/or MTA-accumulating proliferative disorder (e.g., cancer) in a subject in need thereof.

In some embodiments, provided are compounds and compositions (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein) for use in the manufacturing of a medicament for treating human or animal subjects having or having been diagnosed with an MTAP-deficiency-related and/or MTA-accumulating proliferative disorder (e.g., cancer). In an embodiment, the medicament is configured for administration in combination with a second therapeutic agent. In some embodiments, the medicament comprises a therapeutically effective amount of the compound or composition (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein).

In some embodiments, provided are compounds and compositions (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein) for use in a method of treating human or animal subjects having or having been diagnosed with an MTAP-deficiency-related and/or MTA-accumulating proliferative disorder (e.g., cancer).

In some embodiments, provided are compounds and compositions (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein) for use in a method of treating an MTAP-deficiency-related and/or MTA-accumulating proliferative disorder (e.g., cancer) in a subject in need thereof.

In some embodiments, provided are compounds and compositions (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein) for use in a method of treating human or animal subjects having or having been diagnosed with an MTAP-deficiency-related and/or MTA-accumulating proliferative disorder (e.g., cancer). In an embodiment, the method comprises administration of the compound or composition in combination with a second therapeutic agent. In some embodiments, the method comprises administering to a patient in need thereof a therapeutically effective amount of the compound or composition (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein).

In some embodiments, provided are uses of compounds and compositions (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein) for treating human or animal subjects having or having been diagnosed with an MTAP-deficiency-related and/or MTA-accumulating proliferative disorder (e.g., cancer).

In some embodiments, provided are uses of compounds and compositions (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein) for treating an MTAP-deficiency-related and/or MTA-accumulating proliferative disorder (e.g., cancer) in a subject in need thereof.

In some embodiments, provided are uses of compounds and compositions (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein) for treating human or animal subjects having or having been diagnosed with an MTAP-deficiency-related and/or MTA-accumulating proliferative disorder (e.g., cancer). In an embodiment, the use comprises administration of the compound or composition in combination with a second therapeutic agent. In some embodiments, the use comprises administration to a patient in need thereof of a therapeutically effective amount of the compound or composition (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein).

In some embodiments, provided are uses of compounds and compositions (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein) in the manufacturing of a medicament for treating human or animal subjects having or having been diagnosed with an MTAP-deficiency-related and/or MTA-accumulating proliferative disorder (e.g., cancer).

In some embodiments, provided are uses of compounds and compositions (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein) in the manufacturing of a medicament for treating an MTAP-deficiency-related and/or MTA-accumulating proliferative disorder (e.g., cancer) in a subject in need thereof.

In some embodiments, provided are uses of compounds and compositions (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein) in the manufacturing of a medicament for treating human or animal subjects having or having been diagnosed with an MTAP-deficiency-related and/or MTA-accumulating proliferative disorder (e.g., cancer). In an embodiment, the medicament is configured for administration in combination with a second therapeutic agent. In some embodiments, the medicament comprises a therapeutically effective amount of the compound or composition (e.g., a crystalline form of a compound of Formula (I), crystalline Form A of the compound of formula (I)), a pharmaceutical composition comprising a compound of formula (I) or a pharmaceutically acceptable salt thereof as described herein, or a dosage form as described herein).

In certain embodiments, the disease is an MTAP-deficient and/or MTA-accumulating cancer.

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma, e.g., pancreatic ductal adenocarcinoma (PDAC)), mesothelioma, melanoma, non-small cell lung cancer (NSCLC; e.g., lung squamous or lung adenocarcinoma), astrocytoma, undifferentiated pleomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the cancer is a CNS cancer. In some embodiments, the cancer is not a CNS cancer.

In some embodiments, the cancer is a cancer selected from the group of glioblastoma, glioblastoma multiforme, urothelial carcinoma, malignant peripheral nerve sheath tumor (MPNST), cholangiocarcinoma, NSCLC (e.g., squamous and adenocarcinoma), bladder cancer, pancreatic cancer (e.g., pancreatic ductal adenocarcinoma (PDAC)), mesothelioma, colorectal cancer, chronic myelogenous leukemia (CML), and diffuse large B-cell lymphoma (DLBCL).

In some embodiments, the cancer is a cancer selected from the group of glioblastoma, glioblastoma multiforme, cholangiocarcinoma, NSCLC (e.g., squamous and adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group consisting of glioma, glioblastoma, non-small cell lung cancer (adenocarcinoma and squamous), mesothelioma, cholangiocarcinoma, bladder cancer, urothelial carcinoma, pancreatic cancer (e.g., pancreatic ductal adenocarcinoma (PDAC)) and malignant peripheral nerve sheath tumor.

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) and mesothelioma.

In some embodiments, the cancer is a cancer selected from the group of cholangiocarcinoma, NSCLC (squamous), bladder cancer, pancreatic cancer (e.g., pancreatic ductal adenocarcinoma (PDAC)) and mesothelioma.

In some embodiments, the cancer is glioma. In some embodiments, the cancer is glioblastoma. In some embodiments, the cancer is glioblastoma multiforme. In some embodiments, the cancer is urothelial carcinoma, malignant peripheral nerve sheath tumor (MPNST). In some embodiments, the cancer is cholangiocarcinoma. In some embodiments, the cancer is NSCLC (e.g., lung squamous and lung adenocarcinoma). In some embodiments, the cancer is squamous NSCLC. In some embodiments, the cancer is NSCLC adenocarcinoma. In some embodiments, the cancer is bladder cancer (e.g., bladder urothelial carcinoma). In some embodiments, the cancer is pancreatic cancer (e.g., pancreatic ductal adenocarcinoma (PDAC)). In some embodiments, the cancer is pancreatic ductal adenocarcinoma (PDAC). In some embodiments, the cancer is mesothelioma. In some embodiments, the cancer is colorectal cancer. In some embodiments, the cancer is chronic myelogenous leukemia (CML). In some embodiments, the cancer is diffuse large B-cell lymphoma (DLBCL). In some embodiments, the cancer is malignant peripheral nerve sheath tumors (MPNST). In some embodiments, the cancer is esophageal cancer (e.g., esophageal squamous cell carcinoma or esophageal adenocarcinoma).

In some embodiments, the cancer is melanoma. In some embodiments, the cancer is astrocytoma. In some embodiments, the cancer is undifferentiated pleomorphic sarcoma. In some embodiments, the cancer is leukemia. In some embodiments, the cancer is head and neck cancer. In some embodiments, the cancer is stomach adenocarcinoma. In some embodiments, the cancer is myxofibrosarcoma. In some embodiments, the cancer is cholangiosarcoma. In some embodiments, the cancer is cancer of the brain. In some embodiments, the cancer is stomach cancer. In some embodiments, the cancer is kidney cancer. In some embodiments, the cancer is breast cancer. In some embodiments, the cancer is endometrial cancer. In some embodiments, the cancer is urinary tract cancer. In some embodiments, the cancer is liver cancer. In some embodiments, the cancer is soft tissue cancer. In some embodiments, the cancer is pleura cancer. In some embodiments, the cancer is large intestine cancer. In some embodiments, the cancer is sarcoma.

In some embodiments, the cancer is an MTAP-deficient and/or MTA-accumulating glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma, e.g., pancreatic ductal adenocarcinoma (PDAC)), mesothelioma, melanoma, non-small cell lung cancer (NSCLC; e.g., lung squamous or lung adenocarcinoma), astrocytoma, undifferentiated pleomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the cancer is a CNS cancer. In some embodiments, the cancer is not a CNS cancer.

In some embodiments, the cancer is a cancer selected from the group of glioblastoma, glioblastoma multiforme, urothelial carcinoma, malignant peripheral nerve sheath tumor (MPNST), cholangiocarcinoma, NSCLC (e.g., squamous and adenocarcinoma), bladder cancer, pancreatic cancer (e.g., pancreatic ductal adenocarcinoma (PDAC)), mesothelioma, colorectal cancer, chronic myelogenous leukemia (CML), and diffuse large B-cell lymphoma (DLBCL).

In some embodiments, the cancer is a cancer selected from the group of glioblastoma, glioblastoma multiforme, cholangiocarcinoma, NSCLC (e.g., squamous and adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group consisting of glioma, glioblastoma, non-small cell lung cancer (adenocarcinoma and squamous), mesothelioma, cholangiocarcinoma, bladder cancer, urothelial carcinoma, pancreatic cancer (e.g., pancreatic ductal adenocarcinoma (PDAC)) and malignant peripheral nerve sheath tumor.

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) and mesothelioma.

In some embodiments, the cancer is a cancer selected from the group of cholangiocarcinoma, NSCLC (squamous), bladder cancer, pancreatic cancer (e.g., pancreatic ductal adenocarcinoma (PDAC)) and mesothelioma.

In some embodiments, the cancer is glioma. In some embodiments, the cancer is glioblastoma. In some embodiments, the cancer is glioblastoma multiforme. In some embodiments, the cancer is urothelial carcinoma, malignant peripheral nerve sheath tumor (MPNST). In some embodiments, the cancer is cholangiocarcinoma. In some embodiments, the cancer is NSCLC (e.g., lung squamous and lung adenocarcinoma). In some embodiments, the cancer is squamous NSCLC. In some embodiments, the cancer is NSCLC adenocarcinoma. In some embodiments, the cancer is bladder cancer (e.g., bladder urothelial carcinoma). In some embodiments, the cancer is pancreatic cancer (e.g., pancreatic ductal adenocarcinoma (PDAC)). In some embodiments, the cancer is pancreatic ductal adenocarcinoma (PDAC). In some embodiments, the cancer is mesothelioma. In some embodiments, the cancer is colorectal cancer. In some embodiments, the cancer is chronic myelogenous leukemia (CML). In some embodiments, the cancer is diffuse large B-cell lymphoma (DLBCL). In some embodiments, the cancer is malignant peripheral nerve sheath tumors (MPNST). In some embodiments, the cancer is esophageal cancer (e.g., esophageal squamous cell carcinoma or esophageal adenocarcinoma). In some embodiments, the cancer is melanoma. In some embodiments, the cancer is astrocytoma. In some embodiments, the cancer is undifferentiated pleomorphic sarcoma. In some embodiments, the cancer is leukemia. In some embodiments, the cancer is head and neck cancer. In some embodiments, the cancer is stomach adenocarcinoma. In some embodiments, the cancer is myxofibrosarcoma. In some embodiments, the cancer is cholangiosarcoma. In some embodiments, the cancer is cancer of the brain. In some embodiments, the cancer is stomach cancer. In some embodiments, the cancer is kidney cancer. In some embodiments, the cancer is breast cancer. In some embodiments, the cancer is endometrial cancer. In some embodiments, the cancer is urinary tract cancer. In some embodiments, the cancer is liver cancer. In some embodiments, the cancer is soft tissue cancer. In some embodiments, the cancer is pleura cancer. In some embodiments, the cancer is large intestine cancer. In some embodiments, the cancer is sarcoma.

In some embodiments, the compound of formula (I), crystalline forms (e.g., crystalline form A), pharmaceutical compositions (e.g., comprising a compound of formula (I) or a pharmaceutically acceptable salt thereof) and dosage forms thereof, as described herein can be used in a method of inhibiting proliferation of MTAP-deficient cells in a subject in need thereof, the method comprising the step of administering to the subject a compound of formula (I), crystalline forms (e.g., crystalline form A), pharmaceutical compositions (e.g., comprising a compound of formula (I) or a pharmaceutically acceptable salt thereof) or dosage forms thereof, as described herein in an amount that is effective to inhibit proliferation of the MTAP-deficient cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma, e.g., pancreatic ductal adenocarcinoma (PDAC)), mesothelioma, melanoma, non-small cell lung cancer (NSCLC; e.g., lung squamous or lung adenocarcinoma), astrocytoma, undifferentiated pleomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the cancer is a CNS cancer. In some embodiments, the cancer is not a CNS cancer.

In some embodiments, the cancer is a cancer selected from the group of glioblastoma, glioblastoma multiforme, urothelial carcinoma, malignant peripheral nerve sheath tumor (MPNST), cholangiocarcinoma, NSCLC (e.g., squamous and adenocarcinoma), bladder cancer, pancreatic cancer (e.g., pancreatic ductal adenocarcinoma (PDAC)), mesothelioma, colorectal cancer, chronic myelogenous leukemia (CML), and diffuse large B-cell lymphoma (DLBCL).

In some embodiments, the cancer is a cancer selected from the group of glioblastoma, glioblastoma multiforme, cholangiocarcinoma, NSCLC (e.g., squamous and adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group consisting of glioma, glioblastoma, non-small cell lung cancer (adenocarcinoma and squamous), mesothelioma, cholangiocarcinoma, bladder cancer, urothelial carcinoma, pancreatic cancer (e.g., pancreatic ductal adenocarcinoma (PDAC)) and malignant peripheral nerve sheath tumor.

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) and mesothelioma.

In some embodiments, the cancer is a cancer selected from the group of cholangiocarcinoma, NSCLC (squamous), bladder cancer, pancreatic cancer (e.g., pancreatic ductal adenocarcinoma (PDAC)) and mesothelioma.

In some embodiments, the cancer is glioma. In some embodiments, the cancer is glioblastoma. In some embodiments, the cancer is glioblastoma multiforme. In some embodiments, the cancer is urothelial carcinoma, malignant peripheral nerve sheath tumor (MPNST). In some embodiments, the cancer is cholangiocarcinoma. In some embodiments, the cancer is NSCLC (e.g., lung squamous and lung adenocarcinoma). In some embodiments, the cancer is squamous NSCLC. In some embodiments, the cancer is NSCLC adenocarcinoma. In some embodiments, the cancer is bladder cancer (e.g., bladder urothelial carcinoma). In some embodiments, the cancer is pancreatic cancer (e.g., pancreatic ductal adenocarcinoma (PDAC)). In some embodiments, the cancer is pancreatic ductal adenocarcinoma (PDAC). In some embodiments, the cancer is mesothelioma. In some embodiments, the cancer is colorectal cancer. In some embodiments, the cancer is chronic myelogenous leukemia (CML). In some embodiments, the cancer is diffuse large B-cell lymphoma (DLBCL). In some embodiments, the cancer is malignant peripheral nerve sheath tumors (MPNST). In some embodiments, the cancer is esophageal cancer (e.g., esophageal squamous cell carcinoma or esophageal adenocarcinoma). In some embodiments, the cancer is melanoma. In some embodiments, the cancer is astrocytoma. In some embodiments, the cancer is undifferentiated pleomorphic sarcoma. In some embodiments, the cancer is leukemia. In some embodiments, the cancer is head and neck cancer. In some embodiments, the cancer is stomach adenocarcinoma. In some embodiments, the cancer is myxofibrosarcoma. In some embodiments, the cancer is cholangiosarcoma. In some embodiments, the cancer is cancer of the brain. In some embodiments, the cancer is stomach cancer. In some embodiments, the cancer is kidney cancer. In some embodiments, the cancer is breast cancer. In some embodiments, the cancer is endometrial cancer. In some embodiments, the cancer is urinary tract cancer. In some embodiments, the cancer is liver cancer. In some embodiments, the cancer is soft tissue cancer. In some embodiments, the cancer is pleura cancer. In some embodiments, the cancer is large intestine cancer. In some embodiments, the cancer is sarcoma.

In some embodiments, the compound of formula (I), crystalline forms (e.g., crystalline form A), pharmaceutical compositions (e.g., comprising a compound of formula (I) or a pharmaceutically acceptable salt thereof) or dosage forms thereof, as described herein can be used in a method of inhibiting proliferation of MTA-accumulating cells in a subject in need thereof, the method comprising the step of administering to the subject a compound of formula (I), crystalline forms (e.g., crystalline form A), pharmaceutical compositions (e.g., comprising a compound of formula (I) or a pharmaceutically acceptable salt thereof) or dosage forms thereof, as described herein in an amount that is effective to inhibit proliferation of the MTA-accumulating cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma, e.g., pancreatic ductal adenocarcinoma (PDAC)), mesothelioma, melanoma, non-small cell lung cancer (NSCLC; e.g., lung squamous or lung adenocarcinoma), astrocytoma, undifferentiated pleomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the cancer is a CNS cancer. In some embodiments, the cancer is not a CNS cancer.

In some embodiments, the cancer is a cancer selected from the group of glioblastoma, glioblastoma multiforme, urothelial carcinoma, malignant peripheral nerve sheath tumor (MPNST), cholangiocarcinoma, NSCLC (e.g., squamous and adenocarcinoma), bladder cancer, pancreatic cancer (e.g., pancreatic ductal adenocarcinoma (PDAC)), mesothelioma, colorectal cancer, chronic myelogenous leukemia (CML), and diffuse large B-cell lymphoma (DLBCL).

In some embodiments, the cancer is a cancer selected from the group of glioblastoma, glioblastoma multiforme, cholangiocarcinoma, NSCLC (e.g., squamous and adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group consisting of glioma, glioblastoma, non-small cell lung cancer (adenocarcinoma and squamous), mesothelioma, cholangiocarcinoma, bladder cancer, urothelial carcinoma, pancreatic cancer (e.g., pancreatic ductal adenocarcinoma (PDAC)) and malignant peripheral nerve sheath tumor.

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) and mesothelioma.

In some embodiments, the cancer is a cancer selected from the group of cholangiocarcinoma, NSCLC (squamous), bladder cancer, pancreatic cancer (e.g., pancreatic ductal adenocarcinoma (PDAC)) and mesothelioma.

In some embodiments, the cancer is glioma. In some embodiments, the cancer is glioblastoma. In some embodiments, the cancer is glioblastoma multiforme. In some embodiments, the cancer is urothelial carcinoma, malignant peripheral nerve sheath tumor (MPNST). In some embodiments, the cancer is cholangiocarcinoma. In some embodiments, the cancer is NSCLC (e.g., lung squamous and lung adenocarcinoma). In some embodiments, the cancer is squamous NSCLC. In some embodiments, the cancer is NSCLC adenocarcinoma. In some embodiments, the cancer is bladder cancer (e.g., bladder urothelial carcinoma). In some embodiments, the cancer is pancreatic cancer (e.g., pancreatic ductal adenocarcinoma (PDAC)). In some embodiments, the cancer is pancreatic ductal adenocarcinoma (PDAC). In some embodiments, the cancer is mesothelioma. In some embodiments, the cancer is colorectal cancer. In some embodiments, the cancer is chronic myelogenous leukemia (CML). In some embodiments, the cancer is diffuse large B-cell lymphoma (DLBCL). In some embodiments, the cancer is malignant peripheral nerve sheath tumors (MPNST). In some embodiments, the cancer is esophageal cancer (e.g., esophageal squamous cell carcinoma or esophageal adenocarcinoma). In some embodiments, the cancer is melanoma. In some embodiments, the cancer is astrocytoma. In some embodiments, the cancer is undifferentiated pleomorphic sarcoma. In some embodiments, the cancer is leukemia. In some embodiments, the cancer is head and neck cancer. In some embodiments, the cancer is stomach adenocarcinoma. In some embodiments, the cancer is myxofibrosarcoma. In some embodiments, the cancer is cholangiosarcoma. In some embodiments, the cancer is cancer of the brain. In some embodiments, the cancer is stomach cancer. In some embodiments, the cancer is kidney cancer. In some embodiments, the cancer is breast cancer. In some embodiments, the cancer is endometrial cancer. In some embodiments, the cancer is urinary tract cancer. In some embodiments, the cancer is liver cancer. In some embodiments, the cancer is soft tissue cancer. In some embodiments, the cancer is pleura cancer. In some embodiments, the cancer is large intestine cancer. In some embodiments, the cancer is sarcoma.

In some embodiments, the compound of formula (I), crystalline forms (e.g., crystalline form A), pharmaceutical compositions (e.g., comprising a compound of formula (I) or a pharmaceutically acceptable salt thereof) or dosage forms thereof, as described herein can be used in a method of inhibiting proliferation of MTAP deficient and/or MTA-accumulating cells in a subject in need thereof, the method comprising the step of administering to the subject a compound of formula (I), crystalline forms (e.g., crystalline form A), pharmaceutical compositions (e.g., comprising a compound of formula (I) or a pharmaceutically acceptable salt thereof) or dosage forms thereof, as described herein in an amount that is effective to inhibit proliferation of the MTAP deficient and/or MTA-accumulating cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma, e.g., pancreatic ductal adenocarcinoma (PDAC)), mesothelioma, melanoma, non-small cell lung cancer (NSCLC; e.g., lung squamous or lung adenocarcinoma), astrocytoma, undifferentiated pleomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the cancer is a CNS cancer. In some embodiments, the cancer is not a CNS cancer.

In some embodiments, the cancer is a cancer selected from the group of glioblastoma, glioblastoma multiforme, urothelial carcinoma, malignant peripheral nerve sheath tumor (MPNST), cholangiocarcinoma, NSCLC (e.g., squamous and adenocarcinoma), bladder cancer, pancreatic cancer (e.g., pancreatic ductal adenocarcinoma (PDAC)), mesothelioma, colorectal cancer, chronic myelogenous leukemia (CML), and diffuse large B-cell lymphoma (DLBCL).

In some embodiments, the cancer is a cancer selected from the group of glioblastoma, glioblastoma multiforme, cholangiocarcinoma, NSCLC (e.g., squamous and adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group consisting of glioma, glioblastoma, non-small cell lung cancer (adenocarcinoma and squamous), mesothelioma, cholangiocarcinoma, bladder cancer, urothelial carcinoma, pancreatic cancer (e.g., pancreatic ductal adenocarcinoma (PDAC)) and malignant peripheral nerve sheath tumor.

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) and mesothelioma.

In some embodiments, the cancer is a cancer selected from the group of cholangiocarcinoma, NSCLC (squamous), bladder cancer, pancreatic cancer (e.g., pancreatic ductal adenocarcinoma (PDAC)) and mesothelioma.

In some embodiments, the cancer is glioma. In some embodiments, the cancer is glioblastoma. In some embodiments, the cancer is glioblastoma multiforme. In some embodiments, the cancer is urothelial carcinoma, malignant peripheral nerve sheath tumor (MPNST). In some embodiments, the cancer is cholangiocarcinoma. In some embodiments, the cancer is NSCLC (e.g., lung squamous and lung adenocarcinoma). In some embodiments, the cancer is squamous NSCLC. In some embodiments, the cancer is NSCLC adenocarcinoma. In some embodiments, the cancer is bladder cancer (e.g., bladder urothelial carcinoma). In some embodiments, the cancer is pancreatic cancer (e.g., pancreatic ductal adenocarcinoma (PDAC)). In some embodiments, the cancer is pancreatic ductal adenocarcinoma (PDAC). In some embodiments, the cancer is mesothelioma. In some embodiments, the cancer is colorectal cancer. In some embodiments, the cancer is chronic myelogenous leukemia (CML). In some embodiments, the cancer is diffuse large B-cell lymphoma (DLBCL). In some embodiments, the cancer is malignant peripheral nerve sheath tumors (MPNST). In some embodiments, the cancer is esophageal cancer (e.g., esophageal squamous cell carcinoma or esophageal adenocarcinoma). In some embodiments, the cancer is melanoma. In some embodiments, the cancer is astrocytoma. In some embodiments, the cancer is undifferentiated pleomorphic sarcoma. In some embodiments, the cancer is leukemia. In some embodiments, the cancer is head and neck cancer. In some embodiments, the cancer is stomach adenocarcinoma. In some embodiments, the cancer is myxofibrosarcoma. In some embodiments, the cancer is cholangiosarcoma. In some embodiments, the cancer is cancer of the brain. In some embodiments, the cancer is stomach cancer. In some embodiments, the cancer is kidney cancer. In some embodiments, the cancer is breast cancer. In some embodiments, the cancer is endometrial cancer. In some embodiments, the cancer is urinary tract cancer. In some embodiments, the cancer is liver cancer. In some embodiments, the cancer is soft tissue cancer. In some embodiments, the cancer is pleura cancer. In some embodiments, the cancer is large intestine cancer. In some embodiments, the cancer is sarcoma.

### Combination Therapies

In some embodiments, provided are methods of treatment of MTAP-deficient and/or MTA accumulating proliferative disorders (e.g., cancers) with a compound of formula (I), crystalline forms (e.g., crystalline form A), pharmaceutical compositions (e.g., comprising a compound of formula (I) or a pharmaceutically acceptable salt thereof) or dosage forms thereof, as described herein in combination with one or more therapeutic agents.

In some embodiments, provided are methods of treatment of MTAP-deficient and/or MTA accumulating proliferative disorders (e.g., cancers) with a compound of formula (I), crystalline forms (e.g., crystalline form A), pharmaceutical compositions (e.g., comprising a compound of formula (I) or a pharmaceutically acceptable salt thereof) or dosage forms thereof, as described herein in combination with a second therapeutic agent. In some embodiments, provided are methods of treatment of MTAP-deficient and/or MTA accumulating proliferative disorders (e.g., cancers) with a compound of formula (I), crystalline forms (e.g., crystalline form A), pharmaceutical compositions (e.g., comprising a compound of formula (I) or a pharmaceutically acceptable salt thereof) or dosage forms thereof, as described herein in combination with a second therapeutic agent and a third therapeutic agent. In some embodiments, provided are methods of treatment of MTAP-deficient and/or MTA accumulating proliferative disorders (e.g., cancers) with a compound of formula (I), crystalline forms (e.g., crystalline form A), pharmaceutical compositions (e.g., comprising a compound of formula (I) or a pharmaceutically acceptable salt thereof) or dosage forms thereof, as described herein in combination with a second therapeutic agent, a third therapeutic agent, and a fourth therapeutic agent.

The term “Combination” refers to either a fixed combination in one dosage unit form, or a combined administration where a compound of formula (I), crystalline forms (e.g., crystalline form A), pharmaceutical compositions (e.g., comprising a compound of formula (I) or a pharmaceutically acceptable salt thereof) or dosage forms thereof, as described herein and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein and a combination partner, are both administered to a patient simultaneously in the form of a single entity or dosage. The term “non-fixed combination” means that the therapeutic agents, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more therapeutic agent.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times.

In certain embodiments, a compound of formula (I), crystalline forms (e.g., crystalline form A), pharmaceutical compositions (e.g., comprising a compound of formula (I) or a pharmaceutically acceptable salt thereof) or dosage forms thereof, as described herein are combined with other therapeutic agents, including, but not limited to, other anti-cancer agents, anti-allergic agents, anti-nausea agents (or anti-emetics), pain relievers, cytoprotective agents, and combinations thereof.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a general chemotherapeutic agent selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitabine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan)(Alkeran®, 6-mercaptopurine (Purinethol®), methotrexate (Folex®), mitoxantrone (Novantrone®), mylotarg, paclitaxel (Taxol®), nab—paclitaxel (Abraxane®), phoenix (Yttrium90/MX-DTPA), pentostatin, polifeprosan 20 with carmustine implant (Gliadel®), tamoxifen citrate (Nolvadex), teniposide (Vumon®), 6—thioguanine, thiotepa, tirapazamine (Tirazone®), topotecan hydrochloride for injection (Hycamptin®), vinblastine (Velban), vincristine (Oncovin®), and vinorelbine (Navelbine®).

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an EGFR-inhibitor (e.g., cetuximab, panitumumab, erlotinib, gefitinib, afatinib, aumolertinib, olmutinib, osimertinib, brigatinib, dacomitinib, lapatinib, vandetanib, pyrotinib, mobocertinib, neratinib, and necitumumab) and EGFRi NOS. In some embodiments, the EGFR inhibitor is osimertinib. In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a MAPK-pathway inhibitor (e.g., BRAFi, panRAFi, MEKi, ERKi; PI3K-mTOR pathway inhibitors, such as alpha-specific PI3Ki, pan-class I PI3Ki and mTOR/PI3Ki, particularly everolimus and analogues thereof).

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an HDAC inhibitor or DNA methyltransferase inhibitor. In some embodiments, the HDAC inhibitor is Trichostatin A. In some embodiments, the DNA methyltransferase inhibitor is 5-azacytidine.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a MAT2A inhibitor (e.g., AG-270 (S 095033), IDE397, FIDAS-3, FIDAS-5, PF-9366, AGI-24512, AGI-25696, AGI-41998, AGI-43192, AGI-44131, ISM3412 (from InSilico Medicine), 28 (AZ series), 31 (AZ series), 32 (AZ series), 33 (AZ series), 001 (Simcere series), and 065 (Simcere series)).

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an inhibitor of a protein which interacts with or is required for PRMT5 function, including, but not limited to, pICIN, WDR77 or RIOK1.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an HDM2 inhibitor and/or 5-FU or other purine analogues (e.g., 6-thioguanine, 6-mercaptopurine).

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a CDK4 inhibitor, including, but not limited to, LEE011 or a CDK 4/6 inhibitor (e.g., palbociclib (Ibrance®), ribociclib (Kisqali®), and abemaciclib (Verzenio®).

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and targeted treatments contingent on the dependency of individual target tumors on relevant pathways as determined by suitable predictive markers, including but not limited to: inhibitors of HDM2i, PI3K/mTOR-I, MAPKi, RTKi (EGFRi, FGFRi, METi, IGFiRi, JAKi, and WNTi.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein) and immunotherapy.

In some embodiments, provided is a method of treating a disease or disorder (e.g., cancer) comprising administering or coadministering, in any order, to a patient in need thereof a cancer immunotherapy (e.g., a checkpoint blocking antibody) to treat a subject (e.g., a human subject), e.g., having a disease or disorder described herein (e.g., a cancer described herein)).

In some embodiments, the immunotherapeutic agent is an anti-CTLA-4 antibody (e.g., ipilimumab, tremelimumab).

In some embodiments, the immunotherapeutic agent is an anti-PD-1 ligand (e.g., PD-LI (e.g., B7-HI or CD274); or PD-L2 (e.g., B7-DC or CD273)). In some embodiments, the immunotherapeutic agent is an anti-PD-1 antibody (e.g., anti-PD-1 or anti-PD-L1, e.g., nivolumab (i.e., MDX-1106, BMS-936558, ONO-4538); CT-011; AMP-224; pembrolizumab; pidilizumab; or MK-3475). In some embodiments, the immunotherapeutic agent is an anti-PD-L1 antibody (e.g., BMS936559 (i.e., MDX-1105); MEDI4736; MSB0010718C (avelumab); or MPDL-3280A).

In some embodiments, the immunotherapeutic agent is a checkpoint blocking antibody (e.g., anti-TIM3, anti-LAG3, anti-TIGIT including IMP321 and MGA271).

In some embodiments, the immunotherapeutic agent is a cell-based therapy. In some embodiments, the cell-based therapy is a CAR-T therapy.

In some embodiments, the immunotherapeutic agent is a co-stimulatory antibody (e.g., anti-4-1BB, anti-OX40, anti-GITR, anti-CD27, anti-CD40).

In some embodiments, the immunotherapeutic agent is a cancer vaccine such as a neoantigen. These vaccines can be developed using peptides or RNA.

In some embodiments, the immunotherapeutic agent is an oncolytic virus.

In some embodiments, the immunotherapeutic agent is a STING pathway agonist. Exemplary STING agonists include MK-1454 and ADU-S100.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a disease-specific huMAB (e.g., an anti-HER3 huMAB.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an ADC/ADCC contingent on the expression of relevant surface targets on target tumors of interest.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and one or more DNA damage pathway inhibitor. In some embodiments, a DNA damage pathway inhibitor is selected from the group consisting of bleomycin, an ATM inhibitor (e.g., AZD1390), a USP1 inhibitor, a WEE1 inhibitor (e.g., AZD1775), and a Chk1 inhibitor (e.g., AZD7762). In some embodiments, a DNA damage pathway inhibitor is a DNA alkylating agent.

In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a PARP inhibitor. In some embodiments, a PARP inhibitor is selected from the group consisting of olaparib, rucaparib, niraparib, talazoparib, veliparib, pamiparib, CEP 9722, E7016, iniparib, and 3-aminobenzamide.

Some patients may experience allergic reactions to the PRMT5 inhibitors described herein and/or other anti-cancer agent(s) during or after administration; therefore, anti-allergic agents are often administered to minimize the risk of an allergic reaction. In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an anti-allergic agent (e.g., corticosteroids, including, but not limited to, dexamethasone (e.g., Decadron®), beclomethasone (e.g., Beclovent), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednesol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®)).

Some patients may experience nausea during and after administration of the PRMT5 inhibitors described herein and/or other anti-cancer agent(s); therefore, anti-emetics are used in preventing nausea (upper stomach) and vomiting. In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an anti-emetic (e.g., aprepitant (Emend®), ondansetron (Zofran®), granisetron HCl (Kytril®), lorazepam (Ativan®. dexamethasone (Decadron®), prochlorperazine (Compazine®), casopitant (Rezonic® and Zunrisa®), and combinations thereof).

Medication to alleviate the pain experienced during the treatment period is often prescribed to make the patient more comfortable. In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and an analgesic (e.g., an over-the-counter analgesic (e.g., Tylenol®), an opioid analgesic (e.g., hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®))).

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and a cytoprotective agent (e.g., Amifostine (Ethyon®, glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid)).

The structure of the active compounds identified by code numbers, generic or trade names may be taken from the actual edition of the standard compendium “The Merck Index” or from databases, e.g., Patents International (e.g., IMS World Publications).

The above-mentioned compounds, which can be used in combination with a PRMT5 inhibitor as described herein, can be prepared and administered as described in the art, including, but not limited to, in the documents cited above.

In one embodiment, provided are pharmaceutical compositions comprising at least one compound of the present disclosure (e.g., a crystalline form of a compound of Formula (I), e.g., crystalline form A) together with a pharmaceutically acceptable carrier suitable for administration to a human or animal subject, either alone or together with other anti-cancer agents.

In particular, compositions will either be formulated together as a combination therapeutic or administered separately.

In combination therapy, a PRMT5 inhibitor as described herein and other anti-cancer agent(s) may be administered either simultaneously, concurrently or sequentially with no specific time limits, wherein such administration provides therapeutically effective levels of the two compounds in the body of the patient.

In a preferred embodiment, the compound of the present disclosure (a compound of formula (I), crystalline forms (e.g., crystalline form A), pharmaceutical compositions (e.g., comprising a compound of formula (I) or a pharmaceutically acceptable salt thereof) or dosage forms thereof, as described herein) and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The PRMT5 inhibitor as described herein and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another aspect, provided are kits that include one or more PRMT5 inhibitor(s) as described herein (a compound of formula (I), crystalline forms (e.g., crystalline form A), pharmaceutical compositions (e.g., comprising a compound of formula (I) or a pharmaceutically acceptable salt thereof) or dosage forms thereof, as described herein) and a second therapeutic agent as disclosed herein are provided. Representative kits include (a) a PRMT5 inhibitor as described herein (a compound of formula (I), crystalline forms (e.g., crystalline form A), pharmaceutical compositions (e.g., comprising a compound of formula (I) or a pharmaceutically acceptable salt thereof) or dosage forms thereof, as described herein), (b) at least one other therapeutic agent, e.g., as indicated above, whereby such kit may comprise a package insert or other labeling including directions for administration.

A compound of formula (I), crystalline forms (e.g., crystalline form A), pharmaceutical compositions (e.g., comprising a compound of formula (I) or a pharmaceutically acceptable salt thereof) or dosage forms thereof, as described herein may also be used in combination with known therapeutic processes, for example, the administration of hormones or especially radiation. A compound of the present disclosure may in particular be used as a radiosensitizer, especially for the treatment of tumors which exhibit poor sensitivity to radiotherapy. In some embodiments, provided is a method of treating a disease or disorder (e.g., cancer) comprising administering or coadministering, in any order, to a patient in need thereof a compound of formula (I), crystalline forms (e.g., crystalline form A), pharmaceutical compositions (e.g., comprising a compound of formula (I) or a pharmaceutically acceptable salt thereof) or dosage forms thereof, as described herein, and radiation.

### Patient Selection and Monitoring

In some embodiments, provided is a method of determining if a subject having or having been diagnosed with a cancer (e.g., a cancer patient) will respond to therapeutic treatment with a PRMT5 inhibitor (e.g., an MTA-uncompetitive PRMT5 inhibitor, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein), comprising the steps of:
a) contacting a test sample obtained from said subject with a reagent capable of detecting human cancer cells that have MTAP deficiency and/or MTA accumulation; and
b) comparing the test sample with a reference (e.g., a reference sample taken from a non-cancerous or normal control subject),
wherein the presence of MTAP deficiency and/or MTA accumulation in said test sample indicates that the subject will respond to therapeutic treatment with a PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein).

In some embodiments, provided is a method of determining if a cancer will respond to therapeutic treatment with a PRMT5 inhibitor (e.g., an MTA-uncompetitive, non-competitive, or mixed mode PRMT5 inhibitor or an MTA-cooperative binding agent, a compound of formula (I), crystalline forms (e.g., crystalline form A), pharmaceutical compositions (e.g., comprising a compound of formula (I) or a pharmaceutically acceptable salt thereof) or dosage forms thereof, as described herein), comprising the steps of:
a) contacting a test sample obtained from a subject having or having been diagnosed with said cancer with a reagent capable of detecting human cancer cells that have MTAP deficiency and/or MTA accumulation; and
b) comparing the test sample with a reference (e.g., a reference sample taken from a non-cancerous or normal control subject), wherein the presence of MTAP deficiency and/or MTA accumulation in said test sample indicates that the cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein). In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma, e.g., pancreatic ductal adenocarcinoma (PDAC)), mesothelioma, melanoma, non-small cell lung cancer (NSCLC; e.g., lung squamous or lung adenocarcinoma), astrocytoma, undifferentiated pleomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the cancer is a CNS cancer. In some embodiments, the cancer is not a CNS cancer.

In some embodiments, the cancer is a cancer selected from the group of glioblastoma, glioblastoma multiforme, urothelial carcinoma, malignant peripheral nerve sheath tumor (MPNST), cholangiocarcinoma, NSCLC (e.g., squamous and adenocarcinoma), bladder cancer, pancreatic cancer (e.g., pancreatic ductal adenocarcinoma (PDAC)), mesothelioma, colorectal cancer, chronic myelogenous leukemia (CML), and diffuse large B-cell lymphoma (DLBCL).

In some embodiments, the cancer is a cancer selected from the group of glioblastoma, glioblastoma multiforme, cholangiocarcinoma, NSCLC (e.g., squamous and adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group consisting of glioma, glioblastoma, non-small cell lung cancer (adenocarcinoma and squamous), mesothelioma, cholangiocarcinoma, bladder cancer, urothelial carcinoma, pancreatic cancer (e.g., pancreatic ductal adenocarcinoma (PDAC)) and malignant peripheral nerve sheath tumor.

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) and mesothelioma.

In some embodiments, the cancer is a cancer selected from the group of cholangiocarcinoma, NSCLC (squamous), bladder cancer, pancreatic cancer (e.g., pancreatic ductal adenocarcinoma (PDAC)) and mesothelioma.

In some embodiments, the cancer is glioma. In some embodiments, the cancer is glioblastoma. In some embodiments, the cancer is glioblastoma multiforme. In some embodiments, the cancer is urothelial carcinoma, malignant peripheral nerve sheath tumor (MPNST). In some embodiments, the cancer is cholangiocarcinoma. In some embodiments, the cancer is NSCLC (e.g., lung squamous and lung adenocarcinoma). In some embodiments, the cancer is squamous NSCLC. In some embodiments, the cancer is NSCLC adenocarcinoma. In some embodiments, the cancer is bladder cancer (e.g., bladder urothelial carcinoma). In some embodiments, the cancer is pancreatic cancer (e.g., pancreatic ductal adenocarcinoma (PDAC)). In some embodiments, the cancer is pancreatic ductal adenocarcinoma (PDAC). In some embodiments, the cancer is mesothelioma. In some embodiments, the cancer is colorectal cancer. In some embodiments, the cancer is chronic myelogenous leukemia (CML). In some embodiments, the cancer is diffuse large B-cell lymphoma (DLBCL). In some embodiments, the cancer is malignant peripheral nerve sheath tumors (MPNST). In some embodiments, the cancer is esophageal cancer (e.g., esophageal squamous cell carcinoma or esophageal adenocarcinoma). In some embodiments, the cancer is melanoma. In some embodiments, the cancer is astrocytoma. In some embodiments, the cancer is undifferentiated pleomorphic sarcoma. In some embodiments, the cancer is leukemia. In some embodiments, the cancer is head and neck cancer. In some embodiments, the cancer is stomach adenocarcinoma. In some embodiments, the cancer is myxofibrosarcoma. In some embodiments, the cancer is cholangiosarcoma. In some embodiments, the cancer is cancer of the brain. In some embodiments, the cancer is stomach cancer. In some embodiments, the cancer is kidney cancer. In some embodiments, the cancer is breast cancer. In some embodiments, the cancer is endometrial cancer. In some embodiments, the cancer is urinary tract cancer. In some embodiments, the cancer is liver cancer. In some embodiments, the cancer is soft tissue cancer. In some embodiments, the cancer is pleura cancer. In some embodiments, the cancer is large intestine cancer. In some embodiments, the cancer is sarcoma.

In some embodiments, the method further comprises the step of determining the level of PRMT5 in the cancer cells. The level of expression of PRMT5 can be considered when determining the therapeutically effective dosage of a PRMT5 inhibitor.

In one aspect, provided is a method of determining the sensitivity of a cancer cell to PRMT5 inhibition (e.g., inhibition with an MTA-uncompetitive PRMT5 inhibitor, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein), comprising the steps of:
a) assaying the production, level, activity, expression or presence of MTAP), in said cancer cell;
b) comparing the production, level, activity, expression or presence of MTAP in the cancer cell with the production, level, activity, expression or presence of MTAP, respectively, in a non-cancerous or normal control cell,
   wherein a decreased level, activity or expression in the cancer cell indicates MTAP deficiency and wherein MTAP deficiency indicates that said cancer cell is sensitive to the PRMT5 inhibitor.

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma, e.g., pancreatic ductal adenocarcinoma (PDAC)), mesothelioma, melanoma, non-small cell lung cancer (NSCLC; e.g., lung squamous or lung adenocarcinoma), astrocytoma, undifferentiated pleomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the cancer is a CNS cancer. In some embodiments, the cancer is not a CNS cancer.

In some embodiments, the cancer is a cancer selected from the group of glioblastoma, glioblastoma multiforme, urothelial carcinoma, malignant peripheral nerve sheath tumor (MPNST), cholangiocarcinoma, NSCLC (e.g., squamous and adenocarcinoma), bladder cancer, pancreatic cancer (e.g., pancreatic ductal adenocarcinoma (PDAC)), mesothelioma, colorectal cancer, chronic myelogenous leukemia (CML), and diffuse large B-cell lymphoma (DLBCL).

In some embodiments, the cancer is a cancer selected from the group of glioblastoma, glioblastoma multiforme, cholangiocarcinoma, NSCLC (e.g., squamous and adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group consisting of glioma, glioblastoma, non-small cell lung cancer (adenocarcinoma and squamous), mesothelioma, cholangiocarcinoma, bladder cancer, urothelial carcinoma, pancreatic cancer (e.g., pancreatic ductal adenocarcinoma (PDAC)) and malignant peripheral nerve sheath tumor.

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) and mesothelioma.

In some embodiments, the cancer is a cancer selected from the group of cholangiocarcinoma, NSCLC (squamous), bladder cancer, pancreatic cancer (e.g., pancreatic ductal adenocarcinoma (PDAC)) and mesothelioma.

In some embodiments, the cancer is glioma. In some embodiments, the cancer is glioblastoma. In some embodiments, the cancer is glioblastoma multiforme. In some embodiments, the cancer is urothelial carcinoma, malignant peripheral nerve sheath tumor (MPNST). In some embodiments, the cancer is cholangiocarcinoma. In some embodiments, the cancer is NSCLC (e.g., lung squamous and lung adenocarcinoma). In some embodiments, the cancer is squamous NSCLC. In some embodiments, the cancer is NSCLC adenocarcinoma. In some embodiments, the cancer is bladder cancer (e.g., bladder urothelial carcinoma). In some embodiments, the cancer is pancreatic cancer (e.g., pancreatic ductal adenocarcinoma (PDAC)). In some embodiments, the cancer is pancreatic ductal adenocarcinoma (PDAC). In some embodiments, the cancer is mesothelioma. In some embodiments, the cancer is colorectal cancer. In some embodiments, the cancer is chronic myelogenous leukemia (CML). In some embodiments, the cancer is diffuse large B-cell lymphoma (DLBCL). In some embodiments, the cancer is malignant peripheral nerve sheath tumors (MPNST). In some embodiments, the cancer is esophageal cancer (e.g., esophageal squamous cell carcinoma or esophageal adenocarcinoma). In some embodiments, the cancer is melanoma. In some embodiments, the cancer is astrocytoma. In some embodiments, the cancer is undifferentiated pleomorphic sarcoma. In some embodiments, the cancer is leukemia. In some embodiments, the cancer is head and neck cancer. In some embodiments, the cancer is stomach adenocarcinoma. In some embodiments, the cancer is myxofibrosarcoma. In some embodiments, the cancer is cholangiosarcoma. In some embodiments, the cancer is cancer of the brain. In some embodiments, the cancer is stomach cancer. In some embodiments, the cancer is kidney cancer. In some embodiments, the cancer is breast cancer. In some embodiments, the cancer is endometrial cancer. In some embodiments, the cancer is urinary tract cancer. In some embodiments, the cancer is liver cancer. In some embodiments, the cancer is soft tissue cancer. In some embodiments, the cancer is pleura cancer. In some embodiments, the cancer is large intestine cancer. In some embodiments, the cancer is sarcoma.

In one embodiment, provided is a method of determining the sensitivity of a cancer cell to a PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein), comprising the steps of:
a) assaying for level, activity or expression of the MTAP gene or its gene product in both the cancer cell and a normal control cell, wherein a decreased level, activity or expression in the cancer cell indicates MTAP deficiency; b) assaying for PRMT5 expression in said cancer cell; c) comparing the PRMT5 expression with PRMT5 expression in the cancer cell and a normal control cell; wherein the similarity in PRMT5 expression, and the presence of said MTAP deficiency in said cancer cell, indicates said cell is sensitive to a PRMT5 inhibitor.

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma, e.g., pancreatic ductal adenocarcinoma (PDAC)), mesothelioma, melanoma, non-small cell lung cancer (NSCLC; e.g., lung squamous or lung adenocarcinoma), astrocytoma, undifferentiated pleomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the cancer is a CNS cancer. In some embodiments, the cancer is not a CNS cancer.

In some embodiments, the cancer is a cancer selected from the group of glioblastoma, glioblastoma multiforme, urothelial carcinoma, malignant peripheral nerve sheath tumor (MPNST), cholangiocarcinoma, NSCLC (e.g., squamous and adenocarcinoma), bladder cancer, pancreatic cancer (e.g., pancreatic ductal adenocarcinoma (PDAC)), mesothelioma, colorectal cancer, chronic myelogenous leukemia (CML), and diffuse large B-cell lymphoma (DLBCL).

In some embodiments, the cancer is a cancer selected from the group of glioblastoma, glioblastoma multiforme, cholangiocarcinoma, NSCLC (e.g., squamous and adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group consisting of glioma, glioblastoma, non-small cell lung cancer (adenocarcinoma and squamous), mesothelioma, cholangiocarcinoma, bladder cancer, urothelial carcinoma, pancreatic cancer (e.g., pancreatic ductal adenocarcinoma (PDAC)) and malignant peripheral nerve sheath tumor.

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) and mesothelioma.

In some embodiments, the cancer is a cancer selected from the group of cholangiocarcinoma, NSCLC (squamous), bladder cancer, pancreatic cancer (e.g., pancreatic ductal adenocarcinoma (PDAC)) and mesothelioma.

In some embodiments, the cancer is glioma. In some embodiments, the cancer is glioblastoma. In some embodiments, the cancer is glioblastoma multiforme. In some embodiments, the cancer is urothelial carcinoma, malignant peripheral nerve sheath tumor (MPNST). In some embodiments, the cancer is cholangiocarcinoma. In some embodiments, the cancer is NSCLC (e.g., lung squamous and lung adenocarcinoma). In some embodiments, the cancer is squamous NSCLC. In some embodiments, the cancer is NSCLC adenocarcinoma. In some embodiments, the cancer is bladder cancer (e.g., bladder urothelial carcinoma). In some embodiments, the cancer is pancreatic cancer (e.g., pancreatic ductal adenocarcinoma (PDAC)). In some embodiments, the cancer is pancreatic ductal adenocarcinoma (PDAC). In some embodiments, the cancer is mesothelioma. In some embodiments, the cancer is colorectal cancer. In some embodiments, the cancer is chronic myelogenous leukemia (CML). In some embodiments, the cancer is diffuse large B-cell lymphoma (DLBCL). In some embodiments, the cancer is malignant peripheral nerve sheath tumors (MPNST). In some embodiments, the cancer is esophageal cancer (e.g., esophageal squamous cell carcinoma or esophageal adenocarcinoma). In some embodiments, the cancer is melanoma. In some embodiments, the cancer is astrocytoma. In some embodiments, the cancer is undifferentiated pleomorphic sarcoma. In some embodiments, the cancer is leukemia. In some embodiments, the cancer is head and neck cancer. In some embodiments, the cancer is stomach adenocarcinoma. In some embodiments, the cancer is myxofibrosarcoma. In some embodiments, the cancer is cholangiosarcoma. In some embodiments, the cancer is cancer of the brain. In some embodiments, the cancer is stomach cancer. In some embodiments, the cancer is kidney cancer. In some embodiments, the cancer is breast cancer. In some embodiments, the cancer is endometrial cancer. In some embodiments, the cancer is urinary tract cancer. In some embodiments, the cancer is liver cancer. In some embodiments, the cancer is soft tissue cancer. In some embodiments, the cancer is pleura cancer. In some embodiments, the cancer is large intestine cancer. In some embodiments, the cancer is sarcoma.

In one aspect the provided is a therapeutic method of treating a subject having or having been diagnosed with a cancer (e.g., a cancer associated with MTAP deficiency and/or MTA accumulation) comprising the steps of:
a) assessing the level of MTAP and/or MTA in a test sample obtained from said subject (e.g., by contacting the sample with a reagent capable of detecting human MTAP-deficient and/or MTA-accumulating cancer cells in a test sample obtained from said subject), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said subject will respond to therapeutic treatment with a PRMT5 inhibitor; and
c) administering a therapeutically effective amount of PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) to the subject identified in step b).

In one aspect provided is a therapeutic method of treating a cancer (e.g., a cancer associated with MTAP deficiency and/or MTA accumulation) in a subject in need thereof comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) to the subject identified in step b).

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma, e.g., pancreatic ductal adenocarcinoma (PDAC)), mesothelioma, melanoma, non-small cell lung cancer (NSCLC; e.g., lung squamous or lung adenocarcinoma), astrocytoma, undifferentiated pleomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the cancer is a CNS cancer. In some embodiments, the cancer is not a CNS cancer.

In some embodiments, the cancer is a cancer selected from the group of glioblastoma, glioblastoma multiforme, urothelial carcinoma, malignant peripheral nerve sheath tumor (MPNST), cholangiocarcinoma, NSCLC (e.g., squamous and adenocarcinoma), bladder cancer, pancreatic cancer (e.g., pancreatic ductal adenocarcinoma (PDAC)), mesothelioma, colorectal cancer, chronic myelogenous leukemia (CML), and diffuse large B-cell lymphoma (DLBCL).

In some embodiments, the cancer is a cancer selected from the group of glioblastoma, glioblastoma multiforme, cholangiocarcinoma, NSCLC (e.g., squamous and adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group consisting of glioma, glioblastoma, non-small cell lung cancer (adenocarcinoma and squamous), mesothelioma, cholangiocarcinoma, bladder cancer, urothelial carcinoma, pancreatic cancer (e.g., pancreatic ductal adenocarcinoma (PDAC)) and malignant peripheral nerve sheath tumor.

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) and mesothelioma.

In some embodiments, the cancer is a cancer selected from the group of cholangiocarcinoma, NSCLC (squamous), bladder cancer, pancreatic cancer (e.g., pancreatic ductal adenocarcinoma (PDAC)) and mesothelioma.

In some embodiments, the cancer is glioma. In some embodiments, the cancer is glioblastoma. In some embodiments, the cancer is glioblastoma multiforme. In some embodiments, the cancer is urothelial carcinoma, malignant peripheral nerve sheath tumor (MPNST). In some embodiments, the cancer is cholangiocarcinoma. In some embodiments, the cancer is NSCLC (e.g., lung squamous and lung adenocarcinoma). In some embodiments, the cancer is squamous NSCLC. In some embodiments, the cancer is NSCLC adenocarcinoma. In some embodiments, the cancer is bladder cancer (e.g., bladder urothelial carcinoma). In some embodiments, the cancer is pancreatic cancer (e.g., pancreatic ductal adenocarcinoma (PDAC)). In some embodiments, the cancer is pancreatic ductal adenocarcinoma (PDAC). In some embodiments, the cancer is mesothelioma. In some embodiments, the cancer is colorectal cancer. In some embodiments, the cancer is chronic myelogenous leukemia (CML). In some embodiments, the cancer is diffuse large B-cell lymphoma (DLBCL). In some embodiments, the cancer is malignant peripheral nerve sheath tumors (MPNST). In some embodiments, the cancer is esophageal cancer (e.g., esophageal squamous cell carcinoma or esophageal adenocarcinoma). In some embodiments, the cancer is melanoma. In some embodiments, the cancer is astrocytoma. In some embodiments, the cancer is undifferentiated pleomorphic sarcoma. In some embodiments, the cancer is leukemia. In some embodiments, the cancer is head and neck cancer. In some embodiments, the cancer is stomach adenocarcinoma. In some embodiments, the cancer is myxofibrosarcoma. In some embodiments, the cancer is cholangiosarcoma. In some embodiments, the cancer is cancer of the brain. In some embodiments, the cancer is stomach cancer. In some embodiments, the cancer is kidney cancer. In some embodiments, the cancer is breast cancer. In some embodiments, the cancer is endometrial cancer. In some embodiments, the cancer is urinary tract cancer. In some embodiments, the cancer is liver cancer. In some embodiments, the cancer is soft tissue cancer. In some embodiments, the cancer is pleura cancer. In some embodiments, the cancer is large intestine cancer. In some embodiments, the cancer is sarcoma.

In some embodiments, the method further comprises the step of determining the level of PRMT5 in the cancer cells.

In one aspect provided is a therapeutic method of treating a subject having or having been diagnosed with a cancer associated with MTAP deficiency and/or MTA accumulation comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference sample (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of a composition comprising a PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) to the subject identified in step b).

In one aspect provided is a therapeutic method of treating cancer associated with MTAP deficiency and/or MTA accumulation in a subject in need thereof comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference sample (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of a composition comprising a PRMT5 inhibitor (e.g., an MTA-uncompetitive PRMT5 inhibitor e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) to the subject identified in step b).

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma, e.g., pancreatic ductal adenocarcinoma (PDAC)), mesothelioma, melanoma, non-small cell lung cancer (NSCLC; e.g., lung squamous or lung adenocarcinoma), astrocytoma, undifferentiated pleomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the cancer is a CNS cancer. In some embodiments, the cancer is not a CNS cancer.

In some embodiments, the cancer is a cancer selected from the group of glioblastoma, glioblastoma multiforme, urothelial carcinoma, malignant peripheral nerve sheath tumor (MPNST), cholangiocarcinoma, NSCLC (e.g., squamous and adenocarcinoma), bladder cancer, pancreatic cancer (e.g., pancreatic ductal adenocarcinoma (PDAC)), mesothelioma, colorectal cancer, chronic myelogenous leukemia (CML), and diffuse large B-cell lymphoma (DLBCL).

In some embodiments, the cancer is a cancer selected from the group of glioblastoma, glioblastoma multiforme, cholangiocarcinoma, NSCLC (e.g., squamous and adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group consisting of glioma, glioblastoma, non-small cell lung cancer (adenocarcinoma and squamous), mesothelioma, cholangiocarcinoma, bladder cancer, urothelial carcinoma, pancreatic cancer (e.g., pancreatic ductal adenocarcinoma (PDAC)) and malignant peripheral nerve sheath tumor.

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) and mesothelioma.

In some embodiments, the cancer is a cancer selected from the group of cholangiocarcinoma, NSCLC (squamous), bladder cancer, pancreatic cancer (e.g., pancreatic ductal adenocarcinoma (PDAC)) and mesothelioma.

In some embodiments, the cancer is glioma. In some embodiments, the cancer is glioblastoma. In some embodiments, the cancer is glioblastoma multiforme. In some embodiments, the cancer is urothelial carcinoma, malignant peripheral nerve sheath tumor (MPNST). In some embodiments, the cancer is cholangiocarcinoma. In some embodiments, the cancer is NSCLC (e.g., lung squamous and lung adenocarcinoma). In some embodiments, the cancer is squamous NSCLC. In some embodiments, the cancer is NSCLC adenocarcinoma. In some embodiments, the cancer is bladder cancer (e.g., bladder urothelial carcinoma). In some embodiments, the cancer is pancreatic cancer (e.g., pancreatic ductal adenocarcinoma (PDAC)). In some embodiments, the cancer is pancreatic ductal adenocarcinoma (PDAC). In some embodiments, the cancer is mesothelioma. In some embodiments, the cancer is colorectal cancer. In some embodiments, the cancer is chronic myelogenous leukemia (CML). In some embodiments, the cancer is diffuse large B-cell lymphoma (DLBCL). In some embodiments, the cancer is malignant peripheral nerve sheath tumors (MPNST). In some embodiments, the cancer is esophageal cancer (e.g., esophageal squamous cell carcinoma or esophageal adenocarcinoma). In some embodiments, the cancer is melanoma. In some embodiments, the cancer is astrocytoma. In some embodiments, the cancer is undifferentiated pleomorphic sarcoma. In some embodiments, the cancer is leukemia. In some embodiments, the cancer is head and neck cancer. In some embodiments, the cancer is stomach adenocarcinoma. In some embodiments, the cancer is myxofibrosarcoma. In some embodiments, the cancer is cholangiosarcoma. In some embodiments, the cancer is cancer of the brain. In some embodiments, the cancer is stomach cancer. In some embodiments, the cancer is kidney cancer. In some embodiments, the cancer is breast cancer. In some embodiments, the cancer is endometrial cancer. In some embodiments, the cancer is urinary tract cancer. In some embodiments, the cancer is liver cancer. In some embodiments, the cancer is soft tissue cancer. In some embodiments, the cancer is pleura cancer. In some embodiments, the cancer is large intestine cancer. In some embodiments, the cancer is sarcoma.

In some embodiments, the method further comprises the step of determining the level of PRMT5 in the cancer cells.

In some embodiments provided is a method of determining if a subject having or having been diagnosed with a cancer associated with MTAP deficiency and/or MTA accumulation will respond to treatment with a PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said subject will respond to therapeutic treatment with a PRMT5 inhibitor (e.g., an MTA-uncompetitive, non-competitive, or mixed mode PRMT5 inhibitor or an MTA-cooperative binding agent).

In some embodiments provided is a method of determining if a cancer associated with MTAP deficiency and/or MTA accumulation will respond to treatment with a PRMT5 inhibitor (e.g., an MTA-uncompetitive PRMT5 inhibitor, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) comprising the steps of:
a) assessing the level of MTAP and/or MTA in a test sample obtained from a subject having or having been diagnosed with said cancer (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent).

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma, e.g., pancreatic ductal adenocarcinoma (PDAC)), mesothelioma, melanoma, non-small cell lung cancer (NSCLC; e.g., lung squamous or lung adenocarcinoma), astrocytoma, undifferentiated pleomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the cancer is a CNS cancer. In some embodiments, the cancer is not a CNS cancer.

In some embodiments, the cancer is a cancer selected from the group of glioblastoma, glioblastoma multiforme, urothelial carcinoma, malignant peripheral nerve sheath tumor (MPNST), cholangiocarcinoma, NSCLC (e.g., squamous and adenocarcinoma), bladder cancer, pancreatic cancer (e.g., pancreatic ductal adenocarcinoma (PDAC)), mesothelioma, colorectal cancer, chronic myelogenous leukemia (CML), and diffuse large B-cell lymphoma (DLBCL).

In some embodiments, the cancer is a cancer selected from the group of glioblastoma, glioblastoma multiforme, cholangiocarcinoma, NSCLC (e.g., squamous and adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) mesothelioma, colorectal cancer, chronic myelogenous leukemia (CML), and diffuse large B-cell lymphoma (DLBCL).

In some embodiments, the cancer is a cancer selected from the group consisting of glioma, glioblastoma, non-small cell lung cancer (adenocarcinoma and squamous), mesothelioma, cholangiocarcinoma, bladder cancer, urothelial carcinoma, pancreatic cancer (e.g., pancreatic ductal adenocarcinoma (PDAC)) and malignant peripheral nerve sheath tumor.

In some embodiments, the cancer is a cancer selected from the group of cholangiocarcinoma, NSCLC (squamous), NSCLC (adenocarcinoma), bladder cancer, pancreatic cancer (e.g., pancreatic ductal adenocarcinoma (PDAC)) and mesothelioma.

In some embodiments, the cancer is a cancer selected from the group of cholangiocarcinoma, NSCLC (squamous), bladder cancer, pancreatic cancer (e.g., pancreatic ductal adenocarcinoma (PDAC)) and mesothelioma.

In some embodiments, the cancer is glioma. In some embodiments, the cancer is glioblastoma. In some embodiments, the cancer is glioblastoma multiforme. In some embodiments, the cancer is urothelial carcinoma, malignant peripheral nerve sheath tumor (MPNST). In some embodiments, the cancer is cholangiocarcinoma. In some embodiments, the cancer is NSCLC (e.g., lung squamous and lung adenocarcinoma). In some embodiments, the cancer is squamous NSCLC. In some embodiments, the cancer is NSCLC adenocarcinoma. In some embodiments, the cancer is bladder cancer (e.g., bladder urothelial carcinoma). In some embodiments, the cancer is pancreatic cancer (e.g., pancreatic ductal adenocarcinoma (PDAC)). In some embodiments, the cancer is pancreatic ductal adenocarcinoma (PDAC). In some embodiments, the cancer is mesothelioma. In some embodiments, the cancer is colorectal cancer. In some embodiments, the cancer is chronic myelogenous leukemia (CML). In some embodiments, the cancer is diffuse large B-cell lymphoma (DLBCL). In some embodiments, the cancer is malignant peripheral nerve sheath tumors (MPNST). In some embodiments, the cancer is esophageal cancer (e.g., esophageal squamous cell carcinoma or esophageal adenocarcinoma). In some embodiments, the cancer is melanoma. In some embodiments, the cancer is astrocytoma. In some embodiments, the cancer is undifferentiated pleomorphic sarcoma. In some embodiments, the cancer is leukemia. In some embodiments, the cancer is head and neck cancer. In some embodiments, the cancer is stomach adenocarcinoma. In some embodiments, the cancer is myxofibrosarcoma. In some embodiments, the cancer is cholangiosarcoma. In some embodiments, the cancer is cancer of the brain. In some embodiments, the cancer is stomach cancer. In some embodiments, the cancer is kidney cancer. In some embodiments, the cancer is breast cancer. In some embodiments, the cancer is endometrial cancer. In some embodiments, the cancer is urinary tract cancer. In some embodiments, the cancer is liver cancer. In some embodiments, the cancer is soft tissue cancer. In some embodiments, the cancer is pleura cancer. In some embodiments, the cancer is large intestine cancer. In some embodiments, the cancer is sarcoma.

In some embodiments, the method further comprises the step of determining the level of PRMT5 in the cancer cells.

### Sample Preparation

Further provided are assays for the detection of MTAP deficiency and/or MTA accumulation. They can include detecting a mutation related to MTAP deficiency and/or MTA accumulation, e.g., in a body fluid such as blood (e.g., serum or plasma) bone marrow, cerebral spinal fluid, peritoneal/pleural fluid, lymph fluid, ascites, serous fluid, sputum, lacrimal fluid, stool, and urine, or in a tissue such as a tumor tissue. The tumor tissue can be fresh tissue or preserved tissue (e.g., formalin fixed tissue, e.g., paraffin-embedded tissue).

Body fluid samples can be obtained from a subject using any of the methods known in the art. Methods for extracting cellular DNA from body fluid samples are well known in the art. Typically, cells are lysed with detergents. After cell lysis, proteins are removed from DNA using various proteases. DNA is then extracted with phenol, precipitated in alcohol, and dissolved in an aqueous solution. Methods for extracting acellular DNA from body fluid samples are also known in the art. Commonly, a cellular DNA in a body fluid sample is separated from cells, precipitated in alcohol, and dissolved in an aqueous solution.

### Detection of PRMT5 Selectivity

Samples, once prepared, can be tested for MTAP deficiency and/or MTA accumulation, either or both of which indicates that the sample is sensitive to treatment with a PRMT5 inhibitor. Cells can be determined to be MTA accumulating by techniques known in the art; methods for detecting MTA include, as a non-limiting example, liquid chromatography—electrospray ionization—tandem mass spectrometry (LC-ESI-MS/MS), as described in Stevens et al. 2010. J. Chromatogr. A. 1217: 3282-3288; and Kirovski et al. 2011 Am. J. Pathol. 178: 1145-1152; and references cited therein. The detection of MTAP deficiency can be done by any number of ways, for example: DNA sequencing, PCR based methods, including RT-PCR, microarray analysis, Southern blotting, Northern blotting, Next Generation Sequencing, and dip stick analysis. In some embodiments, MTAP deficiency is evaluated by any technique known in the art, for example, immunohistochemistry utilizing an anti-MTAP antibody or derivative thereof, and/or genomic sequencing, or nucleic acid hybridization, or amplification utilizing at least one probe or primer comprising a sequence of at least 12 contiguous nucleotides (nt) of the sequence of MTAP wherein the primer is no longer than about 30 nt.

The polymerase chain reaction (PCR) can be used to amplify and identify MTAP deficiency from either genomic DNA or RNA extracted from tumor tissue. PCR is well known in the art and is described in detail in Saiki et al., Science 1988, 239:487.

Methods of detecting MTAP deficiency by hybridization are provided. The method comprises identifying MTAP deficiency in a sample by its inability to hybridize to MTAP nucleic acid. The nucleic acid probe is detectably labeled with a label such as a radioisotope, a fluorescent agent or a chromogenic agent. Radioisotopes can include without limitation; 3H, 32P, 33P and 35S etc. Fluorescent agents can include without limitation: FITC, texas red, rhodamine, etc.

The probe used in detection that is capable of hybridizing to MTAP nucleic acid can be from about 8 nucleotides to about 100 nucleotides, from about 10 nucleotides to about 75 nucleotides, from about 15 nucleotides to about 50 nucleotides, or about 20 to about 30 nucleotides. The kit can also provide instructions for analysis of patient cancer samples, wherein the presence or absence of MTAP deficiency indicates if the subject is sensitive or insensitive to treatment with a PRMT5 inhibitor.

Single stranded conformational polymorphism (SSCP) can also be used to detect MTAP deficiency. This technique is well described in Orita et al., PNAS 1989, 86:2766-2770.

### Measurement of Gene Expression

Evaluation of MTAP deficiency and measurement of MTAP gene expression, and measurement of PRMT5 gene expression can be performed using any method or reagent known in the art.

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix™ U133 microarray chips.

In one aspect, gene expression is detected and quantitated by hybridization to a probe that specifically hybridizes to the appropriate probe for that biomarker. The probes also can be attached to a solid support for use in high throughput screening assays using methods known in the art.

In one aspect, the expression level of a gene is determined through exposure of a nucleic acid sample to the probe-modified chip. Extracted nucleic acid is labeled, for example, with a fluorescent tag, preferably during an amplification step.

Hybridization of the labeled sample is performed at an appropriate stringency level. The degree of probe-nucleic acid hybridization is quantitatively measured using a detection device.

Alternatively, any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination. In one embodiment, the hybridized nucleic acids are detected by detecting one or more labels attached to the sample nucleic acids. The labels can be incorporated by any of a number of means well known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g., fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

In one example, the gene expression can be measured through an in-situ hybridization protocol that can detect RNA molecules on a slide containing tissue sections or cells (e.g., through RNAscope®)).

Detectable labels suitable for use in the methods disclosed herein include any composition detectable by spectroscopic, photochemical, biochemical, immunochemical, electrical, optical or chemical means. Useful labels include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 125I, 35S, 14C, or 32P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

### Detection of Polypeptides

Protein levels of MTAP can be determined by examining protein expression or the protein product. Determining the protein level involves measuring the amount of any immunospecific binding that occurs between an antibody that selectively recognizes and binds to the polypeptide of the biomarker in a sample obtained from a subject and comparing this to the amount of immunospecific binding of at least one biomarker in a control sample.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), Western blot analysis, immunoprecipitation assays, immunofluorescent assays, flow cytometry, immunohistochemistry, HPLC, mass spectrometry, confocal microscopy, enzymatic assays, surface plasmon resonance and PAGE-SDS.

### Adjacent Biomarkers

Near or adjacent to MTAP on chromosome 9 are several other biomarkers. CDKN2A is often, if not usually, deleted along with MTAP. Additional genes or pseudogenes in this region include: C9orf53, ERVFRD-3, TUBB8P1, KHSRPP1, MIR31, and MIR31HG.

In some embodiments of the methods, the cell that is MTAP-deficient is also deficient in CDKN2A. In some embodiments, the cell that is MTAP-deficient is also deficient in one or more of: CDKN2A, C9orf53, ERVFRD-3, TUBB8P1, KHSRPP1, MIR31, and MIR31HG.

Thus, in various methods involving a step of evaluating a cell for MTAP deficiency or determining if a cell is MTAP-deficient, this step can comprise the step of determining if the cell is deficient for one or more of these markers: CDKN2A, C9orf53, ERVFRD-3, TUBB8P1, KHSRPP1, MIR31, and MIR31HG.

Thus, in some embodiments, the disclosure encompasses: A method of determining if a subject having or having been diagnosed with a cancer will respond to therapeutic treatment with a PRMT5 inhibitor (e.g., an MTA-uncompetitive, non-competitive, or mixed mode PRMT5 inhibitor or an MTA-cooperative binding agent), comprising the steps of:
a) evaluating a test sample obtained from said subject for MTAP deficiency, and evaluating a reference sample from a non-cancerous or normal control subject for MTAP deficiency, wherein MTAP deficiency in the test sample relative to the reference sample indicates that the subject will respond to therapeutic treatment with a PRMT5 inhibitor (e.g., an MTA-uncompetitive PRMT5 inhibitor, e.g., a crystalline form of a compound of Formula (I)); wherein MTAP deficiency is evaluated by evaluating the deficiency of one or more of the following biomarkers: CDKN2A, C9orf53, ERVFRD-3, TUBB8P1, KHSRPP1, MIR31, and MIR31HG, and wherein the method can further comprise the following steps:
b) determining the level of MTAP in the subject, wherein steps a) and b) can be performed in any order;
c) administering a therapeutically effective amount of a PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) to the subject; and
d) determining the level of PRMT5 activity in the subject following step c), wherein a decrease in the level of PRMT5 activity is correlated with the inhibition of the proliferation of the cancer, and wherein steps c) and d) are performed after steps a) and b).

In some embodiments, the disclosure encompasses: A method of determining if a cancer will respond to therapeutic treatment with a PRMT5 inhibitor (e.g., an MTA-uncompetitive, non-competitive, or mixed mode PRMT5 inhibitor or an MTA-cooperative binding agent), comprising the steps of:
a) evaluating a test sample obtained from a subject having or having been diagnosed with said cancer for MTAP deficiency, and evaluating a reference sample from a non-cancerous or normal control subject for MTAP deficiency, wherein MTAP deficiency in the test sample relative to the reference sample indicates that the cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein); wherein MTAP deficiency is evaluated by evaluating the deficiency of one or more of the following biomarkers: CDKN2A, C9orf53, ERVFRD-3, TUBB8P1, KHSRPP1, MIR31, and MIR31HG, and wherein the method can further comprise the following steps:
b) determining the level of MTAP in the subject, wherein steps a) and b) can be performed in any order;
c) administering a therapeutically effective amount of a PRMT5 inhibitor (e.g., an MTA-uncompetitive PRMT5 inhibitor, e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein) to the subject; and
d) determining the level of PRMT5 activity in the subject following step c), wherein a decrease in the level of PRMT5 activity is correlated with the inhibition of the proliferation of the cancer, and wherein steps c) and d) are performed after steps a) and b).

### Assaying for Biomarkers and PRMT5 Inhibitor Treatment

A number of patient stratification strategies could be employed to find patients likely to be sensitive to PRMT5 inhibition with an MTA-uncompetitive, non-competitive, or mixed mode PRMT5 inhibitor or an MTA-cooperative binding agent (e.g., a compound of formula (I), crystalline forms (e.g., crystalline form A), pharmaceutical compositions (e.g., comprising a compound of formula (I) or a pharmaceutically acceptable salt thereof) or dosage forms thereof, as described herein), including but not limited to, testing for MTAP deficiency and/or MTA accumulation.

Once a patient has been assayed for MTAP deficiency and/or MTA accumulation and predicted to be sensitive to treatment with a PRMT5 inhibitor, administration of any PRMT5 inhibitor (e.g., an MTA-uncompetitive, non-competitive, or mixed mode PRMT5 inhibitor or an MTA-cooperative binding agent, e.g., a crystalline form of a compound of Formula (I)) to a patient can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents may be empirically adjusted.

### Dosage

The dose ranges and recitations below refer to the dose of the compound of formula (I) contained in the dosage forms and pharmaceutical compositions described herein that are administered to the subject in need thereof as part of the methods described herein. In some embodiments, the method comprises administering to the subject a dose of about 5 mg to about 1000 mg of the compound of Formula (I) once or twice daily.

In some embodiments, the method comprises administering to the subject a dose of about 5 mg to about 500 mg, about 5 mg to about 490 mg, about 5 mg to about 480 mg, about 5 mg to about 470 mg, about 5 mg to about 460 mg, about 5 mg to about 450 mg, about 5 mg to about 440 mg, about 5 mg to about 430 mg, about 5 mg to about 420 mg, about 5 mg to about 410 mg, about 5 mg to about 400 mg, about 5 mg to about 390 mg, about 5 mg to about 380 mg, about 5 mg to about 370 mg, about 5 mg to about 360 mg, about 5 mg to about 350 mg, about 5 mg to about 340 mg, about 5 mg to about 330 mg, about 5 mg to about 320 mg, about 5 mg to about 310 mg, about 5 mg to about 300 mg, about 5 mg to about 290 mg, about 5 mg to about 280 mg, about 5 mg to about 270 mg, about 5 mg to about 260 mg, about 5 mg to about 250 mg, about 5 mg to about 240 mg, about 5 mg to about 230 mg, about 5 mg to about 220 mg, about 5 mg to about 210 mg, about 5 mg to about 200 mg, about 5 mg to about 190 mg, about 5 mg to about 180 mg, about 5 mg to about 170 mg, about 5 mg to about 160 mg, about 5 mg to about 150 mg, about 5 mg to about 140 mg, about 5 mg to about 130 mg, about 5 mg to about 120 mg, about 5 mg to about 110 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 10 mg, about 10 mg to about 500 mg, about 10 mg to about 490 mg, about 10 mg to about 480 mg, about 10 mg to about 470 mg, about 10 mg to about 460 mg, about 10 mg to about 450 mg, about 10 mg to about 440 mg, about 10 mg to about 430 mg, about 10 mg to about 420 mg, about 10 mg to about 410 mg, about 10 mg to about 400 mg, about 10 mg to about 390 mg, about 10 mg to about 380 mg, about 10 mg to about 370 mg, about 10 mg to about 360 mg, about 10 mg to about 350 mg, about 10 mg to about 340 mg, about 10 mg to about 330 mg, about 10 mg to about 320 mg, about 10 mg to about 310 mg, about 10 mg to about 300 mg, about 10 mg to about 290 mg, about 10 mg to about 280 mg, about 10 mg to about 270 mg, about 10 mg to about 260 mg, about 10 mg to about 250 mg, about 10 mg to about 240 mg, about 10 mg to about 230 mg, about 10 mg to about 220 mg, about 10 mg to about 210 mg, about 10 mg to about 200 mg, about 10 mg to about 190 mg, about 10 mg to about 180 mg, about 10 mg to about 170 mg, about 10 mg to about 160 mg, about 10 mg to about 150 mg, about 10 mg to about 140 mg, about 10 mg to about 130 mg, about 10 mg to about 120 mg, about 10 mg to about 110 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 500 mg, about 20 mg to about 490 mg, about 20 mg to about 480 mg, about 20 mg to about 470 mg, about 20 mg to about 460 mg, about 20 mg to about 450 mg, about 20 mg to about 440 mg, about 20 mg to about 430 mg, about 20 mg to about 420 mg, about 20 mg to about 410 mg, about 20 mg to about 400 mg, about 20 mg to about 390 mg, about 20 mg to about 380 mg, about 20 mg to about 370 mg, about 20 mg to about 360 mg, about 20 mg to about 350 mg, about 20 mg to about 340 mg, about 20 mg to about 330 mg, about 20 mg to about 320 mg, about 20 mg to about 310 mg, about 20 mg to about 300 mg, about 20 mg to about 290 mg, about 20 mg to about 280 mg, about 20 mg to about 270 mg, about 20 mg to about 260 mg, about 20 mg to about 250 mg, about 20 mg to about 240 mg, about 20 mg to about 230 mg, about 20 mg to about 220 mg, about 20 mg to about 210 mg, about 20 mg to about 200 mg, about 20 mg to about 190 mg, about 20 mg to about 180 mg, about 20 mg to about 170 mg, about 20 mg to about 160 mg, about 20 mg to about 150 mg, about 20 mg to about 140 mg, about 20 mg to about 130 mg, about 20 mg to about 120 mg, about 20 mg to about 110 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 500 mg, about 30 mg to about 490 mg, about 30 mg to about 480 mg, about 30 mg to about 470 mg, about 30 mg to about 460 mg, about 30 mg to about 450 mg, about 30 mg to about 440 mg, about 30 mg to about 430 mg, about 30 mg to about 420 mg, about 30 mg to about 410 mg, about 30 mg to about 400 mg, about 30 mg to about 390 mg, about 30 mg to about 380 mg, about 30 mg to about 370 mg, about 30 mg to about 360 mg, about 30 mg to about 350 mg, about 30 mg to about 340 mg, about 30 mg to about 330 mg, about 30 mg to about 320 mg, about 30 mg to about 310 mg, about 30 mg to about 300 mg, about 30 mg to about 290 mg, about 30 mg to about 280 mg, about 30 mg to about 270 mg, about 30 mg to about 260 mg, about 30 mg to about 250 mg, about 30 mg to about 240 mg, about 30 mg to about 230 mg, about 30 mg to about 220 mg, about 30 mg to about 210 mg, about 30 mg to about 200 mg, about 30 mg to about 190 mg, about 30 mg to about 180 mg, about 30 mg to about 170 mg, about 30 mg to about 160 mg, about 30 mg to about 150 mg, about 30 mg to about 140 mg, about 30 mg to about 130 mg, about 30 mg to about 120 mg, about 30 mg to about 110 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 500 mg, about 40 mg to about 490 mg, about 40 mg to about 480 mg, about 40 mg to about 470 mg, about 40 mg to about 460 mg, about 40 mg to about 450 mg, about 40 mg to about 440 mg, about 40 mg to about 430 mg, about 40 mg to about 420 mg, about 40 mg to about 410 mg, about 40 mg to about 400 mg, about 40 mg to about 390 mg, about 40 mg to about 380 mg, about 40 mg to about 370 mg, about 40 mg to about 360 mg, about 40 mg to about 350 mg, about 40 mg to about 340 mg, about 40 mg to about 330 mg, about 40 mg to about 320 mg, about 40 mg to about 310 mg, about 40 mg to about 300 mg, about 40 mg to about 290 mg, about 40 mg to about 280 mg, about 40 mg to about 270 mg, about 40 mg to about 260 mg, about 40 mg to about 250 mg, about 40 mg to about 240 mg, about 40 mg to about 230 mg, about 40 mg to about 220 mg, about 40 mg to about 210 mg, about 40 mg to about 200 mg, about 40 mg to about 190 mg, about 40 mg to about 180 mg, about 40 mg to about 170 mg, about 40 mg to about 160 mg, about 40 mg to about 150 mg, about 40 mg to about 140 mg, about 40 mg to about 130 mg, about 40 mg to about 120 mg, about 40 mg to about 110 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 500 mg, about 50 mg to about 490 mg, about 50 mg to about 480 mg, about 50 mg to about 470 mg, about 50 mg to about 460 mg, about 50 mg to about 450 mg, about 50 mg to about 440 mg, about 50 mg to about 430 mg, about 50 mg to about 420 mg, about 50 mg to about 410 mg, about 50 mg to about 400 mg, about 50 mg to about 390 mg, about 50 mg to about 380 mg, about 50 mg to about 370 mg, about 50 mg to about 360 mg, about 50 mg to about 350 mg, about 50 mg to about 340 mg, about 50 mg to about 330 mg, about 50 mg to about 320 mg, about 50 mg to about 310 mg, about 50 mg to about 300 mg, about 50 mg to about 290 mg, about 50 mg to about 280 mg, about 50 mg to about 270 mg, about 50 mg to about 260 mg, about 50 mg to about 250 mg, about 50 mg to about 240 mg, about 50 mg to about 230 mg, about 50 mg to about 220 mg, about 50 mg to about 210 mg, about 50 mg to about 200 mg, about 50 mg to about 190 mg, about 50 mg to about 180 mg, about 50 mg to about 170 mg, about 50 mg to about 160 mg, about 50 mg to about 150 mg, about 50 mg to about 140 mg, about 50 mg to about 130 mg, about 50 mg to about 120 mg, about 50 mg to about 110 mg, about 50 mg to about 100 mg, about 50 mg to about 90 mg, about 50 mg to about 80 mg, about 50 mg to about 70 mg, about 50 mg to about 60 mg, about 60 mg to about 500 mg, about 60 mg to about 490 mg, about 60 mg to about 480 mg, about 60 mg to about 470 mg, about 60 mg to about 460 mg, about 60 mg to about 450 mg, about 60 mg to about 440 mg, about 60 mg to about 430 mg, about 60 mg to about 420 mg, about 60 mg to about 410 mg, about 60 mg to about 400 mg, about 60 mg to about 390 mg, about 60 mg to about 380 mg, about 60 mg to about 370 mg, about 60 mg to about 360 mg, about 60 mg to about 350 mg, about 60 mg to about 340 mg, about 60 mg to about 330 mg, about 60 mg to about 320 mg, about 60 mg to about 310 mg, about 60 mg to about 300 mg, about 60 mg to about 290 mg, about 60 mg to about 280 mg, about 60 mg to about 270 mg, about 60 mg to about 260 mg, about 60 mg to about 250 mg, about 60 mg to about 240 mg, about 60 mg to about 230 mg, about 60 mg to about 220 mg, about 60 mg to about 210 mg, about 60 mg to about 200 mg, about 60 mg to about 190 mg, about 60 mg to about 180 mg, about 60 mg to about 170 mg, about 60 mg to about 160 mg, about 60 mg to about 150 mg, about 60 mg to about 140 mg, about 60 mg to about 130 mg, about 60 mg to about 120 mg, about 60 mg to about 110 mg, about 60 mg to about 100 mg, about 60 mg to about 90 mg, about 60 mg to about 80 mg, about 60 mg to about 70 mg, about 70 mg to about 500 mg, about 70 mg to about 490 mg, about 70 mg to about 480 mg, about 70 mg to about 470 mg, about 70 mg to about 460 mg, about 70 mg to about 450 mg, about 70 mg to about 440 mg, about 70 mg to about 430 mg, about 70 mg to about 420 mg, about 70 mg to about 410 mg, about 70 mg to about 400 mg, about 70 mg to about 390 mg, about 70 mg to about 380 mg, about 70 mg to about 370 mg, about 70 mg to about 360 mg, about 70 mg to about 350 mg, about 70 mg to about 340 mg, about 70 mg to about 330 mg, about 70 mg to about 320 mg, about 70 mg to about 310 mg, about 70 mg to about 300 mg, about 70 mg to about 290 mg, about 70 mg to about 280 mg, about 70 mg to about 270 mg, about 70 mg to about 260 mg, about 70 mg to about 250 mg, about 70 mg to about 240 mg, about 70 mg to about 230 mg, about 70 mg to about 220 mg, about 70 mg to about 210 mg, about 70 mg to about 200 mg, about 70 mg to about 190 mg, about 70 mg to about 180 mg, about 70 mg to about 170 mg, about 70 mg to about 160 mg, about 70 mg to about 150 mg, about 70 mg to about 140 mg, about 70 mg to about 130 mg, about 70 mg to about 120 mg, about 70 mg to about 110 mg, about 70 mg to about 100 mg, about 70 mg to about 90 mg, about 70 mg to about 80 mg, about 80 mg to about 500 mg, about 80 mg to about 490 mg, about 80 mg to about 480 mg, about 80 mg to about 470 mg, about 80 mg to about 460 mg, about 80 mg to about 450 mg, about 80 mg to about 440 mg, about 80 mg to about 430 mg, about 80 mg to about 420 mg, about 80 mg to about 410 mg, about 80 mg to about 400 mg, about 80 mg to about 390 mg, about 80 mg to about 380 mg, about 80 mg to about 370 mg, about 80 mg to about 360 mg, about 80 mg to about 350 mg, about 80 mg to about 340 mg, about 80 mg to about 330 mg, about 80 mg to about 320 mg, about 80 mg to about 310 mg, about 80 mg to about 300 mg, about 80 mg to about 290 mg, about 80 mg to about 280 mg, about 80 mg to about 270 mg, about 80 mg to about 260 mg, about 80 mg to about 250 mg, about 80 mg to about 240 mg, about 80 mg to about 230 mg, about 80 mg to about 220 mg, about 80 mg to about 210 mg, about 80 mg to about 200 mg, about 80 mg to about 190 mg, about 80 mg to about 180 mg, about 80 mg to about 170 mg, about 80 mg to about 160 mg, about 80 mg to about 150 mg, about 80 mg to about 140 mg, about 80 mg to about 130 mg, about 80 mg to about 120 mg, about 80 mg to about 110 mg, about 80 mg to about 100 mg, about 80 mg to about 90 mg, about 90 mg to about 500 mg, about 90 mg to about 490 mg, about 90 mg to about 480 mg, about 90 mg to about 470 mg, about 90 mg to about 460 mg, about 90 mg to about 450 mg, about 90 mg to about 440 mg, about 90 mg to about 430 mg, about 90 mg to about 420 mg, about 90 mg to about 410 mg, about 90 mg to about 400 mg, about 90 mg to about 390 mg, about 90 mg to about 380 mg, about 90 mg to about 370 mg, about 90 mg to about 360 mg, about 90 mg to about 350 mg, about 90 mg to about 340 mg, about 90 mg to about 330 mg, about 90 mg to about 320 mg, about 90 mg to about 310 mg, about 90 mg to about 300 mg, about 90 mg to about 290 mg, about 90 mg to about 280 mg, about 90 mg to about 270 mg, about 90 mg to about 260 mg, about 90 mg to about 250 mg, about 90 mg to about 240 mg, about 90 mg to about 230 mg, about 90 mg to about 220 mg, about 90 mg to about 210 mg, about 90 mg to about 200 mg, about 90 mg to about 190 mg, about 90 mg to about 180 mg, about 90 mg to about 170 mg, about 90 mg to about 160 mg, about 90 mg to about 150 mg, about 90 mg to about 140 mg, about 90 mg to about 130 mg, about 90 mg to about 120 mg, about 90 mg to about 110 mg, about 90 mg to about 100 mg, about 100 mg to about 500 mg, about 100 mg to about 490 mg, about 100 mg to about 480 mg, about 100 mg to about 470 mg, about 100 mg to about 460 mg, about 100 mg to about 450 mg, about 100 mg to about 440 mg, about 100 mg to about 430 mg, about 100 mg to about 420 mg, about 100 mg to about 410 mg, about 100 mg to about 400 mg, about 100 mg to about 390 mg, about 100 mg to about 380 mg, about 100 mg to about 370 mg, about 100 mg to about 360 mg, about 100 mg to about 350 mg, about 100 mg to about 340 mg, about 100 mg to about 330 mg, about 100 mg to about 320 mg, about 100 mg to about 310 mg, about 100 mg to about 300 mg, about 100 mg to about 290 mg, about 100 mg to about 280 mg, about 100 mg to about 270 mg, about 100 mg to about 260 mg, about 100 mg to about 250 mg, about 100 mg to about 240 mg, about 100 mg to about 230 mg, about 100 mg to about 220 mg, about 100 mg to about 210 mg, about 100 mg to about 200 mg, about 100 mg to about 190 mg, about 100 mg to about 180 mg, about 100 mg to about 170 mg, about 100 mg to about 160 mg, about 100 mg to about 150 mg, about 100 mg to about 140 mg, about 100 mg to about 130 mg, about 100 mg to about 120 mg, about 100 mg to about 110 mg, about 110 mg to about 500 mg, about 110 mg to about 490 mg, about 110 mg to about 480 mg, about 110 mg to about 470 mg, about 110 mg to about 460 mg, about 110 mg to about 450 mg, about 110 mg to about 440 mg, about 110 mg to about 430 mg, about 110 mg to about 420 mg, about 110 mg to about 410 mg, about 110 mg to about 400 mg, about 110 mg to about 390 mg, about 110 mg to about 380 mg, about 110 mg to about 370 mg, about 110 mg to about 360 mg, about 110 mg to about 350 mg, about 110 mg to about 340 mg, about 110 mg to about 330 mg, about 110 mg to about 320 mg, about 110 mg to about 310 mg, about 110 mg to about 300 mg, about 110 mg to about 290 mg, about 110 mg to about 280 mg, about 110 mg to about 270 mg, about 110 mg to about 260 mg, about 110 mg to about 250 mg, about 110 mg to about 240 mg, about 110 mg to about 230 mg, about 110 mg to about 220 mg, about 110 mg to about 210 mg, about 110 mg to about 200 mg, about 110 mg to about 190 mg, about 110 mg to about 180 mg, about 110 mg to about 170 mg, about 110 mg to about 160 mg, about 110 mg to about 150 mg, about 110 mg to about 140 mg, about 110 mg to about 130 mg, about 110 mg to about 120 mg, about 120 mg to about 500 mg, about 120 mg to about 490 mg, about 120 mg to about 480 mg, about 120 mg to about 470 mg, about 120 mg to about 460 mg, about 120 mg to about 450 mg, about 120 mg to about 440 mg, about 120 mg to about 430 mg, about 120 mg to about 420 mg, about 120 mg to about 410 mg, about 120 mg to about 400 mg, about 120 mg to about 390 mg, about 120 mg to about 380 mg, about 120 mg to about 370 mg, about 120 mg to about 360 mg, about 120 mg to about 350 mg, about 120 mg to about 340 mg, about 120 mg to about 330 mg, about 120 mg to about 320 mg, about 120 mg to about 310 mg, about 120 mg to about 300 mg, about 120 mg to about 290 mg, about 120 mg to about 280 mg, about 120 mg to about 270 mg, about 120 mg to about 260 mg, about 120 mg to about 250 mg, about 120 mg to about 240 mg, about 120 mg to about 230 mg, about 120 mg to about 220 mg, about 120 mg to about 210 mg, about 120 mg to about 200 mg, about 120 mg to about 190 mg, about 120 mg to about 180 mg, about 120 mg to about 170 mg, about 120 mg to about 160 mg, about 120 mg to about 150 mg, about 120 mg to about 140 mg, about 120 mg to about 130 mg, about 130 mg to about 500 mg, about 130 mg to about 490 mg, about 130 mg to about 480 mg, about 130 mg to about 470 mg, about 130 mg to about 460 mg, about 130 mg to about 450 mg, about 130 mg to about 440 mg, about 130 mg to about 430 mg, about 130 mg to about 420 mg, about 130 mg to about 410 mg, about 130 mg to about 400 mg, about 130 mg to about 390 mg, about 130 mg to about 380 mg, about 130 mg to about 370 mg, about 130 mg to about 360 mg, about 130 mg to about 350 mg, about 130 mg to about 340 mg, about 130 mg to about 330 mg, about 130 mg to about 320 mg, about 130 mg to about 310 mg, about 130 mg to about 300 mg, about 130 mg to about 290 mg, about 130 mg to about 280 mg, about 130 mg to about 270 mg, about 130 mg to about 260 mg, about 130 mg to about 250 mg, about 130 mg to about 240 mg, about 130 mg to about 230 mg, about 130 mg to about 220 mg, about 130 mg to about 210 mg, about 130 mg to about 200 mg, about 130 mg to about 190 mg, about 130 mg to about 180 mg, about 130 mg to about 170 mg, about 130 mg to about 160 mg, about 130 mg to about 150 mg, about 130 mg to about 140 mg, about 140 mg to about 500 mg, about 140 mg to about 490 mg, about 140 mg to about 480 mg, about 140 mg to about 470 mg, about 140 mg to about 460 mg, about 140 mg to about 450 mg, about 140 mg to about 440 mg, about 140 mg to about 430 mg, about 140 mg to about 420 mg, about 140 mg to about 410 mg, about 140 mg to about 400 mg, about 140 mg to about 390 mg, about 140 mg to about 380 mg, about 140 mg to about 370 mg, about 140 mg to about 360 mg, about 140 mg to about 350 mg, about 140 mg to about 340 mg, about 140 mg to about 330 mg, about 140 mg to about 320 mg, about 140 mg to about 310 mg, about 140 mg to about 300 mg, about 140 mg to about 290 mg, about 140 mg to about 280 mg, about 140 mg to about 270 mg, about 140 mg to about 260 mg, about 140 mg to about 250 mg, about 140 mg to about 240 mg, about 140 mg to about 230 mg, about 140 mg to about 220 mg, about 140 mg to about 210 mg, about 140 mg to about 200 mg, about 140 mg to about 190 mg, about 140 mg to about 180 mg, about 140 mg to about 170 mg, about 140 mg to about 160 mg, about 140 mg to about 150 mg, about 150 mg to about 500 mg, about 150 mg to about 490 mg, about 150 mg to about 480 mg, about 150 mg to about 470 mg, about 150 mg to about 460 mg, about 150 mg to about 450 mg, about 150 mg to about 440 mg, about 150 mg to about 430 mg, about 150 mg to about 420 mg, about 150 mg to about 410 mg, about 150 mg to about 400 mg, about 150 mg to about 390 mg, about 150 mg to about 380 mg, about 150 mg to about 370 mg, about 150 mg to about 360 mg, about 150 mg to about 350 mg, about 150 mg to about 340 mg, about 150 mg to about 330 mg, about 150 mg to about 320 mg, about 150 mg to about 310 mg, about 150 mg to about 300 mg, about 150 mg to about 290 mg, about 150 mg to about 280 mg, about 150 mg to about 270 mg, about 150 mg to about 260 mg, about 150 mg to about 250 mg, about 150 mg to about 240 mg, about 150 mg to about 230 mg, about 150 mg to about 220 mg, about 150 mg to about 210 mg, about 150 mg to about 200 mg, about 150 mg to about 190 mg, about 150 mg to about 180 mg, about 150 mg to about 170 mg, about 150 mg to about 160 mg, about 160 mg to about 500 mg, about 160 mg to about 490 mg, about 160 mg to about 480 mg, about 160 mg to about 470 mg, about 160 mg to about 460 mg, about 160 mg to about 450 mg, about 160 mg to about 440 mg, about 160 mg to about 430 mg, about 160 mg to about 420 mg, about 160 mg to about 410 mg, about 160 mg to about 400 mg, about 160 mg to about 390 mg, about 160 mg to about 380 mg, about 160 mg to about 370 mg, about 160 mg to about 360 mg, about 160 mg to about 350 mg, about 160 mg to about 340 mg, about 160 mg to about 330 mg, about 160 mg to about 320 mg, about 160 mg to about 310 mg, about 160 mg to about 300 mg, about 160 mg to about 290 mg, about 160 mg to about 280 mg, about 160 mg to about 270 mg, about 160 mg to about 260 mg, about 160 mg to about 250 mg, about 160 mg to about 240 mg, about 160 mg to about 230 mg, about 160 mg to about 220 mg, about 160 mg to about 210 mg, about 160 mg to about 200 mg, about 160 mg to about 190 mg, about 160 mg to about 180 mg, about 160 mg to about 170 mg, about 170 mg to about 500 mg, about 170 mg to about 490 mg, about 170 mg to about 480 mg, about 170 mg to about 470 mg, about 170 mg to about 460 mg, about 170 mg to about 450 mg, about 170 mg to about 440 mg, about 170 mg to about 430 mg, about 170 mg to about 420 mg, about 170 mg to about 410 mg, about 170 mg to about 400 mg, about 170 mg to about 390 mg, about 170 mg to about 380 mg, about 170 mg to about 370 mg, about 170 mg to about 360 mg, about 170 mg to about 350 mg, about 170 mg to about 340 mg, about 170 mg to about 330 mg, about 170 mg to about 320 mg, about 170 mg to about 310 mg, about 170 mg to about 300 mg, about 170 mg to about 290 mg, about 170 mg to about 280 mg, about 170 mg to about 270 mg, about 170 mg to about 260 mg, about 170 mg to about 250 mg, about 170 mg to about 240 mg, about 170 mg to about 230 mg, about 170 mg to about 220 mg, about 170 mg to about 210 mg, about 170 mg to about 200 mg, about 170 mg to about 190 mg, about 170 mg to about 180 mg, about 180 mg to about 500 mg, about 180 mg to about 490 mg, about 180 mg to about 480 mg, about 180 mg to about 470 mg, about 180 mg to about 460 mg, about 180 mg to about 450 mg, about 180 mg to about 440 mg, about 180 mg to about 430 mg, about 180 mg to about 420 mg, about 180 mg to about 410 mg, about 180 mg to about 400 mg, about 180 mg to about 390 mg, about 180 mg to about 380 mg, about 180 mg to about 370 mg, about 180 mg to about 360 mg, about 180 mg to about 350 mg, about 180 mg to about 340 mg, about 180 mg to about 330 mg, about 180 mg to about 320 mg, about 180 mg to about 310 mg, about 180 mg to about 300 mg, about 180 mg to about 290 mg, about 180 mg to about 280 mg, about 180 mg to about 270 mg, about 180 mg to about 260 mg, about 180 mg to about 250 mg, about 180 mg to about 240 mg, about 180 mg to about 230 mg, about 180 mg to about 220 mg, about 180 mg to about 210 mg, about 180 mg to about 200 mg, about 180 mg to about 190 mg, about 190 mg to about 500 mg, about 190 mg to about 490 mg, about 190 mg to about 480 mg, about 190 mg to about 470 mg, about 190 mg to about 460 mg, about 190 mg to about 450 mg, about 190 mg to about 440 mg, about 190 mg to about 430 mg, about 190 mg to about 420 mg, about 190 mg to about 410 mg, about 190 mg to about 400 mg, about 190 mg to about 390 mg, about 190 mg to about 380 mg, about 190 mg to about 370 mg, about 190 mg to about 360 mg, about 190 mg to about 350 mg, about 190 mg to about 340 mg, about 190 mg to about 330 mg, about 190 mg to about 320 mg, about 190 mg to about 310 mg, about 190 mg to about 300 mg, about 190 mg to about 290 mg, about 190 mg to about 280 mg, about 190 mg to about 270 mg, about 190 mg to about 260 mg, about 190 mg to about 250 mg, about 190 mg to about 240 mg, about 190 mg to about 230 mg, about 190 mg to about 220 mg, about 190 mg to about 210 mg, about 190 mg to about 200 mg, about 200 mg to about 500 mg, about 200 mg to about 490 mg, about 200 mg to about 480 mg, about 200 mg to about 470 mg, about 200 mg to about 460 mg, about 200 mg to about 450 mg, about 200 mg to about 440 mg, about 200 mg to about 430 mg, about 200 mg to about 420 mg, about 200 mg to about 410 mg, about 200 mg to about 400 mg, about 200 mg to about 390 mg, about 200 mg to about 380 mg, about 200 mg to about 370 mg, about 200 mg to about 360 mg, about 200 mg to about 350 mg, about 200 mg to about 340 mg, about 200 mg to about 330 mg, about 200 mg to about 320 mg, about 200 mg to about 310 mg, about 200 mg to about 300 mg, about 200 mg to about 290 mg, about 200 mg to about 280 mg, about 200 mg to about 270 mg, about 200 mg to about 260 mg, about 200 mg to about 250 mg, about 200 mg to about 240 mg, about 200 mg to about 230 mg, about 200 mg to about 220 mg, about 200 mg to about 210 mg, about 210 mg to about 500 mg, about 210 mg to about 490 mg, about 210 mg to about 480 mg, about 210 mg to about 470 mg, about 210 mg to about 460 mg, about 210 mg to about 450 mg, about 210 mg to about 440 mg, about 210 mg to about 430 mg, about 210 mg to about 420 mg, about 210 mg to about 410 mg, about 210 mg to about 400 mg, about 210 mg to about 390 mg, about 210 mg to about 380 mg, about 210 mg to about 370 mg, about 210 mg to about 360 mg, about 210 mg to about 350 mg, about 210 mg to about 340 mg, about 210 mg to about 330 mg, about 210 mg to about 320 mg, about 210 mg to about 310 mg, about 210 mg to about 300 mg, about 210 mg to about 290 mg, about 210 mg to about 280 mg, about 210 mg to about 270 mg, about 210 mg to about 260 mg, about 210 mg to about 250 mg, about 210 mg to about 240 mg, about 210 mg to about 230 mg, about 210 mg to about 220 mg, about 220 mg to about 500 mg, about 220 mg to about 490 mg, about 220 mg to about 480 mg, about 220 mg to about 470 mg, about 220 mg to about 460 mg, about 220 mg to about 450 mg, about 220 mg to about 440 mg, about 220 mg to about 430 mg, about 220 mg to about 420 mg, about 220 mg to about 410 mg, about 220 mg to about 400 mg, about 220 mg to about 390 mg, about 220 mg to about 380 mg, about 220 mg to about 370 mg, about 220 mg to about 360 mg, about 220 mg to about 350 mg, about 220 mg to about 340 mg, about 220 mg to about 330 mg, about 220 mg to about 320 mg, about 220 mg to about 310 mg, about 220 mg to about 300 mg, about 220 mg to about 290 mg, about 220 mg to about 280 mg, about 220 mg to about 270 mg, about 220 mg to about 260 mg, about 220 mg to about 250 mg, about 220 mg to about 240 mg, about 220 mg to about 230 mg, about 230 mg to about 500 mg, about 230 mg to about 490 mg, about 230 mg to about 480 mg, about 230 mg to about 470 mg, about 230 mg to about 460 mg, about 230 mg to about 450 mg, about 230 mg to about 440 mg, about 230 mg to about 430 mg, about 230 mg to about 420 mg, about 230 mg to about 410 mg, about 230 mg to about 400 mg, about 230 mg to about 390 mg, about 230 mg to about 380 mg, about 230 mg to about 370 mg, about 230 mg to about 360 mg, about 230 mg to about 350 mg, about 230 mg to about 340 mg, about 230 mg to about 330 mg, about 230 mg to about 320 mg, about 230 mg to about 310 mg, about 230 mg to about 300 mg, about 230 mg to about 290 mg, about 230 mg to about 280 mg, about 230 mg to about 270 mg, about 230 mg to about 260 mg, about 230 mg to about 250 mg, about 230 mg to about 240 mg, about 240 mg to about 500 mg, about 240 mg to about 490 mg, about 240 mg to about 480 mg, about 240 mg to about 470 mg, about 240 mg to about 460 mg, about 240 mg to about 450 mg, about 240 mg to about 440 mg, about 240 mg to about 430 mg, about 240 mg to about 420 mg, about 240 mg to about 410 mg, about 240 mg to about 400 mg, about 240 mg to about 390 mg, about 240 mg to about 380 mg, about 240 mg to about 370 mg, about 240 mg to about 360 mg, about 240 mg to about 350 mg, about 240 mg to about 340 mg, about 240 mg to about 330 mg, about 240 mg to about 320 mg, about 240 mg to about 310 mg, about 240 mg to about 300 mg, about 240 mg to about 290 mg, about 240 mg to about 280 mg, about 240 mg to about 270 mg, about 240 mg to about 260 mg, about 240 mg to about 250 mg, about 250 mg to about 500 mg, about 250 mg to about 490 mg, about 250 mg to about 480 mg, about 250 mg to about 470 mg, about 250 mg to about 460 mg, about 250 mg to about 450 mg, about 250 mg to about 440 mg, about 250 mg to about 430 mg, about 250 mg to about 420 mg, about 250 mg to about 410 mg, about 250 mg to about 400 mg, about 250 mg to about 390 mg, about 250 mg to about 380 mg, about 250 mg to about 370 mg, about 250 mg to about 360 mg, about 250 mg to about 350 mg, about 250 mg to about 340 mg, about 250 mg to about 330 mg, about 250 mg to about 320 mg, about 250 mg to about 310 mg, about 250 mg to about 300 mg, about 250 mg to about 290 mg, about 250 mg to about 280 mg, about 250 mg to about 270 mg, about 250 mg to about 260 mg, about 260 mg to about 500 mg, about 260 mg to about 490 mg, about 260 mg to about 480 mg, about 260 mg to about 470 mg, about 260 mg to about 460 mg, about 260 mg to about 450 mg, about 260 mg to about 440 mg, about 260 mg to about 430 mg, about 260 mg to about 420 mg, about 260 mg to about 410 mg, about 260 mg to about 400 mg, about 260 mg to about 390 mg, about 260 mg to about 380 mg, about 260 mg to about 370 mg, about 260 mg to about 360 mg, about 260 mg to about 350 mg, about 260 mg to about 340 mg, about 260 mg to about 330 mg, about 260 mg to about 320 mg, about 260 mg to about 310 mg, about 260 mg to about 300 mg, about 260 mg to about 290 mg, about 260 mg to about 280 mg, about 260 mg to about 270 mg, about 270 mg to about 500 mg, about 270 mg to about 490 mg, about 270 mg to about 480 mg, about 270 mg to about 470 mg, about 270 mg to about 460 mg, about 270 mg to about 450 mg, about 270 mg to about 440 mg, about 270 mg to about 430 mg, about 270 mg to about 420 mg, about 270 mg to about 410 mg, about 270 mg to about 400 mg, about 270 mg to about 390 mg, about 270 mg to about 380 mg, about 270 mg to about 370 mg, about 270 mg to about 360 mg, about 270 mg to about 350 mg, about 270 mg to about 340 mg, about 270 mg to about 330 mg, about 270 mg to about 320 mg, about 270 mg to about 310 mg, about 270 mg to about 300 mg, about 270 mg to about 290 mg, about 270 mg to about 280 mg, about 280 mg to about 500 mg, about 280 mg to about 490 mg, about 280 mg to about 480 mg, about 280 mg to about 470 mg, about 280 mg to about 460 mg, about 280 mg to about 450 mg, about 280 mg to about 440 mg, about 280 mg to about 430 mg, about 280 mg to about 420 mg, about 280 mg to about 410 mg, about 280 mg to about 400 mg, about 280 mg to about 390 mg, about 280 mg to about 380 mg, about 280 mg to about 370 mg, about 280 mg to about 360 mg, about 280 mg to about 350 mg, about 280 mg to about 340 mg, about 280 mg to about 330 mg, about 280 mg to about 320 mg, about 280 mg to about 310 mg, about 280 mg to about 300 mg, about 280 mg to about 290 mg, about 290 mg to about 500 mg, about 290 mg to about 490 mg, about 290 mg to about 480 mg, about 290 mg to about 470 mg, about 290 mg to about 460 mg, about 290 mg to about 450 mg, about 290 mg to about 440 mg, about 290 mg to about 430 mg, about 290 mg to about 420 mg, about 290 mg to about 410 mg, about 290 mg to about 400 mg, about 290 mg to about 390 mg, about 290 mg to about 380 mg, about 290 mg to about 370 mg, about 290 mg to about 360 mg, about 290 mg to about 350 mg, about 290 mg to about 340 mg, about 290 mg to about 330 mg, about 290 mg to about 320 mg, about 290 mg to about 310 mg, about 290 mg to about 300 mg, about 300 mg to about 500 mg, about 300 mg to about 490 mg, about 300 mg to about 480 mg, about 300 mg to about 470 mg, about 300 mg to about 460 mg, about 300 mg to about 450 mg, about 300 mg to about 440 mg, about 300 mg to about 430 mg, about 300 mg to about 420 mg, about 300 mg to about 410 mg, about 300 mg to about 400 mg, about 300 mg to about 390 mg, about 300 mg to about 380 mg, about 300 mg to about 370 mg, about 300 mg to about 360 mg, about 300 mg to about 350 mg, about 300 mg to about 340 mg, about 300 mg to about 330 mg, about 300 mg to about 320 mg, about 300 mg to about 310 mg, about 310 mg to about 500 mg, about 310 mg to about 490 mg, about 310 mg to about 480 mg, about 310 mg to about 470 mg, about 310 mg to about 460 mg, about 310 mg to about 450 mg, about 310 mg to about 440 mg, about 310 mg to about 430 mg, about 310 mg to about 420 mg, about 310 mg to about 410 mg, about 310 mg to about 400 mg, about 310 mg to about 390 mg, about 310 mg to about 380 mg, about 310 mg to about 370 mg, about 310 mg to about 360 mg, about 310 mg to about 350 mg, about 310 mg to about 340 mg, about 310 mg to about 330 mg, about 310 mg to about 320 mg, about 320 mg to about 500 mg, about 320 mg to about 490 mg, about 320 mg to about 480 mg, about 320 mg to about 470 mg, about 320 mg to about 460 mg, about 320 mg to about 450 mg, about 320 mg to about 440 mg, about 320 mg to about 430 mg, about 320 mg to about 420 mg, about 320 mg to about 410 mg, about 320 mg to about 400 mg, about 320 mg to about 390 mg, about 320 mg to about 380 mg, about 320 mg to about 370 mg, about 320 mg to about 360 mg, about 320 mg to about 350 mg, about 320 mg to about 340 mg, about 320 mg to about 330 mg, about 330 mg to about 500 mg, about 330 mg to about 490 mg, about 330 mg to about 480 mg, about 330 mg to about 470 mg, about 330 mg to about 460 mg, about 330 mg to about 450 mg, about 330 mg to about 440 mg, about 330 mg to about 430 mg, about 330 mg to about 420 mg, about 330 mg to about 410 mg, about 330 mg to about 400 mg, about 330 mg to about 390 mg, about 330 mg to about 380 mg, about 330 mg to about 370 mg, about 330 mg to about 360 mg, about 330 mg to about 350 mg, about 330 mg to about 340 mg, about 340 mg to about 500 mg, about 340 mg to about 490 mg, about 340 mg to about 480 mg, about 340 mg to about 470 mg, about 340 mg to about 460 mg, about 340 mg to about 450 mg, about 340 mg to about 440 mg, about 340 mg to about 430 mg, about 340 mg to about 420 mg, about 340 mg to about 410 mg, about 340 mg to about 400 mg, about 340 mg to about 390 mg, about 340 mg to about 380 mg, about 340 mg to about 370 mg, about 340 mg to about 360 mg, about 340 mg to about 350 mg, about 350 mg to about 500 mg, about 350 mg to about 490 mg, about 350 mg to about 480 mg, about 350 mg to about 470 mg, about 350 mg to about 460 mg, about 350 mg to about 450 mg, about 350 mg to about 440 mg, about 350 mg to about 430 mg, about 350 mg to about 420 mg, about 350 mg to about 410 mg, about 350 mg to about 400 mg, about 350 mg to about 390 mg, about 350 mg to about 380 mg, about 350 mg to about 370 mg, about 350 mg to about 360 mg, about 360 mg to about 500 mg, about 360 mg to about 490 mg, about 360 mg to about 480 mg, about 360 mg to about 470 mg, about 360 mg to about 460 mg, about 360 mg to about 450 mg, about 360 mg to about 440 mg, about 360 mg to about 430 mg, about 360 mg to about 420 mg, about 360 mg to about 410 mg, about 360 mg to about 400 mg, about 360 mg to about 390 mg, about 360 mg to about 380 mg, about 360 mg to about 370 mg, about 370 mg to about 500 mg, about 370 mg to about 490 mg, about 370 mg to about 480 mg, about 370 mg to about 470 mg, about 370 mg to about 460 mg, about 370 mg to about 450 mg, about 370 mg to about 440 mg, about 370 mg to about 430 mg, about 370 mg to about 420 mg, about 370 mg to about 410 mg, about 370 mg to about 400 mg, about 370 mg to about 390 mg, about 370 mg to about 380 mg, about 380 mg to about 500 mg, about 380 mg to about 490 mg, about 380 mg to about 480 mg, about 380 mg to about 470 mg, about 380 mg to about 460 mg, about 380 mg to about 450 mg, about 380 mg to about 440 mg, about 380 mg to about 430 mg, about 380 mg to about 420 mg, about 380 mg to about 410 mg, about 380 mg to about 400 mg, about 380 mg to about 390 mg, about 390 mg to about 500 mg, about 390 mg to about 490 mg, about 390 mg to about 480 mg, about 390 mg to about 470 mg, about 390 mg to about 460 mg, about 390 mg to about 450 mg, about 390 mg to about 440 mg, about 390 mg to about 430 mg, about 390 mg to about 420 mg, about 390 mg to about 410 mg, about 390 mg to about 400 mg, about 400 mg to about 500 mg, about 400 mg to about 490 mg, about 400 mg to about 480 mg, about 400 mg to about 470 mg, about 400 mg to about 460 mg, about 400 mg to about 450 mg, about 400 mg to about 440 mg, about 400 mg to about 430 mg, about 400 mg to about 420 mg, about 400 mg to about 410 mg, about 410 mg to about 500 mg, about 410 mg to about 490 mg, about 410 mg to about 480 mg, about 410 mg to about 470 mg, about 410 mg to about 460 mg, about 410 mg to about 450 mg, about 410 mg to about 440 mg, about 410 mg to about 430 mg, about 410 mg to about 420 mg, about 420 mg to about 500 mg, about 420 mg to about 490 mg, about 420 mg to about 480 mg, about 420 mg to about 470 mg, about 420 mg to about 460 mg, about 420 mg to about 450 mg, about 420 mg to about 440 mg, about 420 mg to about 430 mg, about 430 mg to about 500 mg, about 430 mg to about 490 mg, about 430 mg to about 480 mg, about 430 mg to about 470 mg, about 430 mg to about 460 mg, about 430 mg to about 450 mg, about 430 mg to about 440 mg, about 440 mg to about 500 mg, about 440 mg to about 490 mg, about 440 mg to about 480 mg, about 440 mg to about 470 mg, about 440 mg to about 460 mg, about 440 mg to about 450 mg, about 450 mg to about 500 mg, about 450 mg to about 490 mg, about 450 mg to about 480 mg, about 450 mg to about 470 mg, about 450 mg to about 460 mg, about 470 mg to about 500 mg, about 470 mg to about 490 mg, about 470 mg to about 480 mg, about 480 mg to about 500 mg, about 480 mg to about 490 mg or about 490 mg to about 500 mg of the compound of Formula (I) once or twice daily.

In some embodiments, the method comprises administering to the subject a dose of about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 200 mg, about 10 mg to about 150 mg, about 10 mg to about 120 mg, about 10 mg to about 100 mg, about 10 mg to about 50 mg, about 10 mg to about 20 mg, about 20 mg to about 500 mg, about 20 mg to about 450 mg, about 20 mg to about 400 mg, about 20 mg to about 350 mg, about 20 mg to about 320 mg, about 20 mg to about 300 mg, about 20 mg to about 250 mg, about 20 mg to about 200 mg, about 20 mg to about 150 mg, about 20 mg to about 120 mg, about 20 mg to about 100 mg, about 20 mg to about 50 mg, about 40 mg to about 500 mg, about 40 mg to about 450 mg, about 40 mg to about 400 mg, about 40 mg to about 350 mg, about 40 mg to about 320 mg, about 40 mg to about 300 mg, about 40 mg to about 250 mg, about 40 mg to about 200 mg, about 40 mg to about 150 mg, about 40 mg to about 120 mg, about 40 mg to about 100 mg, about 40 mg to about 50 mg, about 50 mg to about 500 mg, about 50 mg to about 450 mg, about 50 mg to about 400 mg, about 50 mg to about 350 mg, about 50 mg to about 320 mg, about 50 mg to about 300 mg, about 50 mg to about 250 mg, about 50 mg to about 200 mg, about 50 mg to about 150 mg, about 50 mg to about 120 mg, about 50 mg to about 100 mg, about 100 mg to about 500 mg, about 100 mg to about 450 mg, about 100 mg to about 400 mg, about 100 mg to about 350 mg, about 100 mg to about 320 mg, about 100 mg to about 300 mg, about 100 mg to about 250 mg, about 100 mg to about 200 mg, about 100 mg to about 150 mg, about 100 mg to about 120 mg, about 120 mg to about 500 mg, about 120 mg to about 450 mg, about 120 mg to about 400 mg, about 120 mg to about 350 mg, about 120 mg to about 320 mg, about 120 mg to about 300 mg, about 120 mg to about 250 mg, about 120 mg to about 200 mg, about 120 mg to about 150 mg, about 150 mg to about 500 mg, about 150 mg to about 450 mg, about 150 mg to about 400 mg, about 150 mg to about 350 mg, about 150 mg to about 320 mg, about 150 mg to about 300 mg, about 150 mg to about 250 mg, about 150 mg to about 200 mg, about 200 mg to about 500 mg, about 200 mg to about 450 mg, about 200 mg to about 400 mg, about 200 mg to about 350 mg, about 200 mg to about 320 mg, about 200 mg to about 300 mg, about 200 mg to about 250 mg, about 250 mg to about 500 mg, about 250 mg to about 450 mg, about 250 mg to about 400 mg, about 250 mg to about 350 mg, about 250 mg to about 320 mg, about 250 mg to about 300 mg, about 300 mg to about 500 mg, about 300 mg to about 450 mg, about 300 mg to about 400 mg, about 300 mg to about 350 mg, about 300 mg to about 320 mg, about 320 mg to about 500 mg, about 320 mg to about 450 mg, about 320 mg to about 400 mg, about 320 mg to about 350 mg, about 350 mg to about 500 mg, about 350 mg to about 450 mg, about 350 mg to about 400 mg, about 400 mg to about 500 mg, about 400 mg to about 450 mg or about 450 mg to about 500 mg of the compound of Formula (I) once or twice daily.

In some embodiments, the method comprises administering to the subject a dose of about 20 mg to about 400 mg, about 20 mg to about 350 mg, about 20 mg to about 320 mg, about 20 mg to about 300 mg, about 20 mg to about 250 mg, about 20 mg to about 200 mg, about 20 mg to about 150 mg, about 20 mg to about 120 mg, about 20 mg to about 100 mg, about 20 mg to about 50 mg, about 40 mg to about 400 mg, about 40 mg to about 350 mg, about 40 mg to about 320 mg, about 40 mg to about 300 mg, about 40 mg to about 250 mg, about 40 mg to about 200 mg, about 40 mg to about 150 mg, about 40 mg to about 120 mg, about 40 mg to about 100 mg, about 50 mg to about 400 mg, about 50 mg to about 350 mg, about 50 mg to about 320 mg, about 50 mg to about 300 mg, about 50 mg to about 250 mg, about 50 mg to about 200 mg, about 50 mg to about 150 mg, about 50 mg to about 120 mg, about 50 mg to about 100 mg, about 100 mg to about 400 mg, about 100 mg to about 350 mg, about 100 mg to about 320 mg, about 100 mg to about 300 mg, about 100 mg to about 250 mg, about 100 mg to about 200 mg, about 100 mg to about 150 mg, about 120 mg to about 400 mg, about 120 mg to about 350 mg, about 120 mg to about 320 mg, about 120 mg to about 300 mg, about 120 mg to about 250 mg, about 120 mg to about 200 mg, about 150 mg to about 400 mg, about 150 mg to about 350 mg, about 150 mg to about 320 mg, about 150 mg to about 300 mg, about 150 mg to about 250 mg, about 150 mg to about 200 mg, about 200 mg to about 400 mg, about 200 mg to about 350 mg, about 200 mg to about 320 mg, about 200 mg to about 300 mg, about 200 mg to about 250 mg, about 250 mg to about 400 mg, about 250 mg to about 350 mg, about 250 mg to about 320 mg, about 250 mg to about 300 mg, about 300 mg to about 400 mg, about 300 mg to about 350 mg, about 320 mg to about 400 mg or about 350 mg to about 400 mg of the compound of Formula (I) once or twice daily.

In some embodiments, the method comprises administering to the subject a dose of about 10 mg to about 500 mg of the compound of Formula (I) once or twice daily. In some embodiments, the method comprises administering to the subject a dose of about 10 mg to about 400 mg of the compound of Formula (I) once or twice daily. In some embodiments, the method comprises administering to the subject a dose of about 10 mg to about 300 mg of the compound of Formula (I) once or twice daily. In some embodiments, the method comprises administering to the subject a dose of about 10 mg to about 200 mg of the compound of Formula (I) once or twice daily. In some embodiments, the method comprises administering to the subject a dose of about 10 mg to about 100 mg of the compound of Formula (I) once or twice daily. In some embodiments, the method comprises administering to the subject a dose of about 20 mg to about 500 mg of the compound of Formula (I) once or twice daily. In some embodiments, the method comprises administering to the subject a dose of about 20 mg to about 400 mg of the compound of Formula (I) once or twice daily. In some embodiments, the method comprises administering to the subject a dose of about 20 mg to about 300 mg of the compound of Formula (I) once or twice daily. In some embodiments, the method comprises administering to the subject a dose of about 20 mg to about 200 mg of the compound of Formula (I) once or twice daily. In some embodiments, the method comprises administering to the subject a dose of about 20 mg to about 100 mg of the compound of Formula (I) once or twice daily. In some embodiments, the method comprises administering to the subject a dose of about 50 mg to about 500 mg of the compound of Formula (I) once or twice daily. In some embodiments, the method comprises administering to the subject a dose of about 50 mg to about 400 mg of the compound of Formula (I) once or twice daily. In some embodiments, the method comprises administering to the subject a dose of about 50 mg to about 300 mg of the compound of Formula (I) once or twice daily. In some embodiments, the method comprises administering to the subject a dose of about 50 mg to about 200 mg of the compound of Formula (I) once or twice daily. In some embodiments, the method comprises administering to the subject a dose of about 50 mg to about 100 mg of the compound of Formula (I) once or twice daily. In some embodiments, the method comprises administering to the subject a dose of about 100 mg to about 500 mg of the compound of Formula (I) once or twice daily. In some embodiments, the method comprises administering to the subject a dose of about 100 mg to about 400 mg of the compound of Formula (I) once or twice daily. In some embodiments, the method comprises administering to the subject a dose of about 100 mg to about 300 mg of the compound of Formula (I) once or twice daily. In some embodiments, the method comprises administering to the subject a dose of about 100 mg to about 200 mg of the compound of Formula (I) once or twice daily. In some embodiments, the method comprises administering to the subject a dose of about 200 mg to about 500 mg of the compound of Formula (I) once or twice daily. In some embodiments, the method comprises administering to the subject a dose of about 200 mg to about 400 mg of the compound of Formula (I) once or twice daily. In some embodiments, the method comprises administering to the subject a dose of about 200 mg to about 300 mg of the compound of Formula (I) once or twice daily. In some embodiments, the method comprises administering to the subject a dose of about 300 mg to about 500 mg of the compound of Formula (I) once or twice daily. In some embodiments, the method comprises administering to the subject a dose of about 300 mg to about 400 mg of the compound of Formula (I) once or twice daily.

In some embodiments, the method comprises administering to the subject a dose of about 15 mg to about 25 mg of the compound of Formula (I) once or twice daily. In some embodiments, the method comprises administering to the subject a dose of about 18 mg to about 22 mg of the compound of Formula (I) once or twice daily. In some embodiments, the method comprises administering to the subject a dose of about 19 mg to about 21 mg of the compound of Formula (I) once or twice daily.

In some embodiments, the method comprises administering to the subject a dose of about 15 mg, about 16 mg, about 17 mg, 18 mg, about 19 mg, about 20 mg, about 21 mg, about 22 mg, about 23 mg, about 24 mg, or about 25 mg of the compound of Formula (I) once or twice daily.

In some embodiments, the method comprises administering to the subject a dose of about 40 mg to about 60 mg of the compound of Formula (I) once or twice daily. In some embodiments, the method comprises administering to the subject a dose of about 45 mg to about 55 mg of the compound of Formula (I) once or twice daily. In some embodiments, the method comprises administering to the subject a dose of about 47 mg to about 53 mg of the compound of Formula (I) once or twice daily. In some embodiments, the method comprises administering to the subject a dose of about 49 mg to about 51 mg of the compound of Formula (I) once or twice daily.

In some embodiments, the method comprises administering to the subject a dose of about 45 mg, about 46 mg, about 47 mg, about 48 mg, about 49 mg, about 50 mg, about 51 mg, about 52 mg, about 53 mg, about 54 mg or about 55 mg of the compound of Formula (I) once or twice daily.

In some embodiments, the method comprises administering to the subject a dose of about 15 mg, about 16 mg, about 17 mg, 18 mg, about 19 mg, about 20 mg, about 21 mg, about 22 mg, about 23 mg, about 24 mg, about 25 mg, about 45 mg, about 46 mg, about 47 mg, about 48 mg, about 49 mg, about 50 mg, about 51 mg, about 52 mg, about 53 mg, about 54 mg or about 55 mg of the compound of Formula (I) once or twice daily.

In some embodiments, the method comprises administering to the subject a dose of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg or about 500 mg of the compound of Formula (I) once or twice daily. In some embodiments, the method comprises administering to the subject a dose of about 10 mg, about 20 mg, about 50 mg, about 60 mg, about 100 mg, about 120 mg, about 150 mg, about 200 mg, about 250 mg, about 300 mg, about 320 mg, about 350 mg, about 400 mg, about 450 mg or about 500 mg of the compound of Formula (I) once or twice daily. In some embodiments, the method comprises administering to the subject a dose of about 20 mg, about 40 mg, about 50 mg, about 60 mg, about 80 mg, about 100 mg, about 120 mg, about 150 mg, about 200 mg, about 250 mg, about 300 mg, about 320 mg, about 350 mg, about 400 mg, about 450 mg or about 500 mg of the compound of Formula (I) once or twice daily.

In some embodiments, the method comprises administering to the subject a dose of about 20 mg or about 50 mg of the compound of Formula (I) once or twice daily.

In an embodiment, the method comprises administering to the subject a dose of about 10 mg of the compound of Formula (I) once or twice daily. In an embodiment, the method comprises administering to the subject a dose of about 20 mg of the compound of Formula (I) once or twice daily. In an embodiment, the method comprises administering to the subject a dose of about 30 mg of the compound of Formula (I) once or twice daily. In an embodiment, the method comprises administering to the subject a dose of about 40 mg of the compound of Formula (I) once or twice daily. In an embodiment, the method comprises administering to the subject a dose of about 50 mg of the compound of Formula (I) once or twice daily. In an embodiment, the method comprises administering to the subject a dose of about 60 mg of the compound of Formula (I) once or twice daily. In an embodiment, the method comprises administering to the subject a dose of about 70 mg of the compound of Formula (I) once or twice daily. In an embodiment, the method comprises administering to the subject a dose of about 80 mg of the compound of Formula (I) once or twice daily. In an embodiment, the method comprises administering to the subject a dose of about 90 mg of the compound of Formula (I) once or twice daily. In an embodiment, the method comprises administering to the subject a dose of about 100 mg of the compound of Formula (I) once or twice daily. In an embodiment, the method comprises administering to the subject a dose of about 110 mg of the compound of Formula (I) once or twice daily. In an embodiment, the method comprises administering to the subject a dose of about 120 mg of the compound of Formula (I) once or twice daily. In an embodiment, the method comprises administering to the subject a dose of about 130 mg of the compound of Formula (I) once or twice daily. In an embodiment, the method comprises administering to the subject a dose of about 140 mg of the compound of Formula (I) once or twice daily. In an embodiment, the method comprises administering to the subject a dose of about 150 mg of the compound of Formula (I) once or twice daily. In an embodiment, the method comprises administering to the subject a dose of about 160 mg of the compound of Formula (I) once or twice daily. In an embodiment, the method comprises administering to the subject a dose of about 170 mg of the compound of Formula (I) once or twice daily. In an embodiment, the method comprises administering to the subject a dose of about 180 mg of the compound of Formula (I) once or twice daily. In an embodiment, the method comprises administering to the subject a dose of about 190 mg of the compound of Formula (I) once or twice daily. In an embodiment, the method comprises administering to the subject a dose of about 200 mg of the compound of Formula (I) once or twice daily. In an embodiment, the method comprises administering to the subject a dose of about 210 mg of the compound of Formula (I) once or twice daily. In an embodiment, the method comprises administering to the subject a dose of about 220 mg of the compound of Formula (I) once or twice daily. In an embodiment, the method comprises administering to the subject a dose of about 230 mg of the compound of Formula (I) once or twice daily. In an embodiment, the method comprises administering to the subject a dose of about 240 mg of the compound of Formula (I) once or twice daily. In an embodiment, the method comprises administering to the subject a dose of about 250 mg of the compound of Formula (I) once or twice daily. In an embodiment, the method comprises administering to the subject a dose of about 260 mg of the compound of Formula (I) once or twice daily. In an embodiment, the method comprises administering to the subject a dose of about 270 mg of the compound of Formula (I) once or twice daily. In an embodiment, the method comprises administering to the subject a dose of about 280 mg of the compound of Formula (I) once or twice daily. In an embodiment, the method comprises administering to the subject a dose of about 290 mg of the compound of Formula (I) once or twice daily. In an embodiment, the method comprises administering to the subject a dose of about 300 mg of the compound of Formula (I) once or twice daily. In an embodiment, the method comprises administering to the subject a dose of about 310 mg of the compound of Formula (I) once or twice daily. In an embodiment, the method comprises administering to the subject a dose of about 320 mg of the compound of Formula (I) once or twice daily. In an embodiment, the method comprises administering to the subject a dose of about 330 mg of the compound of Formula (I) once or twice daily. In an embodiment, the method comprises administering to the subject a dose of about 340 mg of the compound of Formula (I) once or twice daily. In an embodiment, the method comprises administering to the subject a dose of about 350 mg of the compound of Formula (I) once or twice daily. In an embodiment, the method comprises administering to the subject a dose of about 360 mg of the compound of Formula (I) once or twice daily. In an embodiment, the method comprises administering to the subject a dose of about 370 mg of the compound of Formula (I) once or twice daily. In an embodiment, the method comprises administering to the subject a dose of about 380 mg of the compound of Formula (I) once or twice daily. In an embodiment, the method comprises administering to the subject a dose of about 390 mg of the compound of Formula (I) once or twice daily. In an embodiment, the method comprises administering to the subject a dose of about 400 mg of the compound of Formula (I) once or twice daily. In an embodiment, the method comprises administering to the subject a dose of about 410 mg of the compound of Formula (I) once or twice daily. In an embodiment, the method comprises administering to the subject a dose of about 420 mg of the compound of Formula (I) once or twice daily. In an embodiment, the method comprises administering to the subject a dose of about 430 mg of the compound of Formula (I) once or twice daily. In an embodiment, the method comprises administering to the subject a dose of about 440 mg of the compound of Formula (I) once or twice daily. In an embodiment, the method comprises administering to the subject a dose of about 450 mg of the compound of Formula (I) once or twice daily. In an embodiment, the method comprises administering to the subject a dose of about 460 mg of the compound of Formula (I) once or twice daily. In an embodiment, the method comprises administering to the subject a dose of about 470 mg of the compound of Formula (I) once or twice daily. In an embodiment, the method comprises administering to the subject a dose of about 480 mg of the compound of Formula (I) once or twice daily. In an embodiment, the method comprises administering to the subject a dose of about 490 mg of the compound of Formula (I) once or twice daily. In an embodiment, the method comprises administering to the subject a dose of about 500 mg of the compound of Formula (I) once or twice daily.

In some embodiments, the method comprises administering the dose of the compound of Formula (I) once daily (e.g., every 24 hours).

In some embodiments, the method comprises administering to the subject a dose of about 15 mg to about 25 mg of the compound of Formula (I) once daily (e.g., every 24 hours). In some embodiments, the method comprises administering to the subject a dose of about 18 mg to about 22 mg of the compound of Formula (I) once daily (e.g., every 24 hours).

In some embodiments, the method comprises administering to the subject a dose of about 15 mg, about 16 mg, about 17 mg, about 18 mg, about 19 mg, about 20 mg, about 21 mg, about 22 mg, about 23 mg, about 24 mg or about 25 mg of the compound of Formula (I) once daily (e.g., every 24 hours).

In further embodiments, the method comprises administering to the subject a dose of about 20 mg of the compound of Formula (I) once daily (e.g., every 24 hours).

In some embodiments, the method comprises administering to the subject a dose of about 40 mg to about 60 mg of the compound of Formula (I) once daily (e.g., every 24 hours). In some embodiments, the method comprises administering to the subject a dose of about 45 mg to about 55 mg of the compound of Formula (I) once daily (e.g., every 24 hours). In some embodiments, the method comprises administering to the subject a dose of about 47 mg to about 53 mg of the compound of Formula (I) once daily (e.g., every 24 hours). In some embodiments, the method comprises administering to the subject a dose of about 49 mg to about 51 mg of the compound of Formula (I) once daily (e.g., every 24 hours).

In some embodiments, the method comprises administering to the subject a dose of about 45 mg, about 46 mg, about 47 mg, about 48 mg, about 49 mg, about 50 mg, about 51 mg, about 52 mg, about 53 mg, about 54 mg or about 55 mg of the compound of Formula (I) once daily (e.g., every 24 hours).

In some embodiments, the method comprises administering to the subject a dose of about 50 mg of the compound of Formula (I) once daily (e.g., every 24 hours).

In some embodiments, the method comprises administering to the subject a dose of about 15 mg, about 16 mg, about 17 mg, about 18 mg, about 19 mg, about 20 mg, about 21 mg, about 22 mg, about 23 mg, about 24 mg, about 25 mg, about 45 mg, about 46 mg, about 47 mg, about 48 mg, about 49 mg, about 50 mg, about 51 mg, about 52 mg, about 53 mg, about 54 mg, about 55 mg, of the compound of Formula (I) once daily (e.g., every 24 hours).

In some embodiments, the method comprises administering to the subject a dose of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg or about 500 mg of the compound of Formula (I) once daily (e.g., every 24 hours). In some embodiments, the method comprises administering to the subject a dose of about 10 mg, about 20 mg, about 50 mg, about 60 mg, about 100 mg, about 120 mg, about 150 mg, about 200 mg, about 250 mg, about 300 mg, about 320 mg, about 350 mg, about 400 mg, about 450 mg or about 500 mg of the compound of Formula (I) once daily (e.g., every 24 hours).

In some embodiments, the method comprises administering the dose of the compound of Formula (I) twice daily.

In some embodiments, the method comprises administering the dose of the compound of Formula (I) twice daily (e.g., every 12 hours).

In some embodiments, the method comprises administering to the subject a dose of about 15 mg to about 25 mg of the compound of Formula (I) twice daily (e.g., every 12 hours). In some embodiments, the method comprises administering to the subject a dose of about 18 mg to about 22 mg of the compound of Formula (I) twice daily (e.g., every 12 hours).

In some embodiments, the method comprises administering to the subject a dose of about 15 mg, about 16 mg, about 17 mg, about 18 mg, about 19 mg, about 20 mg, about 21 mg, about 22 mg, about 23 mg, about 24 mg or about 25 mg of the compound of Formula (I) twice daily (e.g., every 12 hours).

In further embodiments, the method comprises administering to the subject a dose of about 20 mg of the compound of Formula (I) twice daily (e.g., every 12 hours).

In some embodiments, the method comprises administering to the subject a dose of about 40 mg to about 60 mg of the compound of Formula (I) twice daily (e.g., every 12 hours). In some embodiments, the method comprises administering to the subject a dose of about 45 mg to about 55 mg of the compound of Formula (I) twice daily (e.g., every 12 hours). In some embodiments, the method comprises administering to the subject a dose of about 47 mg to about 53 mg of the compound of Formula (I) twice daily (e.g., every 12 hours). In some embodiments, the method comprises administering to the subject a dose of about 49 mg to about 51 mg of the compound of Formula (I) twice daily (e.g., every 12 hours).

In some embodiments, the method comprises administering to the subject a dose of about 45 mg, about 46 mg, about 47 mg, about 48 mg, about 49 mg, about 50 mg, about 51 mg, about 52 mg, about 53 mg, about 54 mg or about 55 mg of the compound of Formula (I) twice daily (e.g., every 12 hours).

In some embodiments, the method comprises administering to the subject a dose of about 50 mg of the compound of Formula (I) twice daily (e.g., every 12 hours).

In some embodiments, the method comprises administering to the subject a dose of about 15 mg, about 16 mg, about 17 mg, about 18 mg, about 19 mg, about 20 mg, about 21 mg, about 22 mg, about 23 mg, about 24 mg, about 25 mg, about 45 mg, about 46 mg, about 47 mg, about 48 mg, about 49 mg, about 50 mg, about 51 mg, about 52 mg, about 53 mg, about 54 mg, about 55 mg, of the compound of Formula (I) twice daily (e.g., every 12 hours).

In some embodiments, the method comprises administering to the subject a dose of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg or about 500 mg of the compound of Formula (I) twice daily (e.g., every 12 hours). In some embodiments, the method comprises administering to the subject a dose of about 10 mg, about 20 mg, about 50 mg, about 60 mg, about 100 mg, about 120 mg, about 150 mg, about 200 mg, about 250 mg, about 300 mg, about 320 mg, about 350 mg, about 400 mg, about 450 mg or about 500 mg of the compound of Formula (I) twice daily (e.g., every 12 hours).

### Kits

In some embodiments provided are kits related to methods of use described herein.

In one embodiment, provided is a kit for predicting the sensitivity of a subject having or having been diagnosed with an MTAP-deficiency-related cancer for treatment with a PRMT5 inhibitor is provided. The kit comprises: i) reagents capable of detecting human MTAP-deficient and/or MTA-accumulating cancer cells; and ii) instructions for how to use said kit.

### SELECTED EMBODIMENTS

### Embodiment 1

A crystalline form of N-(6-amino-5-ethylpyridin-3-yl)-2-((2R,5S)-5-methyl-2-(2-(1-methylpiperidin-4-yl)benzo[d]thiazol-5-yl)piperidin-1-yl)-2-oxoacetamide (a Compound of Formula (I))

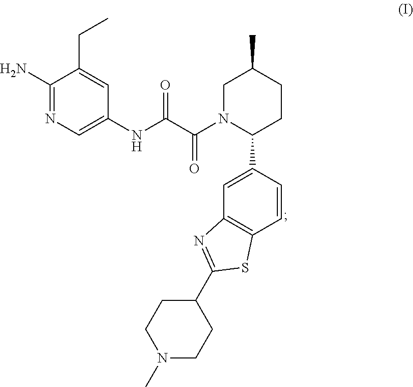 [SMILES: CCc1cc(NC(=O)C(=O)N2C[C@@H](C)CC[C@@H]2c2ccc3sc(C4CCN(C)CC4)nc3c2)cnc1N]

wherein the X-ray powder diffraction (XRPD) pattern of the crystalline form comprises one or more peaks at 2θ angles selected from 9.6±0.2, 16.8±0.2, 19.3±0.2, 19.4±0.2, 20.9±0.2, 23.7±0.2, 24.5±0.2 degrees.

### Embodiment 2

The crystalline form of embodiment 1, wherein the XRPD pattern of the crystalline form comprises one or more peaks at 2θ angles selected from 4.4±0.2, 9.6±0.2, 15.9±0.2, 16.8±0.2, 19.3±0.2, 19.3±0.2, 19.4±0.2, 20.9±0.2, 21.0±0.2, 23.7±0.2, 24.5±0.2 and 25.7±0.2, 34.0±0.2 degrees.

### Embodiment 3

The crystalline form of embodiment 1 wherein the crystalline form has an XRPD diffraction pattern substantially corresponding to the XRPD diffraction pattern shown in FIG. 1.

### Embodiment 4

The crystalline form of any one of embodiments 1 to 3 wherein crystalline form is substantially pure.

### Embodiment 5

A pharmaceutical composition comprising a compound of formula (I) and at least one pharmaceutically acceptable carrier, diluent, excipient or adjuvant.

### Embodiment 6

The pharmaceutical composition of embodiment 5 wherein the compound of formula (I) is a crystalline form of any one of embodiments 1 to 4.

### Embodiment 7

The pharmaceutical composition of embodiment 5 or 6, wherein the composition comprises about 10% (w/w) of the compound of formula (I).

### Embodiment 8

The pharmaceutical composition of embodiment 5 or 6, wherein the composition comprises about 2% (w/w) to about 20% (w/w) of a compound of formula (I).

### Embodiment 9

The pharmaceutical composition of embodiment 5 or 6, wherein the composition comprises about 5% (w/w) to about 15% (w/w) of a compound of formula (I).

### Embodiment 10

The pharmaceutical composition of embodiment 5 or 6, wherein the composition comprises about 8% (w/w) to about 12% (w/w) of a compound of formula (I).

### Embodiment 11

The pharmaceutical composition of any one of embodiments 5 to 10, wherein the pharmaceutical composition comprises a filler (e.g., microcrystalline cellulose).

### Embodiment 12

The pharmaceutical composition of any one of embodiments 5 to 11, wherein the pharmaceutical composition comprises a glidant (e.g., colloidal silicon dioxide).

### Embodiment 13

The pharmaceutical composition of any one of embodiments 5 to 125, wherein the pharmaceutical composition comprises a disintegrant (e.g., croscarmellose sodium).

### Embodiment 14

The pharmaceutical composition of any one of embodiments 5 to 13, wherein the pharmaceutical composition comprises a lubricant (e.g., magnesium stearate).

### Embodiment 15

A pharmaceutical composition comprising:
(a) a compound of formula (I)

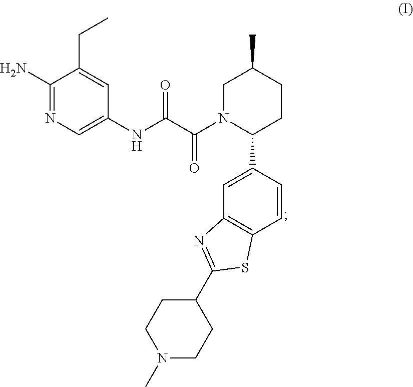 [SMILES: CCc1cc(NC(=O)C(=O)N2C[C@@H](C)CC[C@@H]2c2ccc3sc(C4CCN(C)CC4)nc3c2)cnc1N]

(b) a filler (e.g., microcrystalline cellulose);
(c) a glidant (e.g., colloidal silicon dioxide);
(d) a disintegrant (e.g., croscarmellose sodium); and
(e) a lubricant (e.g., magnesium stearate).

### Embodiment 16

The pharmaceutical composition of embodiment 15, wherein the composition comprises a crystalline form of the compound of formula (I) of any one of embodiments 1 to 4.

### Embodiment 17

The pharmaceutical composition of any one of embodiments 5 to 16, wherein the pharmaceutical composition comprises about 50% (w/w) to about 90% (w/w) filler.

### Embodiment 18

The pharmaceutical composition of any one of embodiments 5 to 16, wherein the pharmaceutical composition comprises about 75% (w/w) to about 86% (w/w) filler.

### Embodiment 19

The pharmaceutical composition of any one of embodiments 5 to 16, wherein the pharmaceutical composition comprises about 77% (w/w) to about 85% (w/w) filler.

### Embodiment 20

The pharmaceutical composition of any one of embodiments 5 to 16, wherein the pharmaceutical composition comprises about 80% (w/w) to about 85% (w/w) filler.

### Embodiment 21

The pharmaceutical composition of any one of embodiments 5 to 16, wherein the pharmaceutical composition comprises about 82% (w/w) to about 84% (w/w) filler.

### Embodiment 22

The pharmaceutical composition of any one of embodiments 5 to 16, wherein the pharmaceutical composition comprises about 75% (w/w), about 76% (w/w), about 77% (w/w), about 78% (w/w), about 79% (w/w), about 80% (w/w), about 81% (w/w), about 82% (w/w), about 83% (w/w), about 83.5% (w/w), about 84% (w/w), about 85% (w/w), about 86% (w/w) filler.

### Embodiment 23

The pharmaceutical composition of any one of embodiments 5 to 16, wherein the pharmaceutical composition comprises about 83.5% (w/w) filler.

### Embodiment 24

The pharmaceutical composition of any one of embodiments 5 to 23, wherein the pharmaceutical composition comprises about 0.5% (w/w) to about 2.5% (w/w) glidant.

### Embodiment 25

The pharmaceutical composition of any one of embodiments 5 to 23, wherein the pharmaceutical composition comprises about 0.75% (w/w) to about 2.25% (w/w) glidant.

### Embodiment 26

The pharmaceutical composition of any one of embodiments 5 to 23, wherein the pharmaceutical composition comprises about 1% (w/w) to about 2.0% (w/w) glidant.

### Embodiment 27

The pharmaceutical composition of any one of embodiments 5 to 23, wherein the pharmaceutical composition comprises about 1.2% (w/w) to about 1.8% (w/w) glidant.

### Embodiment 28

The pharmaceutical composition of any one of embodiments 5 to 23, wherein the pharmaceutical composition comprises about 1.3% (w/w) to about 1.7% (w/w) glidant.

### Embodiment 29

The pharmaceutical composition of any one of embodiments 5 to 23, wherein the pharmaceutical composition comprises about 1.45% (w/w) to about 1.55% (w/w) glidant.

### Embodiment 30

The pharmaceutical composition of any one of embodiments 5 to 23, wherein the pharmaceutical composition comprises about 1.4% (w/w), about 1.42% (w/w), about 1.44% (w/w), about 1.46% (w/w), about 1.48% (w/w), about 1.5% (w/w), about 1.52% (w/w), about 1.54% (w/w), about 1.56% (w/w), about 1.58% (w/w) or about 1.6% (w/w) glidant.

### Embodiment 31

The pharmaceutical composition of any one of embodiments 5 to 23, wherein the pharmaceutical composition comprises about 1.5% (w/w) glidant.

### Embodiment 32

The pharmaceutical composition of any one of embodiments 5 to 31, wherein the pharmaceutical composition comprises about 2% (w/w) to about 6% (w/w) disintegrant.

### Embodiment 33

The pharmaceutical composition of any one of embodiments 5 to 31, wherein the pharmaceutical composition comprises about 3% (w/w) to about 5% (w/w) disintegrant.

### Embodiment 34

The pharmaceutical composition of any one of embodiments 5 to 31, wherein the pharmaceutical composition comprises about 3.6% (w/w) to about 4.4% (w/w) disintegrant.

### Embodiment 35

The pharmaceutical composition of any one of embodiments 5 to 31, wherein the pharmaceutical composition comprises about 3.8% (w/w) to about 4.2% (w/w) disintegrant.

### Embodiment 36

The pharmaceutical composition of any one of embodiments 5 to 31, wherein the pharmaceutical composition comprises about 3.2% (w/w), about 3.28% (w/w), about 3.36% (w/w), about 3.44% (w/w), about 3.52% (w/w), about 3.6% (w/w), about 3.68% (w/w), about 3.76% (w/w), about 3.84% (w/w), about 3.92% (w/w), about 4% (w/w), about 4.08% (w/w), about 4.16% (w/w), about 4.24% (w/w), about 4.32% (w/w), about 4.4% (w/w), about 4.48% (w/w), about 4.56% (w/w), about 4.64% (w/w), about 4.72% (w/w), or about 4.8% (w/w) disintegrant.

### Embodiment 37

The pharmaceutical composition of any one of embodiments 5 to 31, wherein the pharmaceutical composition comprises about 3.84% (w/w), about 3.92% (w/w), about 4% (w/w), about 4.08% (w/w), about 4.16% (w/w), about 4.24% (w/w), about 4.32% (w/w), about 4.4% (w/w), about 4.48% (w/w), about 4.56% (w/w), about 4.64% (w/w), about 4.72% (w/w), or about 4.8% (w/w) disintegrant.

### Embodiment 38

The pharmaceutical composition of any one of embodiments 5 to 31, wherein the pharmaceutical composition comprises about 3.92% (w/w), about 4.00% (w/w) or about 4.08% (w/w) disintegrant.

### Embodiment 39

The pharmaceutical composition of any one of embodiments 5 to 31, wherein the pharmaceutical composition comprises about 4% (w/w) disintegrant.

### Embodiment 40

The pharmaceutical composition of any one of embodiments 5 to 39, wherein the pharmaceutical composition comprises about 0.5% (w/w) to about 1.5% (w/w) lubricant.

### Embodiment 41

The pharmaceutical composition of any one of embodiments 5 to 39, wherein the pharmaceutical composition comprises about 0.75% (w/w) to about 1.25% (w/w) lubricant.

### Embodiment 42

The pharmaceutical composition of any one of embodiments 5 to 39, wherein the pharmaceutical composition comprises about 0.9% (w/w) to about 1.1% (w/w) lubricant.

### Embodiment 43

The pharmaceutical composition of any one of embodiments 5 to 39, wherein the pharmaceutical composition comprises about 0.95% (w/w) to about 1.05% (w/w) lubricant.

### Embodiment 44

The pharmaceutical composition of any one of embodiments 5 to 39, wherein the pharmaceutical composition comprises about 0.9% (w/w), about 0.92% (w/w), about 0.94% (w/w), about 0.96% (w/w), about 0.98% (w/w), about 1.0% (w/w), about 1.02% (w/w), about 1.04% (w/w), about 1.06% (w/w), about 1.08% (w/w) or about 1.1% (w/w) lubricant.

### Embodiment 45

The pharmaceutical composition of any one of embodiments 5 to 39, wherein the pharmaceutical composition comprises about 0.98% (w/w), about 1.00% (w/w) or about 1.02% (w/w) lubricant.

### Embodiment 46

The pharmaceutical composition of any one of embodiments 5 to 39, wherein the pharmaceutical composition comprises about 1% (w/w) lubricant.

### Embodiment 47

A pharmaceutical composition of embodiment 15 or 16, wherein the composition comprises:
(a) about 2% (w/w) to about 20% (w/w) of the compound of formula (I);
(b) about 50% (w/w) to about 90% (w/w) of a filler (e.g., microcrystalline cellulose);
(c) about 0.5% (w/w) to about 2.5% (w/w) of a glidant (e.g., colloidal silicon dioxide);
(d) about 2% (w/w) to about 6% (w/w) of a disintegrant (e.g., croscarmellose sodium);
(e) about 0.5% (w/w) to about 1.5% (w/w) of a lubricant (e.g., magnesium stearate); thereby totaling no more than 100% (w/w) of the composition.

### Embodiment 48

A pharmaceutical composition of embodiment 15 or 16, wherein the composition comprises:
(a) about 5% (w/w) to about 15% (w/w) of the compound of formula (I);
(b) about 80% (w/w) to about 90% (w/w) of a filler (e.g., microcrystalline cellulose);
(c) about 1.4% (w/w) to about 1.6% (w/w) of a glidant (e.g., colloidal silicon dioxide);
(d) about 3.6% (w/w) to about 4.4% (w/w) of a disintegrant (e.g., croscarmellose sodium);
(e) about 0.9% (w/w) to about 1.1% (w/w) of a lubricant (e.g., magnesium stearate); thereby totaling no more than 100% (w/w) of the composition.

### Embodiment 49

A pharmaceutical composition of embodiment 15 or 16, wherein the composition comprises:
(a) about 8% (w/w) to about 12% (w/w) of the compound of formula (I);
(b) about 80% (w/w) to about 86% (w/w) of a filler (e.g., microcrystalline cellulose);
(c) about 1.45% (w/w) to about 1.55% (w/w) of a glidant (e.g., colloidal silicon dioxide);
(d) about 3.8% (w/w) to about 4.2% (w/w) of a disintegrant (e.g., croscarmellose sodium);
(e) about 0.95% (w/w) to about 1.05% (w/w) of a lubricant (e.g., magnesium stearate); thereby totaling no more than 100% (w/w) of the composition.

### Embodiment 50

A pharmaceutical composition of embodiment 15 or 16, wherein the composition comprises:
(a) about 10% (w/w) of the compound of formula (I);
(b) about 83.5% (w/w) of a filler (e.g., microcrystalline cellulose);
(c) about 1.5% (w/w) of a glidant (e.g., colloidal silicon dioxide);
(d) about 4% (w/w) of a disintegrant (e.g., croscarmellose sodium); and
(e) about 1% (w/w) of a lubricant (e.g., magnesium stearate);
thereby totaling no more than 100% (w/w) of the composition.

### Embodiment 51

The pharmaceutical composition of any one of embodiments 5 to 50 wherein the filler is microcrystalline cellulose.

### Embodiment 52

The pharmaceutical composition of any one of embodiments 5 to 50 wherein the filler is microcrystalline cellulose PH 102, PH 200 or a mixture thereof.

### Embodiment 53

The pharmaceutical composition of any one of embodiments 5 to 52 wherein the glidant is colloidal silicon dioxide.

### Embodiment 54

The pharmaceutical composition of any one of embodiments 5 to 53 wherein the disintegrant is croscarmellose sodium.

### Embodiment 55

The pharmaceutical composition of any one of embodiments 5 to 54 wherein the lubricant is magnesium stearate.

### Embodiment 56

The pharmaceutical composition of any one of embodiments 5 to 55, wherein the pharmaceutical composition comprises about 50% (w/w) to about 90% (w/w) intragranular filler.

### Embodiment 57

The pharmaceutical composition of any one of embodiments 5 to 55, wherein the pharmaceutical composition comprises about 75% (w/w) to about 86% (w/w) intragranular filler.

### Embodiment 58

The pharmaceutical composition of any one of embodiments 5 to 55, wherein the pharmaceutical composition comprises about 77% (w/w) to about 85% (w/w) intragranular filler.

### Embodiment 59

The pharmaceutical composition of any one of embodiments 5 to 55, wherein the pharmaceutical composition comprises about 80% (w/w) to about 85% (w/w) intragranular filler.

### Embodiment 60

The pharmaceutical composition of any one of embodiments 5 to 55, wherein the pharmaceutical composition comprises about 82% (w/w) to about 84% (w/w) intragranular filler.

### Embodiment 61

The pharmaceutical composition of any one of embodiments 5 to 55, wherein the pharmaceutical composition comprises about 75% (w/w), about 76% (w/w), about 77% (w/w), about 78% (w/w), about 79% (w/w), about 80% (w/w), about 81% (w/w), about 82% (w/w), about 83% (w/w), about 83.5% (w/w), about 84% (w/w), about 85% (w/w), about 86% (w/w) intragranular filler.

### Embodiment 62

The pharmaceutical composition of any one of embodiments 5 to 55, wherein the pharmaceutical composition comprises about 80% (w/w), about 81% (w/w), about 82% (w/w), about 83% (w/w), about 83.5% (w/w), about 84% (w/w), about 85% (w/w), about 86% (w/w) intragranular filler.

### Embodiment 63

The pharmaceutical composition of any one of embodiments 5 to 55, wherein the pharmaceutical composition comprises about 83.5% (w/w) intragranular filler.

### Embodiment 64

The pharmaceutical composition of any one of embodiments 5 to 63, wherein the pharmaceutical composition comprises about 0.75% (w/w) to about 1.25% (w/w) intragranular glidant.

### Embodiment 65

The pharmaceutical composition of any one of embodiments 5 to 63, wherein the pharmaceutical composition comprises about 0.8% (w/w) to about 1.2% (w/w) intragranular glidant.

### Embodiment 66

The pharmaceutical composition of any one of embodiments 5 to 63, wherein the pharmaceutical composition comprises about 0.85% (w/w) to about 1.15% (w/w) intragranular glidant.

### Embodiment 67

The pharmaceutical composition of any one of embodiments 5 to 63, wherein the pharmaceutical composition comprises about 0.9% (w/w) to about 1.1% (w/w) intragranular glidant.

### Embodiment 68

The pharmaceutical composition of any one of embodiments 5 to 63, wherein the pharmaceutical composition comprises about 0.95% (w/w) to about 1.05% (w/w) intragranular glidant.

### Embodiment 69

The pharmaceutical composition of any one of embodiments 5 to 63, wherein the pharmaceutical composition comprises about 0.97% (w/w) to about 1.3% (w/w) intragranular glidant.

### Embodiment 70

The pharmaceutical composition of any one of embodiments 5 to 63, wherein the pharmaceutical composition comprises about 0.9% (w/w), about 0.91% (w/w), about 0.92% (w/w), about 0.93% (w/w), about 0.94% (w/w), about 0.95% (w/w), about 0.96% (w/w), about 0.97% (w/w), about 0.98% (w/w), about 0.99% (w/w), about 1% (w/w), about 1.01% (w/w), about 1.02% (w/w), about 1.03% (w/w), about 1.04% (w/w), about 1.05% (w/w), about 1.06% (w/w), about 1.07% (w/w), about 1.08% (w/w), about 1.09% (w/w) or about 1.1% (w/w) intragranular glidant.

### Embodiment 71

The pharmaceutical composition of any one of embodiments 5 to 63, wherein the pharmaceutical composition comprises about 0.95% (w/w), about 0.96% (w/w), about 0.97% (w/w), about 0.98% (w/w), about 0.99% (w/w), about 1% (w/w), about 1.01% (w/w), about 1.02% (w/w), about 1.03% (w/w), about 1.04% (w/w) or about 1.05% (w/w) intragranular glidant.

### Embodiment 72

The pharmaceutical composition of any one of embodiments 5 to 63, wherein the pharmaceutical composition comprises about 1.0% (w/w) intragranular glidant.

### Embodiment 73

The pharmaceutical composition of any one of embodiments 5 to 72, wherein the pharmaceutical composition comprises about 0.25% (w/w) to about 0.75% (w/w) extragranular glidant.

### Embodiment 74

The pharmaceutical composition of any one of embodiments 5 to 72, wherein the pharmaceutical composition comprises about 0.3% (w/w) to about 0.7% (w/w) extragranular glidant.

### Embodiment 75

The pharmaceutical composition of any one of embodiments 5 to 72, wherein the pharmaceutical composition comprises about 0.4% (w/w) to about 0.6% (w/w) extragranular glidant.

### Embodiment 76

The pharmaceutical composition of any one of embodiments 5 to 72, wherein the pharmaceutical composition comprises about 0.45% (w/w) to about 0.55% (w/w) extragranular glidant.

### Embodiment 77

The pharmaceutical composition of any one of embodiments 5 to 72, wherein the pharmaceutical composition comprises about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) extragranular glidant.

### Embodiment 78

The pharmaceutical composition of any one of embodiments 5 to 72, wherein the pharmaceutical composition comprises about 0.48% (w/w), about 0.49% (w/w), about 0.5% (w/w), about 0.51% (w/w) or about 0.52% (w/w) extragranular glidant.

### Embodiment 79

The pharmaceutical composition of any one of embodiments 5 to 72, wherein the pharmaceutical composition comprises about 0.5% (w/w) extragranular glidant.

### Embodiment 80

The pharmaceutical composition of any one of embodiments 5 to 79, wherein the pharmaceutical composition comprises about 1% (w/w) to about 3% (w/w) intragranular disintegrant.

### Embodiment 81

The pharmaceutical composition of any one of embodiments 5 to 79, wherein the pharmaceutical composition comprises about 1.5% (w/w) to about 2.5% (w/w) intragranular disintegrant.

### Embodiment 82

The pharmaceutical composition of any one of embodiments 5 to 79, wherein the pharmaceutical composition comprises about 1.8% (w/w) to about 2.2% (w/w) intragranular disintegrant.

### Embodiment 83

The pharmaceutical composition of any one of embodiments 5 to 79, wherein the pharmaceutical composition comprises about 1.9% (w/w) to about 2.1% (w/w) intragranular disintegrant.

### Embodiment 84

The pharmaceutical composition of any one of embodiments 5 to 79, wherein the pharmaceutical composition comprises about 1.92% (w/w) to about 2.08% (w/w) intragranular disintegrant.

### Embodiment 85

The pharmaceutical composition of any one of embodiments 5 to 79, wherein the pharmaceutical composition comprises about 1.95% (w/w) to about 2.05% (w/w) intragranular disintegrant.

### Embodiment 86

The pharmaceutical composition of any one of embodiments 5 to 79, wherein the pharmaceutical composition comprises about 1.88% (w/w), about 1.92% (w/w), about 1.96% (w/w), about 2% (w/w), about 2.04% (w/w), about 2.08% (w/w), about 2.12% (w/w), about 2.16% (w/w) or about 2.2% (w/w) intragranular disintegrant.

### Embodiment 87

The pharmaceutical composition of any one of embodiments 5 to 79, wherein the pharmaceutical composition comprises about 1.92% (w/w), about 1.93% (w/w), about 1.94% (w/w), about 1.95% (w/w), about 1.96% (w/w), about 1.97% (w/w), about 1.98% (w/w), about 1.99% (w/w), about 2% (w/w), about 2.01% (w/w), about 2.02% (w/w), about 2.03% (w/w), about 2.04% (w/w), about 2.05% (w/w), about 2.06% (w/w), about 2.07% (w/w), about 2.08% (w/w), about 2.09% (w/w), about 2.1% (w/w), about 2.11% (w/w) or about 2.12% (w/w) intragranular disintegrant.

### Embodiment 88

The pharmaceutical composition of any one of embodiments 5 to 79, wherein the pharmaceutical composition comprises about 2% (w/w) intragranular disintegrant.

### Embodiment 89

The pharmaceutical composition of any one of embodiments 5 to 88, wherein the pharmaceutical composition comprises about 1% (w/w) to about 3% (w/w) extragranular disintegrant.

### Embodiment 90

The pharmaceutical composition of any one of embodiments 5 to 88, wherein the pharmaceutical composition comprises about 1.5% (w/w) to about 2.5% (w/w) extragranular disintegrant.

### Embodiment 91

The pharmaceutical composition of any one of embodiments 5 to 88, wherein the pharmaceutical composition comprises about 1.8% (w/w) to about 2.2% (w/w) extragranular disintegrant.

### Embodiment 92

The pharmaceutical composition of any one of embodiments 5 to 88, wherein the pharmaceutical composition comprises about 1.9% (w/w) to about 2.1% (w/w) extragranular disintegrant.

### Embodiment 93

The pharmaceutical composition of any one of embodiments 5 to 88, wherein the pharmaceutical composition comprises about 1.95% (w/w) to about 2.05% (w/w) extragranular disintegrant.

### Embodiment 94

The pharmaceutical composition of any one of embodiments 5 to 88, wherein the pharmaceutical composition comprises about 1.88% (w/w), about 1.92% (w/w), about 1.96% (w/w), about 2% (w/w), about 2.04% (w/w), about 2.08% (w/w), about 2.12% (w/w), about 2.16% (w/w) or about 2.2% (w/w) extragranular disintegrant.

### Embodiment 95

The pharmaceutical composition of any one of embodiments 5 to 88, wherein the pharmaceutical composition comprises about 1.92% (w/w), about 1.93% (w/w), about 1.94% (w/w), about 1.95% (w/w), about 1.96% (w/w), about 1.97% (w/w), about 1.98% (w/w), about 1.99% (w/w), about 2% (w/w), about 2.01% (w/w), about 2.02% (w/w), about 2.03% (w/w), about 2.04% (w/w), about 2.05% (w/w), about 2.06% (w/w), about 2.07% (w/w), about 2.08% (w/w), about 2.09% (w/w), about 2.1% (w/w), about 2.11% (w/w) or about 2.12% (w/w) extragranular disintegrant.

### Embodiment 96

The pharmaceutical composition of any one of embodiments 5 to 88, wherein the pharmaceutical composition comprises about 1.92% (w/w), about 1.96% (w/w), about 2% (w/w), about 2.04% (w/w) or about 2.08% (w/w) extragranular disintegrant.

### Embodiment 97

The pharmaceutical composition of any one of embodiments 5 to 88, wherein the pharmaceutical composition comprises about 2% (w/w) extragranular disintegrant.

### Embodiment 98

The pharmaceutical composition of any one of embodiments 5 to 97, wherein the pharmaceutical composition comprises about 0.25% (w/w) to about 0.75% (w/w) intragranular lubricant.

### Embodiment 99

The pharmaceutical composition of any one of embodiments 5 to 97, wherein the pharmaceutical composition comprises about 0.3% (w/w) to about 0.7% (w/w) intragranular lubricant.

### Embodiment 100

The pharmaceutical composition of any one of embodiments 5 to 97, wherein the pharmaceutical composition comprises about 0.35% (w/w) to about 0.65% (w/w) intragranular lubricant.

### Embodiment 101

The pharmaceutical composition of any one of embodiments 5 to 97, wherein the pharmaceutical composition comprises about 0.4% (w/w) to about 0.6% (w/w) intragranular lubricant.

### Embodiment 102

The pharmaceutical composition of any one of embodiments 5 to 97, wherein the pharmaceutical composition comprises about 0.45% (w/w) to about 0.55% (w/w) intragranular lubricant.

### Embodiment 103

The pharmaceutical composition of any one of embodiments 5 to 97, wherein the pharmaceutical composition comprises about 0.48% (w/w) to about 0.52% (w/w) intragranular lubricant.

### Embodiment 104

The pharmaceutical composition of any one of embodiments 5 to 97, wherein the pharmaceutical composition comprises about comprises about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) intragranular lubricant.

### Embodiment 105

The pharmaceutical composition of any one of embodiments 5 to 97, wherein the pharmaceutical composition comprises about 0.48% (w/w), about 0.49% (w/w), about 0.5% (w/w), about 0.51% (w/w) or about 0.52% (w/w) intragranular lubricant.

### Embodiment 106

The pharmaceutical composition of any one of embodiments 5 to 97, wherein the pharmaceutical composition comprises about 0.5% (w/w) intragranular lubricant.

### Embodiment 107

The pharmaceutical composition of any one of embodiments 5 to 106, wherein the pharmaceutical composition comprises about 0.25% (w/w) to about 0.75% (w/w) extragranular lubricant.

### Embodiment 108

The pharmaceutical composition of any one of embodiments 5 to 106, wherein the pharmaceutical composition comprises about 0.3% (w/w) to about 0.7% (w/w) extragranular lubricant.

### Embodiment 109

The pharmaceutical composition of any one of embodiments 5 to 106, wherein the pharmaceutical composition comprises about 0.4% (w/w) to about 0.6% (w/w) extragranular lubricant.

### Embodiment 110

The pharmaceutical composition of any one of embodiments 5 to 106, wherein the pharmaceutical composition comprises about 0.45% (w/w) to about 0.55% (w/w) extragranular lubricant.

### Embodiment 111

The pharmaceutical composition of any one of embodiments 5 to 106, wherein the pharmaceutical composition comprises about 0.45% (w/w), about 0.46% (w/w), about 0.47% (w/w), about 0.48% (w/w), about 0.49% (w/w), about 0.5% (w/w), about 0.51% (w/w), about 0.52% (w/w), about 0.53% (w/w), about 0.54% (w/w) or about 0.55% (w/w) extragranular lubricant.

### Embodiment 112

The pharmaceutical composition of any one of embodiments 5 to 106, wherein the pharmaceutical composition comprises about 0.48% (w/w), about 0.49% (w/w), about 0.5% (w/w), about 0.51% (w/w) or about 0.52% (w/w) extragranular lubricant.

### Embodiment 113

The pharmaceutical composition of any one of embodiments 5 to 106, wherein the pharmaceutical composition comprises about 0.5% (w/w) extragranular lubricant.

### Embodiment 114

A pharmaceutical composition comprising:
(a) a compound of formula (I)

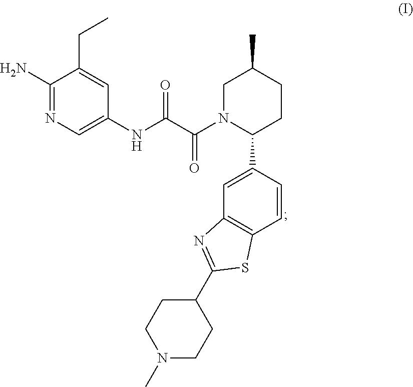 [SMILES: CCc1cc(NC(=O)C(=O)N2C[C@@H](C)CC[C@@H]2c2ccc3sc(C4CCN(C)CC4)nc3c2)cnc1N]

(b) an intragranular filler (e.g., microcrystalline cellulose);
(c) an intragranular glidant (e.g., colloidal silicon dioxide);
(d) an intragranular disintegrant (e.g., croscarmellose sodium);
(e) an intragranular lubricant (e.g., magnesium stearate);
(f) an extragranular glidant (e.g., colloidal silicon dioxide);
(g) an extragranular disintegrant (e.g., croscarmellose sodium); and
(h) an extragranular lubricant (e.g., magnesium stearate).

### Embodiment 115

The pharmaceutical composition of embodiment 114, wherein the composition comprises a crystalline form of the compound of formula (I) of any one of embodiments 1 to 4.

### Embodiment 116

A pharmaceutical composition of embodiment 114 or 115, wherein the composition comprises:
(a) about 2% (w/w) to about 20% (w/w) of the compound of formula (I) (e.g., crystalline form A);
(b) about 50% (w/w) to about 90% (w/w) of an intragranular filler (e.g., microcrystalline cellulose);
(c) about 0.75% (w/w) to about 1.25% (w/w) of an intragranular glidant (e.g., colloidal silicon dioxide);
(d) about 1% (w/w) to about 3% (w/w) of an intragranular disintegrant (e.g., croscarmellose sodium);
(e) about 0.25% (w/w) to about 0.75% (w/w) of an intragranular lubricant (e.g., magnesium stearate);
(f) about 0.25% (w/w) to about 0.75% (w/w) of an extragranular glidant (e.g., colloidal silicon dioxide);
(g) about 1% (w/w) to about 3% (w/w) of an extragranular disintegrant (e.g., croscarmellose sodium); and
(h) about 0.25% (w/w) to about 0.75% (w/w) of an extragranular lubricant (e.g., magnesium stearate);
thereby totaling no more than 100% (w/w) of the composition.

### Embodiment 117

A pharmaceutical composition of embodiment 114 or 115, wherein the composition comprises:
(a) about 5% (w/w) to about 15% (w/w) of the compound of formula (I) (e.g., crystalline form A);
(b) about 77% (w/w) to about 85% (w/w) of an intragranular filler (e.g., microcrystalline cellulose);
(c) about 0.9% (w/w) to about 1.1% (w/w) of an intragranular glidant (e.g., colloidal silicon dioxide);
(d) about 1.8% (w/w) to about 2.2% (w/w) of an intragranular disintegrant (e.g., croscarmellose sodium);
(e) about 0.9% (w/w) to about 1.1% (w/w) of an intragranular lubricant (e.g., magnesium stearate);
(f) about 0.4% (w/w) to about 0.6% (w/w) of an extragranular glidant (e.g., colloidal silicon dioxide);
(g) about 1.8% (w/w) to about 2.2% (w/w) of an extragranular disintegrant (e.g., croscarmellose sodium); and
(h) about 0.4% (w/w) to about 0.6% (w/w) of an extragranular lubricant (e.g., magnesium stearate);
thereby totaling no more than 100% (w/w) of the composition.

### Embodiment 118

A pharmaceutical composition of embodiment 114 or 115, wherein the composition comprises:
(a) about 8% (w/w) to about 12% (w/w) of the compound of formula (I) (e.g., crystalline form A);
(b) about 80% (w/w) to about 85% (w/w) of an intragranular filler (e.g., microcrystalline cellulose);
(c) about 0.95% (w/w) to about 1.05% (w/w) of an intragranular glidant (e.g., colloidal silicon dioxide);
(d) about 1.9% (w/w) to about 2.1% (w/w) of an intragranular disintegrant (e.g., croscarmellose sodium);
(e) about 0.95% (w/w) to about 1.05% (w/w) of an intragranular lubricant (e.g., magnesium stearate);
(f) about 0.45% (w/w) to about 0.55% (w/w) of an extragranular glidant (e.g., colloidal silicon dioxide);
(g) about 1.9% (w/w) to about 2.1% (w/w) of an extragranular disintegrant (e.g., croscarmellose sodium); and
(h) about 0.45% (w/w) to about 0.55% (w/w) of an extragranular lubricant (e.g., magnesium stearate);
thereby totaling no more than 100% (w/w) of the composition.

### Embodiment 119

A pharmaceutical composition of embodiment 114 or 115, wherein the composition comprises:
(a) about 10.0% (w/w) of the compound of formula (I) (e.g., crystalline form A);
(b) about 83.5% (w/w) of an intragranular filler (e.g., microcrystalline cellulose);
(c) about 1.0% (w/w) of an intragranular glidant (e.g., colloidal silicon dioxide);
(d) about 2% (w/w) of an intragranular disintegrant (e.g., croscarmellose sodium);
(e) about 0.5% (w/w) of an intragranular lubricant (e.g., magnesium stearate);
(f) about 0.5% (w/w) of an extragranular glidant (e.g., colloidal silicon dioxide);
(g) about 2% (w/w) of an extragranular disintegrant (e.g., croscarmellose sodium); and
(h) about 0.5% (w/w) of an extragranular lubricant (e.g., magnesium stearate); thereby totaling no more than 100% (w/w) of the composition.

### Embodiment 120

A pharmaceutical composition of embodiment 114 or 115, wherein the composition comprises:
(a) about 27% (w/w) to about 33% (w/w) of the compound of formula (I);
(b) about 37% (w/w) to about 43% (w/w) of an intragranular filler (e.g., microcrystalline cellulose);
(c) about 0.85% (w/w) to about 0.9% (w/w) of an intragranular glidant (e.g., colloidal silicon dioxide);
(d) about 1.4% (w/w) to about 1.6% (w/w) of an intragranular disintegrant (e.g., croscarmellose sodium);
(e) about 0.85% (w/w) to about 0.9% (w/w) of an intragranular lubricant (e.g., magnesium stearate);
(f) about 0.45% (w/w) to about 0.55% (w/w) of an extragranular glidant (e.g., colloidal silicon dioxide);
(g) about 1.8% (w/w) to about 2.2% (w/w) of an extragranular disintegrant (e.g., croscarmellose sodium); and
(h) about 0.45% (w/w) to about 0.55% (w/w) of an extragranular lubricant (e.g., magnesium stearate);
thereby totaling no more than 100% (w/w) of the composition.

### Embodiment 121

The pharmaceutical composition of any one of embodiments 5 to 120 wherein the intragranular filler is microcrystalline cellulose.

### Embodiment 122

The pharmaceutical composition of any one of embodiments 5 to 120 wherein the intragranular filler is microcrystalline cellulose PH 102.

### Embodiment 123

The pharmaceutical composition of any one of embodiments 5 to 122 wherein the intragranular glidant is colloidal silicon dioxide.

### Embodiment 124

The pharmaceutical composition of any one of embodiments 5 to 123 wherein the extragranular glidant is colloidal silicon dioxide.

### Embodiment 125

The pharmaceutical composition of any one of embodiments 5 to 124 wherein the intragranular disintegrant is croscarmellose sodium.

### Embodiment 126

The pharmaceutical composition of any one of embodiments 5 to 125 wherein the extragranular disintegrant is croscarmellose sodium.

### Embodiment 127

The pharmaceutical composition of any one of embodiments 5 to 126 wherein the intragranular lubricant is magnesium stearate.

### Embodiment 128

The pharmaceutical composition of any one of embodiments 5 to 127 wherein the extragranular lubricant is magnesium stearate.

### Embodiment 129

A dosage form comprising a pharmaceutical composition of any one of embodiments 5 to 128.

### Embodiment 130

The dosage form of embodiment 129, wherein the total weight of the pharmaceutical composition in the dosage form is about 50 mg to 1000 mg.

### Embodiment 131

The dosage form of embodiment 129, wherein the total weight of the pharmaceutical composition in the dosage form is about 100 mg to 750 mg.

### Embodiment 132

The dosage form of embodiment 129, wherein the total weight of the pharmaceutical composition in the dosage form is about 50 mg to 150 mg.

### Embodiment 133

The dosage form of embodiment 129, wherein the total weight of the pharmaceutical composition in the dosage form is about 50 mg, about 60 mg, about 70 mg, about 80 mg, about 90 mg, about 100 mg, about 110 mg, about 120 mg, about 130 mg, about 140 mg or about 150 mg.

### Embodiment 134

The dosage form of embodiment 129, wherein the total weight of the pharmaceutical composition in the dosage form is about 80 mg, about 90 mg, about 100 mg, about 110 mg, or about 120 mg.

### Embodiment 135

The dosage form of embodiment 129, wherein the total weight of the pharmaceutical composition in the dosage form is about 100 mg.

### Embodiment 136

The dosage form of embodiment 129, wherein the total weight of the pharmaceutical composition in the dosage form is about 400 mg to 600.

### Embodiment 137

The dosage form of embodiment 129, wherein the total weight of the pharmaceutical composition in the dosage form is about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg.

### Embodiment 138

The dosage form of embodiment 129, wherein the total weight of the pharmaceutical composition in the dosage form is about 450 mg, about 460 mg, about 480 mg, about 500 mg, about 520 mg, about 540 mg, or about 560 mg.

### Embodiment 139

The dosage form of embodiment 129, wherein the total weight of the pharmaceutical composition in the dosage form is about 500 mg.

### Embodiment 140

The dosage form of embodiment 129, wherein the total weight of the pharmaceutical composition in the dosage form is about 100 mg or about 500 mg.

### Embodiment 141

The dosage form of any one of embodiments 129 to 140, wherein the composition comprises about 5 mg to about 200 mg of a compound of formula (I).

### Embodiment 142

The dosage form of any one of embodiments 129 to 140, wherein the composition comprises about 50 mg to about 100 mg of the compound of formula (I).

### Embodiment 143

The dosage form of any one of embodiments 129 to 140, wherein the composition comprises about 5 mg to about 20 mg, about 20 mg to about 40 mg, about 40 mg to about 60 mg, about 60 mg to about 80 mg or about 80 to about 100 mg of the compound of formula (I).

### Embodiment 144

The dosage form of any one of embodiments 129 to 140, wherein the composition comprises about 5 mg to about 20 mg or about 40 mg to about 60 mg of the compound of formula (I).

### Embodiment 145

The dosage form of any one of embodiments 129 to 140, wherein the composition comprises about 5 mg to about 15 mg of the compound of formula (I).

### Embodiment 146

The dosage form of any one of embodiments 129 to 140, wherein the composition comprises about 8 mg to about 12 mg of the compound of formula (I).

### Embodiment 147

The dosage form of any one of embodiments 129 to 140, wherein the composition comprises about 5 mg, about 6 mg, about 7 mg, 8 mg, about 9 mg, about 10 mg, about 11 mg, about 12 mg, about 13 mg, about 14 mg or about 15 mg of the compound of formula (I).

### Embodiment 148

The dosage form of any one of embodiments 129 to 140, wherein the composition comprises about 10 mg of the compound of formula (I).

### Embodiment 149

The dosage form of any one of embodiments 129 to 140, wherein the composition comprises about 40 mg to about 60 mg of the compound of formula (I).

### Embodiment 150

The dosage form of any one of embodiments 129 to 140, wherein the composition comprises about 45 mg to about 55 mg of the compound of formula (I).

### Embodiment 151

The dosage form of any one of embodiments 129 to 140, wherein the composition comprises about 47 mg to about 53 mg of the compound of formula (I).

### Embodiment 152

The dosage form of any one of embodiments 129 to 140, wherein the composition comprises about 49 mg to about 51 mg of the compound of formula (I).

### Embodiment 153

The dosage form of any one of embodiments 129 to 140, wherein the composition comprises about 45 mg, about 46 mg, about 47 mg, about 48 mg, about 49 mg, about 50 mg, about 51 mg, about 52 mg, about 53 mg, about 54 mg or about 55 mg of the compound of formula (I).

### Embodiment 154

The dosage form of any one of embodiments 129 to 140, wherein the composition comprises about 50 mg of the compound of formula (I).

### Embodiment 155

The dosage form of any one of embodiments 129 to 140, wherein the composition comprises about 5 mg, about 6 mg, about 7 mg, about 8 mg, about 9 mg, about 10 mg, about 11 mg, about 12 mg, about 13 mg, about 14 mg, about 15 mg, about 45 mg, about 46 mg, about 47 mg, about 48 mg, about 49 mg, about 50 mg, about 51 mg, about 52 mg, about 53 mg, about 54 mg or about 55 mg of the compound of formula (I).

### Embodiment 156

The dosage form of any one of embodiments 129 to 140, wherein the composition comprises 8 mg, about 9 mg, about 10 mg, about 11 mg, about 12 mg, about 48 mg, about 49 mg, about 50 mg, about 51 mg or about 52 mg of the compound of formula (I).

### Embodiment 157

The dosage form of any one of embodiments 129 to 140, wherein the composition comprises about 10 mg or about 50 mg of the compound of formula (I).

### Embodiment 158

The dosage form of any one of embodiments 129 to 140 wherein the composition comprises 10 mg Compound of formula (I), 83.5 mg intragranular microcrystalline cellulose, 1 mg intragranular colloidal silicon dioxide, 2 mg intragranular croscarmellose sodium, 0.5 mg intragranular magnesium stearate, 0.5 mg extragranular colloidal silicon dioxide, 2 mg extragranular croscarmellose sodium and 0.5 mg extragranular magnesium stearate.

### Embodiment 159

The dosage form of any one of embodiments 129 to 140 wherein the composition comprises 50 mg Compound of formula (I), 417.5 mg intragranular microcrystalline cellulose, 5 mg intragranular colloidal silicon dioxide, 10 mg intragranular croscarmellose sodium, 2.5 mg intragranular magnesium stearate, 2.5 mg extragranular colloidal silicon dioxide, 10 mg extragranular croscarmellose sodium and 2.5 mg extragranular magnesium stearate.

### Embodiment 160

The dosage form of any one of embodiments 129 to 159, wherein the dosage form is a solid dosage form.

### Embodiment 161

The dosage form of embodiment 129 to 160, wherein the dosage form is an oral dosage form.

### Embodiment 162

The dosage form of any one of embodiments 129 to 161 wherein the dosage form is selected from the group consisting of a powder, a sachet, a stick pack, a capsule, a minitab, and a tablet.

### Embodiment 163

The dosage form of any one of embodiments 129 to 161 wherein the dosage form is a tablet.

### Embodiment 164

The dosage form of embodiment 163, wherein the tablet comprises a coating.

### Embodiment 165

The dosage form of embodiment 164 wherein the coating comprises a polyvinyl alcohol.

### Embodiment 166

A method for treating an MTAP-deficient and/or an MTA-accumulating disease in a subject in need thereof comprising administering to the subject a therapeutically effective amount of a crystalline form of any one of embodiments 1 to 4.

### Embodiment 167

A method for treating an MTAP-deficient and/or an MTA-accumulating disease in a subject in need thereof comprising administering to the subject a pharmaceutical composition of any one of embodiments 5 to 128 containing a therapeutically effective amount of the compound of Formula (I).

### Embodiment 168

A method for treating an MTAP-deficient and/or an MTA-accumulating disease in a subject in need thereof comprising administering to the subject a dosage form of any one of embodiments 129 to 165 containing a therapeutically effective amount of the compound of Formula (I).

### Embodiment 169

The method of any one of embodiments 166 to 168 wherein the disease is a proliferating disease

### Embodiment 170

The method of any one of embodiments 166 to 168 wherein the disease is an MTAP-deficient and/or MTA-accumulating cancer.

### Embodiment 171

A method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering an effective amount (e.g., a therapeutically effective amount) of a crystalline form of any one of embodiments 1 to 4 to the subject identified in step b).

### Embodiment 172

A method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering the pharmaceutical composition of any one of embodiments 5 to 128 containing an effective amount (e.g., a therapeutically effective amount) of the compound of formula a (I) to the subject identified in step b).

### Embodiment 173

A method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering the dosage form of any one of embodiments 129 to 165 containing an effective amount (e.g., a therapeutically effective amount) of the compound of formula (I) to the subject identified in step b).

### Embodiment 174

The method of any one of embodiments 166 to 173 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma, e.g., pancreatic ductal adenocarcinoma (PDAC)), mesothelioma, melanoma, non-small cell lung cancer (NSCLC; e.g., lung squamous or lung adenocarcinoma), astrocytoma, undifferentiated pleomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

### Embodiment 175

The method of any one of embodiments 166 to 173 wherein the cancer is glioblastoma, glioblastoma multiforme, urothelial carcinoma, malignant peripheral nerve sheath tumor (MPNST), cholangiocarcinoma, NSCLC (e.g., squamous and adenocarcinoma), bladder cancer, pancreatic cancer (e.g., pancreatic ductal adenocarcinoma (PDAC)), mesothelioma, colorectal cancer, chronic myelogenous leukemia (CML), or diffuse large B-cell lymphoma (DLBCL).

### Embodiment 176

The method of any one of embodiments 166 to 173 wherein the cancer is a glioblastoma, glioblastoma multiforme, cholangiocarcinoma, NSCLC (e.g., squamous and adenocarcinoma), bladder cancer, pancreatic cancer (e.g., pancreatic ductal adenocarcinoma (PDAC)) mesothelioma, colorectal cancer, chronic myelogenous leukemia (CML), or diffuse large B-cell lymphoma (DLBCL).

### Embodiment 177

The method of any one of embodiments 166 to 173 wherein the cancer is cholangiocarcinoma, NSCLC (squamous), NSCLC (adenocarcinoma), bladder cancer, pancreatic cancer (e.g., pancreatic ductal adenocarcinoma (PDAC)) mesothelioma, colorectal cancer, chronic myelogenous leukemia (CML), or diffuse large B-cell lymphoma (DLBCL).

### Embodiment 178

The method of any one of embodiments 166 to 173 wherein the cancer is glioma, glioblastoma, non-small cell lung cancer (adenocarcinoma and squamous), mesothelioma, cholangiocarcinoma, bladder cancer, urothelial carcinoma, pancreatic cancer (e.g., pancreatic ductal adenocarcinoma (PDAC)) or malignant peripheral nerve sheath tumor.

### Embodiment 179

The method of any one of embodiments 166 to 173 wherein the cancer is cholangiocarcinoma, NSCLC (squamous), NSCLC (adenocarcinoma), bladder cancer, pancreatic cancer (e.g., pancreatic ductal adenocarcinoma (PDAC)) or mesothelioma.

### Embodiment 180

The method of any one of embodiments 166 to 173 wherein the cancer is cholangiocarcinoma, NSCLC (squamous), bladder cancer, pancreatic cancer (e.g., pancreatic ductal adenocarcinoma (PDAC)) or mesothelioma.

### Embodiment 181

The method of any one of embodiments 166 to 173 wherein the cancer is a CNS tumor (e.g., glioma, glioblastoma).

### Embodiment 182

The method of embodiment 181 wherein the CNS tumor is glioma.

### Embodiment 183

The method of embodiment 181 wherein the CNS tumor is glioblastoma.

### Embodiment 184

The method of embodiment 181 wherein the CNS tumor is glioblastoma multiforme (GBM).

### Embodiment 185

The method of any one of embodiments 166 to 173 wherein the cancer is not a CNS tumor.

### Embodiment 186

The method of any one of embodiments 166 to 173 wherein the cancer is leukemia (e.g., chronic myelogenous leukemia).

### Embodiment 187

The method of any one of embodiments 166 to 173 wherein the cancer is colorectal cancer.

### Embodiment 188

The method of any one of embodiments 166 to 173 wherein the cancer is non-small cell lung cancer (e.g., lung squamous or lung adenocarcinoma).

### Embodiment 189

The method of any one of embodiments 166 to 173 wherein the cancer is pancreatic cancer (e.g., pancreatic ductal adenocarcinoma).

### Embodiment 190

The method of any one of embodiments 166 to 173 wherein the cancer is bladder cancer (e.g., bladder urothelial carcinoma).

### Embodiment 191

The method of any one of embodiments 166 to 173 wherein the cancer is cholangiocarcinoma.

### Embodiment 192

The method of any one of embodiments 166 to 173 wherein the cancer is mesothelioma.

### Embodiment 193

The method of any one of embodiments 166 to 173 wherein the cancer is diffuse large B cell lymphoma (DLBCL).

### Embodiment 194

The method of any one of embodiments 166 to 173 wherein the cancer is a malignant peripheral nerve sheath tumor (MPNST).

### Embodiment 195

The method of any one of embodiments 166 to 194, wherein the method comprises administering to the subject a dose of about 5 mg to about 1000 mg of the compound of Formula (I) once or twice daily.

### Embodiment 196

The method of any one of embodiments 166 to 194, wherein the method comprises administering to the subject a dose of about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 200 mg, about 10 mg to about 150 mg, about 10 mg to about 120 mg, about 10 mg to about 100 mg, about 10 mg to about 50 mg, about 10 mg to about 20 mg, about 20 mg to about 500 mg, about 20 mg to about 450 mg, about 20 mg to about 400 mg, about 20 mg to about 350 mg, about 20 mg to about 320 mg, about 20 mg to about 300 mg, about 20 mg to about 250 mg, about 20 mg to about 200 mg, about 20 mg to about 150 mg, about 20 mg to about 120 mg, about 20 mg to about 100 mg, about 20 mg to about 50 mg, about 40 mg to about 500 mg, about 40 mg to about 450 mg, about 40 mg to about 400 mg, about 40 mg to about 350 mg, about 40 mg to about 320 mg, about 40 mg to about 300 mg, about 40 mg to about 250 mg, about 40 mg to about 200 mg, about 40 mg to about 150 mg, about 40 mg to about 120 mg, about 40 mg to about 100 mg, about 40 mg to about 50 mg, about 50 mg to about 500 mg, about 50 mg to about 450 mg, about 50 mg to about 400 mg, about 50 mg to about 350 mg, about 50 mg to about 320 mg, about 50 mg to about 300 mg, about 50 mg to about 250 mg, about 50 mg to about 200 mg, about 50 mg to about 150 mg, about 50 mg to about 120 mg, about 50 mg to about 100 mg, about 100 mg to about 500 mg, about 100 mg to about 450 mg, about 100 mg to about 400 mg, about 100 mg to about 350 mg, about 100 mg to about 320 mg, about 100 mg to about 300 mg, about 100 mg to about 250 mg, about 100 mg to about 200 mg, about 100 mg to about 150 mg, about 100 mg to about 120 mg, about 120 mg to about 500 mg, about 120 mg to about 450 mg, about 120 mg to about 400 mg, about 120 mg to about 350 mg, about 120 mg to about 320 mg, about 120 mg to about 300 mg, about 120 mg to about 250 mg, about 120 mg to about 200 mg, about 120 mg to about 150 mg, about 150 mg to about 500 mg, about 150 mg to about 450 mg, about 150 mg to about 400 mg, about 150 mg to about 350 mg, about 150 mg to about 320 mg, about 150 mg to about 300 mg, about 150 mg to about 250 mg, about 150 mg to about 200 mg, about 200 mg to about 500 mg, about 200 mg to about 450 mg, about 200 mg to about 400 mg, about 200 mg to about 350 mg, about 200 mg to about 320 mg, about 200 mg to about 300 mg, about 200 mg to about 250 mg, about 250 mg to about 500 mg, about 250 mg to about 450 mg, about 250 mg to about 400 mg, about 250 mg to about 350 mg, about 250 mg to about 320 mg, about 250 mg to about 300 mg, about 300 mg to about 500 mg, about 300 mg to about 450 mg, about 300 mg to about 400 mg, about 300 mg to about 350 mg, about 300 mg to about 320 mg, about 320 mg to about 500 mg, about 320 mg to about 450 mg, about 320 mg to about 400 mg, about 320 mg to about 350 mg, about 350 mg to about 500 mg, about 350 mg to about 450 mg, about 350 mg to about 400 mg, about 400 mg to about 500 mg, about 400 mg to about 450 mg or about 450 mg to about 500 mg of the compound of Formula (I) once or twice daily.

### Embodiment 197

The method of any one of embodiments 166 to 194, wherein the method comprises administering to the subject a dose of about 10 mg to about 500 mg of the compound of Formula (I) once or twice daily.

### Embodiment 198

The method of any one of embodiments 166 to 194, wherein the method comprises administering to the subject a dose of about 20 mg to about 400 mg of the compound of Formula (I) once or twice daily.

### Embodiment 199

The method of any one of embodiments 166 to 194, wherein the method comprises administering to the subject a dose of about 20 mg to about 300 mg of the compound of Formula (I) once or twice daily.

### Embodiment 200

The method of any one of embodiments 166 to 194, wherein the method comprises administering to the subject a dose of about 15 mg to about 25 mg of the compound of Formula (I) once or twice daily.

### Embodiment 201

The method of any one of embodiments 166 to 194, wherein the method comprises administering to the subject a dose of about 18 mg to about 22 mg of the compound of Formula (I) once or twice daily.

### Embodiment 202

The method of any one of embodiments 166 to 194, wherein the method comprises administering to the subject a dose of about 5 mg, about 6 mg, about 7 mg, 8 mg, about 9 mg, about 10 mg, about 11 mg, about 12 mg, about 13 mg, about 14 mg, or about 15 mg of the compound of Formula (I) once or twice daily.

### Embodiment 203

The method of any one of embodiments 166 to 194, wherein the method comprises administering to the subject a dose of about 20 mg of the compound of Formula (I) once or twice daily.

### Embodiment 204

The method of any one of embodiments 166 to 194, wherein the method comprises administering to the subject a dose of about 40 mg to about 60 mg of the compound of Formula (I) once or twice daily.

### Embodiment 205

The method of any one of embodiments 166 to 194, wherein the method comprises administering to the subject a dose of about 45 mg to about 55 mg of the compound of Formula (I) once or twice daily.

### Embodiment 206

The method of any one of embodiments 166 to 194, wherein the method comprises administering to the subject a dose of about 47 mg to about 53 mg of the compound of Formula (I) once or twice daily.

### Embodiment 207

The method of any one of embodiments 166 to 194, wherein the method comprises administering to the subject a dose of about 49 mg to about 51 mg of the compound of Formula (I) once or twice daily.

### Embodiment 208

The method of any one of embodiments 166 to 194, wherein the method comprises administering to the subject a dose of about 45 mg, about 46 mg, about 47 mg, about 48 mg, about 49 mg, about 50 mg, about 51 mg, about 52 mg, about 53 mg, about 54 mg or about 55 mg of the compound of Formula (I) once or twice daily.

### Embodiment 209

The method of any one of embodiments 166 to 194, wherein the method comprises administering to the subject a dose of about 50 mg of the compound of Formula (I) once or twice daily.

### Embodiment 210

The method of any one of embodiments 166 to 194, wherein the method comprises administering to the subject a dose of about 15 mg, about 16 mg, about 17 mg, 18 mg, about 19 mg, about 20 mg, about 21 mg, about 22 mg, about 23 mg, about 24 mg, about 25 mg, about 45 mg, about 46 mg, about 47 mg, about 48 mg, about 49 mg, about 50 mg, about 51 mg, about 52 mg, about 53 mg, about 54 mg or about 55 mg of the compound of Formula (I) once or twice daily.

### Embodiment 211

The method of any one of embodiments 166 to 194, wherein the method comprises administering to the subject a dose of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg or about 500 mg of the compound of Formula (I) once or twice daily.

### Embodiment 212

The method of any one of embodiments 166 to 194, wherein the method comprises administering to the subject a dose of about 10 mg, about 20 mg, about 50 mg, about 60 mg, about 100 mg, about 120 mg, about 150 mg, about 200 mg, about 250 mg, about 300 mg, about 320 mg, about 350 mg, about 400 mg, about 450 mg or about 500 mg of the compound of Formula (I) once or twice daily.

### Embodiment 213

The method of any one of embodiments 166 to 194, wherein the method comprises administering to the subject a dose of about 20 mg, about 40 mg, about 50 mg, about 60 mg, about 80 mg, about 100 mg, about 120 mg, about 150 mg, about 200 mg, about 250 mg, about 300 mg, about 320 mg, about 350 mg, about 400 mg, about 450 mg or about 500 mg of the compound of Formula (I) once or twice daily.

### Embodiment 214

The method of any one of embodiments 166 to 194, wherein the method comprises administering to the subject a dose of about 20 mg or about 50 mg of the compound of Formula (I) once or twice daily.

### Embodiment 215

The method of any one of embodiments 166 to 214, wherein the method comprises administering the dose of the compound of Formula (I) once daily.

### Embodiment 216

The method of any one of embodiments 166 to 214, wherein the method comprises administering the dose of the compound of Formula (I) twice daily.

### Embodiment 217

The method of any one of embodiments 166 to 216, wherein the method further comprises administration of a second therapeutic agent.

### EXAMPLES

In order that the invention described herein may be more fully understood, the following examples are set forth. The examples described in this application are offered to illustrate the crystalline solid forms provided herein and are not to be construed in any way as limiting their scope.

### Abbreviations and Definitions

API active pharmaceutical ingredient
AUC area under the curve
BA bioavailability
Cₘₐₓ maximum observed plasma concentration
DMA N,N-dimethylacetamide
DP drug product
DS drug substance
DSC differential scanning calorimetry
dm/dt change in mass per unit of time
DVS dynamic vapor sorption
FaSSIF fasted state simulated intestinal fluid
FeSSIF fed state simulated intestinal fluid
GMP Good Manufacturing Practice
HPLC high performance liquid chromatography
LOD limit of detection
max maximum
min minimum
MCC microcrystalline cellulose
NA not applicable
RSD relative standard deviation
RH relative humidity
RT room temperature or real time
sccm standard cubic centimeter per minute
SD Standard Deviation
TGA thermogravimetric analysis
USP United States Pharmacopoeia
w/w weight-to-weight
XRPD X-ray powder diffraction

### Example 1. Exemplary Preparation of Crystalline Form A

Slow evaporation at about room temperature of a solution obtained from about 20 mg Compound (I) dissolved in 1.0 mL acetone and filtered by centrifugation resulted in the formation of crystalline Form A.

### Example 2. Additional Exemplary Preparation of Crystalline Form A

A sample of Compound of formula (I) was dissolved in acetone at 50° C. The solution was filtered by centrifugation, then cooled to 0° C. in an ice bath and agitated. The precipitate was collected by centrifugation filtration and determined to correspond to crystalline form A.

### Example 3. Exemplary Thermogravimetric Characterization of Crystalline Form A

TG-FTIR: Thermogravimetric measurements were carried out with a Discovery 5500 or Q5000 instrument coupled to a Bruker FTIR Spectrometer Vector 22 (aluminum open sample pan, N2 atmosphere, heating rate 10° C./min). The temperature range was ambient to 300° C. or abort next segment if weight <80% (w/w). The TG-FTIR spectrum of Compound I is shown in FIG. 2A.

### Example 4. Exemplary Differential Scanning Calorimetry (DSC) Characterization of Crystalline Form A

Measurements were carried out with a TA Discovery 2500 or Q2000 instrument (sample pan Tzero pan and Tzero hermetic lid with a pin hole of 0.7 mm in diameter), under a nitrogen flow of 50 mL/min, with a heating rate of 10° C./min. A temperature range of 30-250° C. was scanned before decomposition. The DSC spectrum of Compound I is shown in FIG. 2B.

### Example 5. XRPD Measurements for Crystalline Form A

X-ray powder diffraction was carried out as a two theta theta coupled measurement, using a Bruker D8 Advance XRPD diffractometer equipped with a LYNXEYE (1D mode) detector (with a PSD opening of 2.1°), operating with Cu-Kα1 radiation. The measurements with this instrument were performed at a tube voltage of 40 kV and tube current of 40 mA. The following parameters were set for the coupled 2θ/θ scan: continuous PSD fast mode; 0.02° 2θ step size; 0.12 s or 0.3 s step time; 3-40° or 2-40° 20 scanning range. An appropriate amount of sample (e.g., 20-50 mg) was placed in the central area of a monocrystalline silicone wafer. If necessary, a thin layer of petroleum jelly or silicone oil was applied to the surface of the single silicon wafer to attach the sample, and the excess sample was removed by gentle tapping. The sample plate was loaded into the XRPD sample holder. The sample was rotated during the measurement. All sample preparation and measurement were done in an ambient air atmosphere. The XRPD pattern obtained for crystalline form A is shown in FIG. 1.

### Example 6. Exemplary Dynamic Vapor Sorption Measurements

Dynamic vapor sorption: DVS measurements were performed with an SMS Dynamic Vapor Sorption Intrinsic, Advantage or Adventure System with a total gas flow of 200 sccm, at an oven temperature of 25° C. Humidity changes were performed in steps of 10%, in a sequence of 40-0-95-0-40% humidity, with equilibrium being 0.002 dm/dt (%/min), minimum dm/dt stability duration of 60 minutes and maximum dm/dt stability duration of 360 minutes. The results from the DVS test are presented in FIG. 3A and FIG. 3B, showing an uptake of −0.2% water on the sorption curve between 40% RH and 80% RH, at 25° C. Based on these results, the material is nonhygroscopic below 80% RH at 25° C.

### Example 7. Exemplary Pharmaceutical Compositions and Dosage Forms

The pharmaceutical compositions and dosage forms examples described in this application are offered to illustrate embodiments of the compounds, pharmaceutical compositions, and methods provided herein and are not to be construed in any way as limiting their scope. For example, an excipient may act with a one or more similar or various functions (e.g., filler, glidant, disintegrant, etc.) and identification of a particular function herein is not to be construed in any way as limiting the scope of the respective component.

| | | | Dosage Strength, mg | | |
|---|---|---|---|---|---|
| | | | | 10 | 50 |
| | | | 10/50 | Unit | Unit |
| | | | Formulation | Weight, | Weight, |
| Component | | Function | Composition | mg | mg |
| Intragranular | Compound of | API | 10.0% | 10.0 | 50.0 |
| | formula (I) | | | | |
| | microcrystalline | Filler | 83.5% | 83.5 | 417.5 |
| | cellulose PH 102 | | | | |
| | (Avicel ® PH102) | | | | |
| | Colloidal Silicon | Glidant | 1.0% | 1.0 | 5.0 |
| | Dioxide (CAB-O- | | | | |
| | SIL ® M-5P) | | | | |
| | croscarmellose | Disintegrant | 2.0% | 2.0 | 10.0 |
| | sodium (Ac-Di-Sol ®) | | | | |
| | Magnesium Stearate | Lubricant | 0.5% | 0.5 | 2.5 |
| Extragranular | Colloidal Silicon | Glidant | 0.5% | 0.5 | 2.5 |
| | Dioxide (CAB-O- | | | | |
| | SIL ® M-5P) | | | | |
| | croscarmellose | Disintegrant | 2.0% | 2.0 | 10.0 |
| | sodium (Ac-Di-Sol ®) | | | | |
| | Magnesium Stearate | Lubricant | 0.5% | 0.5 | 2.5 |
| Total Tablet Weight | | | 100.0% | 100.0 | 500.0 |
| Coating | Opadry ® II White | Coating | 3.5% of Core | 3.5 | 17.5 |
| | 85F18422 | | | | |

### Example 8. The Antiproliferative Properties of the Compound of Formula (I)

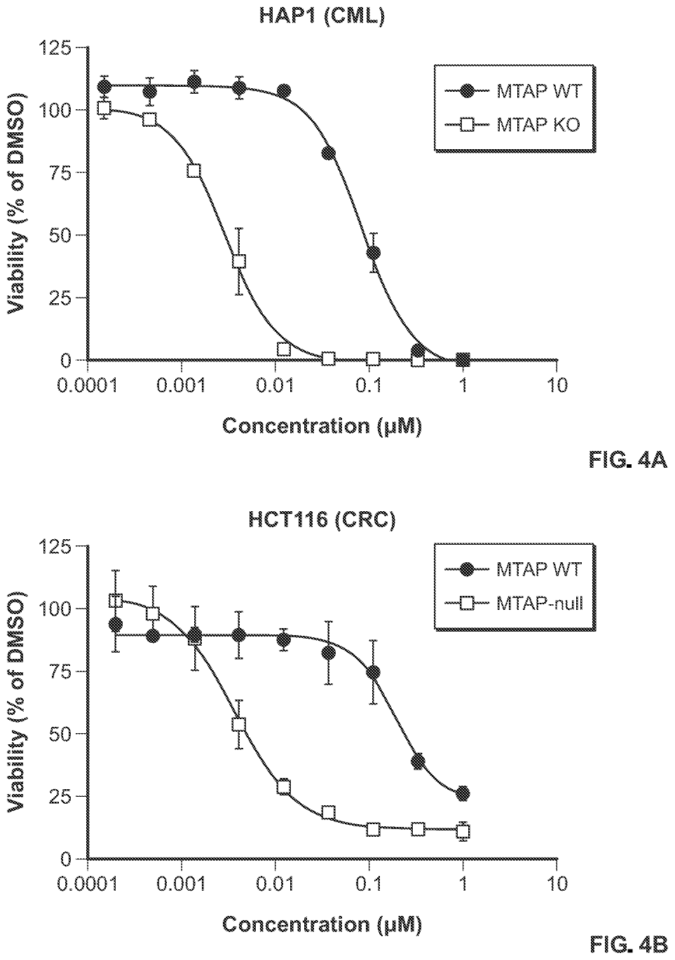

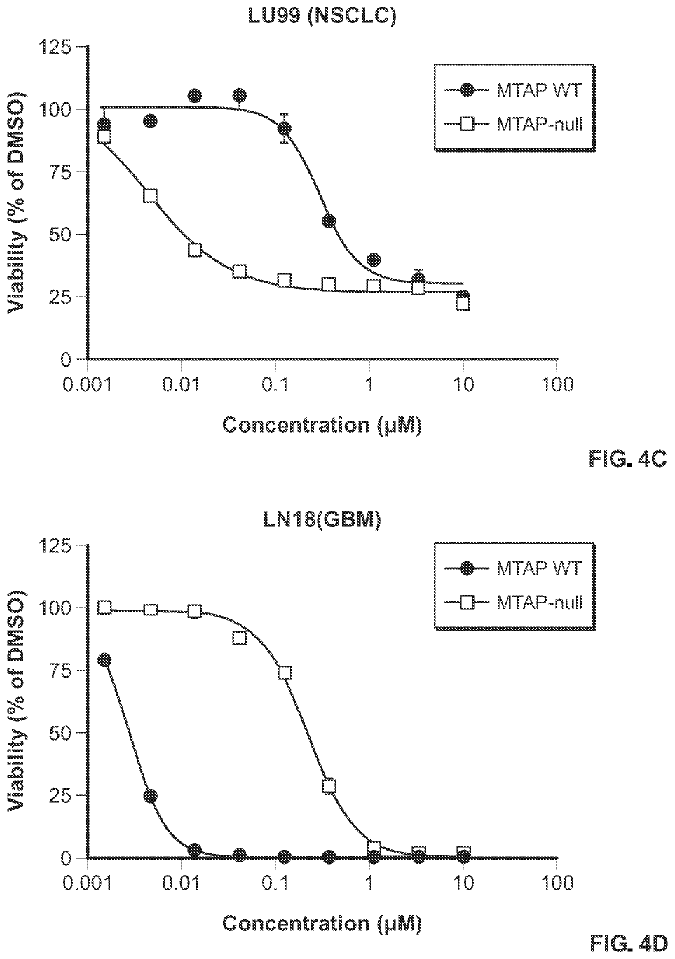

MTAP-null-selectivity of the PRMT5 inhibitor of Formula (I) was determined in a cell based assay in vitro. Briefly, MTAP-isogenic cell lines were engineered by either CRISPR-mediated MTAP gene knockout (HAP1 and HCT116) or by reconstituting exogenous MTAP in an endogenous MTAP-deleted cell line (LU99 and LN18). Cells were treated with various amounts of PRMT5 inhibitor and live cells were determined at day 7 with the CellTiter-Glo viability assay. The data with each of the four cancer cell lines are included in FIG. 4A to FIG. 4D.

### Example 9. The Biochemical Selectivity of the Compound of Formula (I)

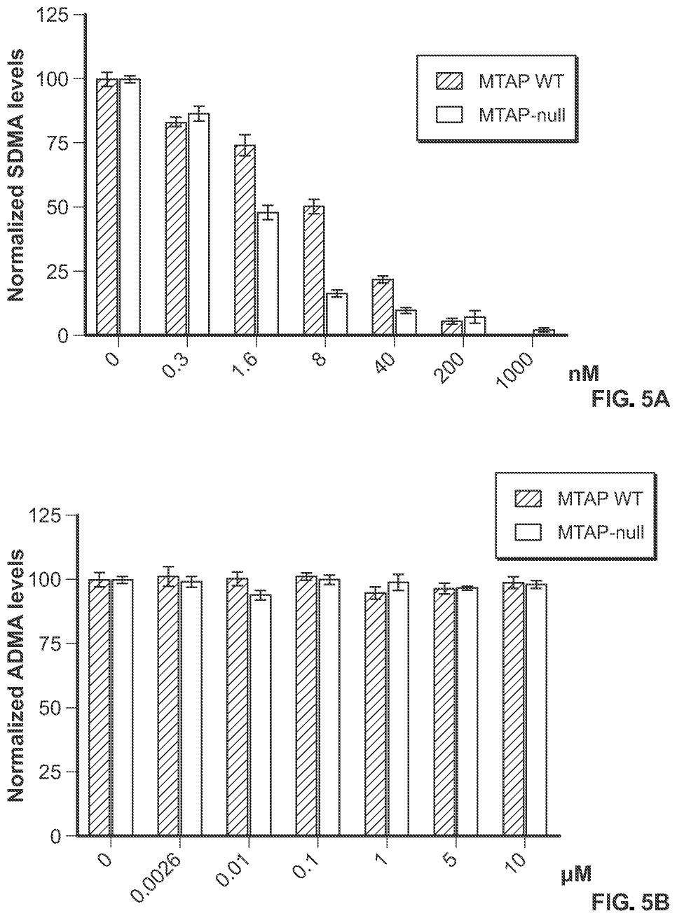

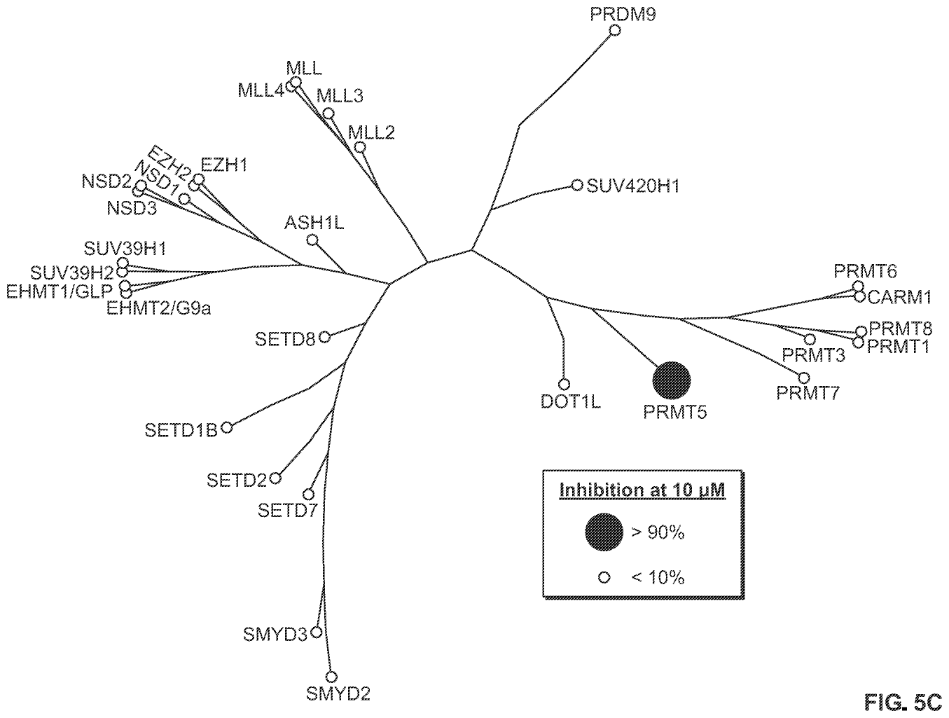

The pharmacodynamic activity of PRMT5 inhibitor of Formula (I) to inhibit PRMT5 in a HAP1 MTAP-isogenic cell line pair was determined. Symmetric dimethylarginine (SDMA) levels at several concentrations of PRMT5 inhibitor of Formula (I) (0, 0.3, 1.6, 8, 40, 200 and 1000 nM) were determined in MTAP WT and MTAP-null HAP1 cell lines. The results show that the PRMT5 inhibitor of Formula (I) selectively inhibits PRMT5 (FIG. 5A) in a dose dependent manner in vitro.

The pharmacodynamic activity of PRMT5 inhibitor of Formula (I) to inhibit Type I PRMTs (including PRMT1) in a HAP1 MTAP-isogenic cell line pair was determined. Asymmetric dimethylarginine (ADMA) levels at several concentrations of PRMT5 inhibitor of Formula (I) (0, 0.0026, 0.01, 0.1, 1, 5 and 10 μm) were determined in MTAP WT and MTAP-null HAP1 cell lines. The results show that the PRMT5 inhibitor of Formula (I) does not inhibit Type I PRMTs (including PRMT1) in either of the cell lines (FIG. 5B).

The biochemical selectivity of the compound of Formula (I) for PRMT5 was tested in a histone methyltransferase panel. The data is shown in FIG. 5C.

### Example 10. Pharmacokinetic Properties of the Compound of Formula (I)

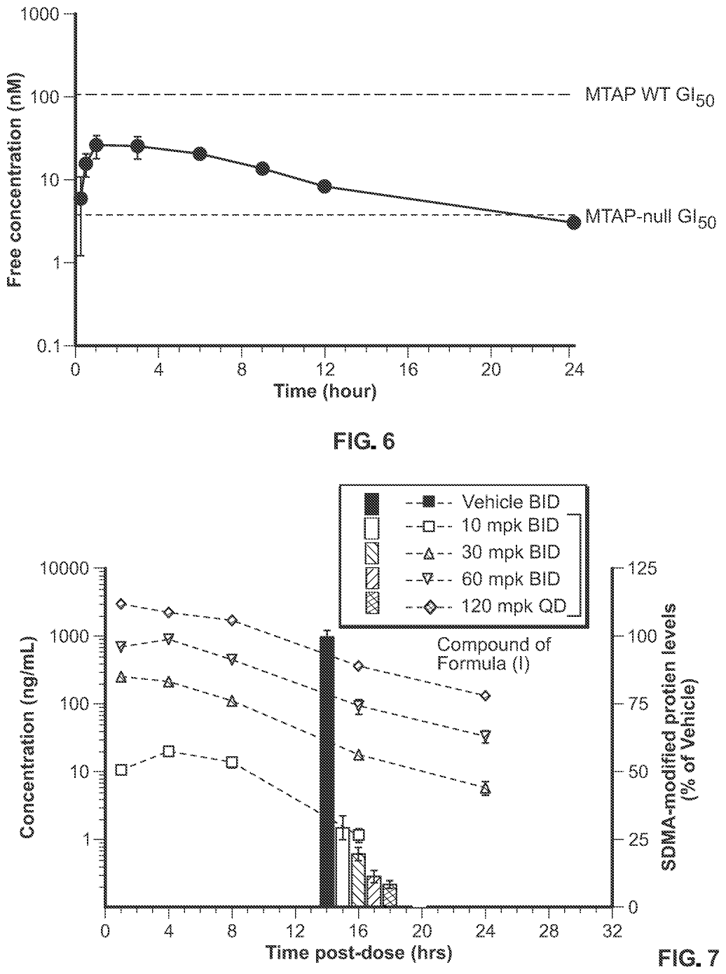

The compound of Formula (I) exhibits good pharmacokinetic properties when dosed orally in cynomolgous monkeys. The compound of formula (I) was administered to the monkeys (n=3) at 3 mg/kg via oral gavage. The data is shown in FIG. 6. The dotted lines indicate the HAP1 MTAP-null and MTAP WT GI₅₀s from a 7-day viability assay the compound. The data is presented as mean±SD.

### Example 11. Screening Cancer Cell Lines with the Compound of Formula (I)

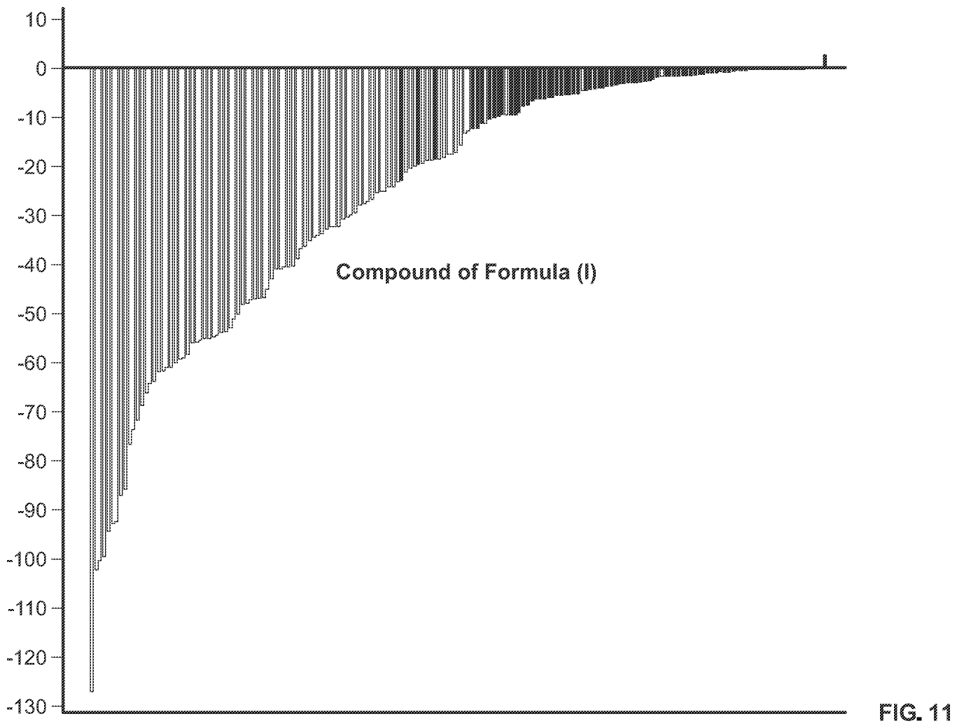

180 cell lines representing multiple cancer lineages (e.g., non-small cell lung cancer, pancreatic ductal adenocarcinoma, bladder cancer, CNS cancers, and heme malignancies) were profiled with the PRMT5 inhibitor of Formula (I) in a 7-day CellTiter-Glo assay. The maximum effect (Amax) at 1 μM (10× the GI₅₀) for each cell line was reported, and the cell lines were fill-coded by MTAP status (MTAP-null in white, MTAP WT in black). The data from the compound of Formula (I) are shown in FIG. 11. MTAP-null cell lines were selectively targeted in the PRMT5 inhibitor of Formula (I).

### Example 12. The Antitumor Activity and the Pharmacokinetic/Pharmacodynamic Model of the Compound of Formula (I) in Xenograft Models

A 7-day pharmacokinetic/pharmacodynamic model of the compound of Formula (I) was conducted using a LU99 (lung giant cell carcinoma) MTAP^{null} xenograft model. The compound of Formula (I) was dosed as indicated (10 mg/kg BID, 30 mg/kg BID, 60 mg/kg BID, 120 mg/kg QD), and the PK and tumor samples were harvested at the timepoints indicated. The SDMA-modified protein levels for each dose as a proportion of vehicle are shown. N=4 tumors per group, and data are presented in FIG. 7 as mean±SEM.

### Example 13. The Dose-Dependent Antitumor Activity of the Compound of Formula (I)

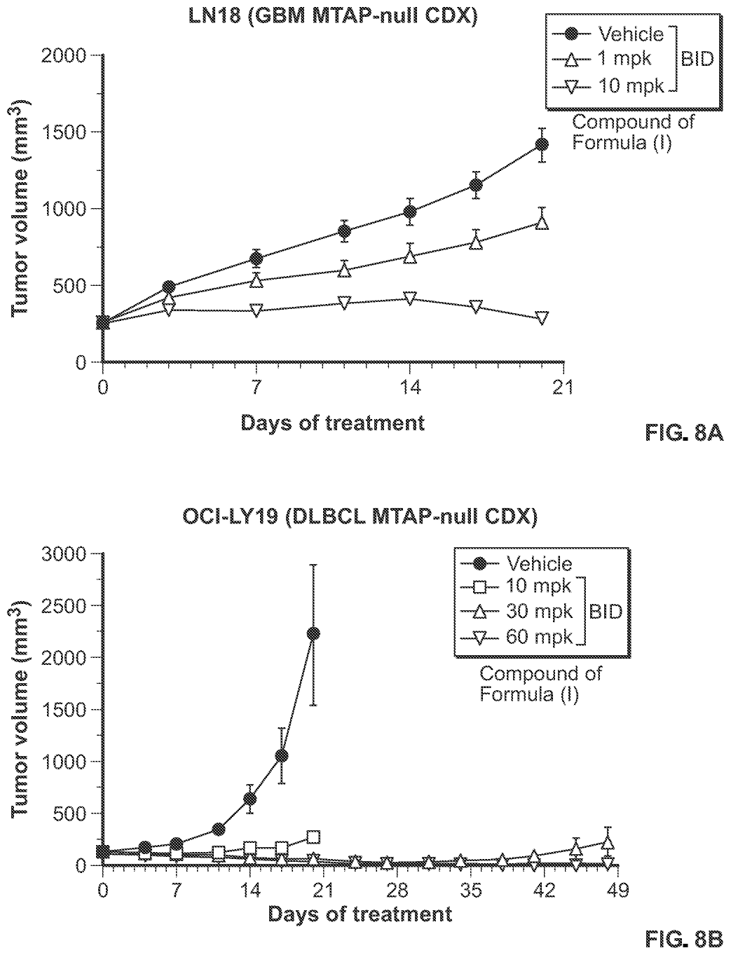

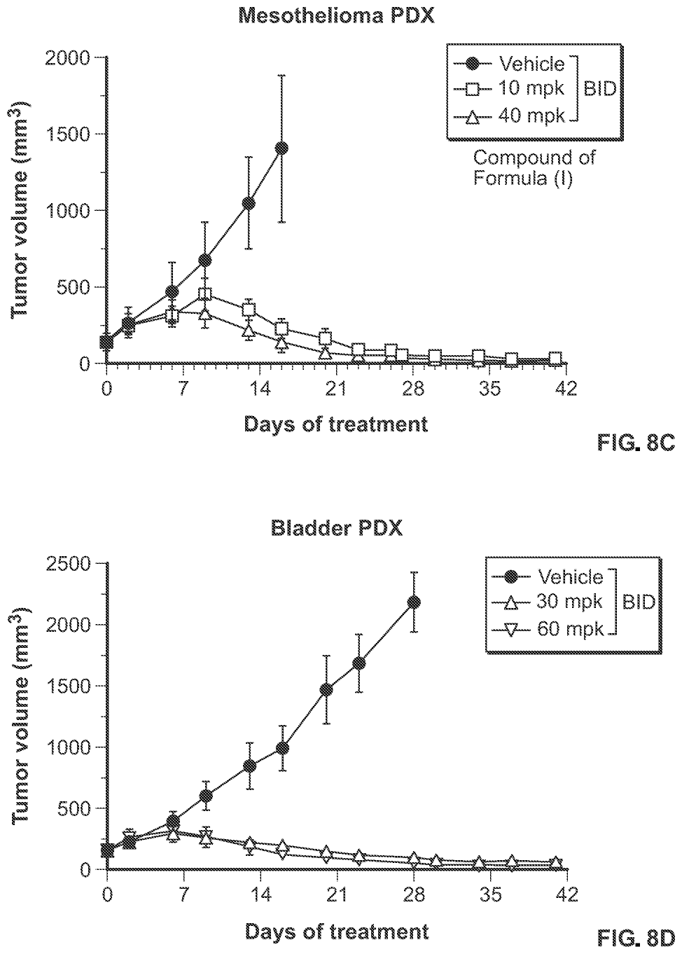

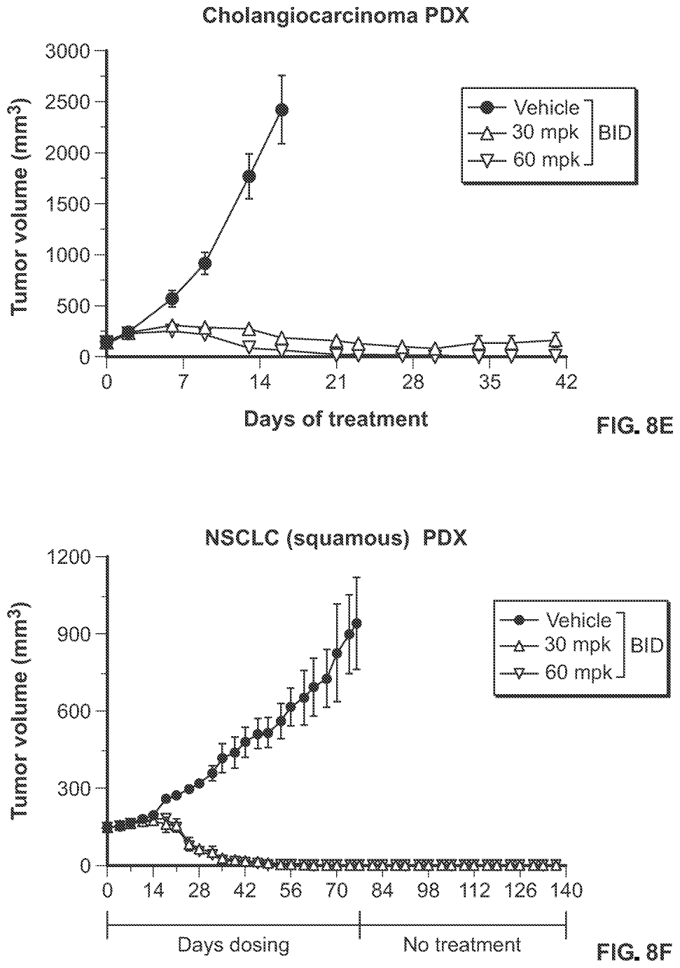

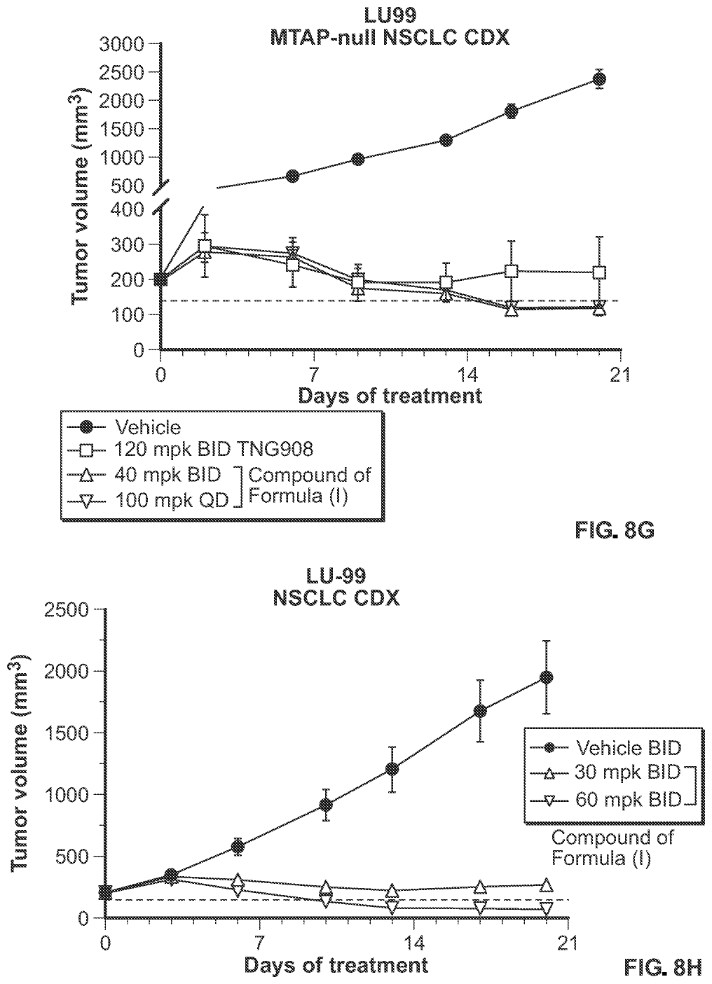

Briefly, the tumor implanted mice (n=3-8 mice per group) were dosed with the indicated doses of PRMT5 inhibitor of Formula (I) or vehicle BID for a minimum of 20 days. Tumor volumes were monitored for the indicated time period, including post-cessation of dosing in the NSCLC model. The results show that PRMT5 inhibitor of Formula (I) was efficacious against the glioblastoma, DLBCL, mesothelioma, bladder, cholangiocarcinoma, and NSCLC tumors (FIGS. 8A-8F).

LN18 MTAP-null cell lines (GBM). The mice were divided into three groups, 8 mice per group as follows: Group 1 was treated with vehicle, group 2 was treated with the compound of Formula (I) 1 mg/kg BID, and group 3 was treated with the compound of Formula (I) 10 mg/kg BID. The data is shown in FIG. 8A.

OCI-LY19 MTAP-null cell lines (DLBCL). The mice were divided into four groups, 8 mice per group as follows: Group 1 was treated with vehicle, group 2 was treated with the compound of Formula (I) 10 mg/kg BID, group 3 was treated with the compound of Formula (I) 30 mg/kg BID and group 4 was treated with the compound of Formula (I) 60 mg/kg BID. The data is shown in FIG. 8B.

Patient derived mesothelioma xenograft. The mice were divided into three groups, 3 mice per group as follows: Group 1 was treated with vehicle, group 2 was treated with the compound of Formula (I) 10 mg/kg BID, and group 3 was treated with the compound of Formula (I) 40 mg/kg BID. The data is shown in FIG. 8C.

Patent derived bladder cancer xenograft. The mice were divided into three groups, 6 mice per group as follows: Group 1 was treated with vehicle, group 2 was treated with the compound of Formula (I) 30 mg/kg BID, and group 3 was treated with the compound of Formula (I) 60 mg/kg BID. The data is shown in FIG. 8D.

Patient derived cholangiocarcinoma xenograft. The mice were divided into three groups, 6 mice per group as follows: Group 1 was treated with vehicle, group 2 was treated with the compound of Formula (I) 30 mg/kg BID, and group 3 was treated with the compound of Formula (I) 60 mg/kg BID. The data is shown in FIG. 8E.

Patient derived NSCLC (squamous) xenograft. The mice were divided into three groups, 3 mice per group as follows: Group 1 was treated with vehicle, group 2 was treated with the compound of Formula (I) 30 mg/kg BID, and group 3 was treated with the compound of Formula (I) 60 mg/kg BID. Tumor volumes were monitored for during dosing and post-cessation of dosing for a total of 140 days. The data is shown in FIG. 8F.

MTAP-null LU99 cell lines (NSCLC). The mice were divided into six groups, 8 mice per group as follows: Group 1 was treated with vehicle, group 2 was treated with TNG908 120 mg/kg BID, group 3 was treated with the compound of Formula (I) 40 mg/kg BID, group 4 was treated with the compound of Formula (I) 100 mg/kg QD, group 5 was treated with the compound of Formula (I) 30 mg/kg BID, and group 6 was treated with the compound of Formula (I) 60 mg/kg BID. The data is shown in FIG. 8G (TNG908 120 mg BID and Compound of formula (I) 40 mg/kg BID and 100 mg/kg QD) and FIG. 8H (Compound of formula (I) 30 mg/kg BID and 60 mg/kg QD).

### Example 14. The Antitumor Activity of the Compound of Formula (I) Against Patient Derived Xenograft Models

The efficacy of PRMT5 inhibitor of Formula (I) was determined in PDX xenograft mouse models across clinically relevant lineages such as bladder cancer, cholangiocarcinoma, mesothelioma, non-small cell lung cancer (e.g., adenocarcinoma and squamous cell carcinoma), and pancreatic cancer (e.g., pancreatic ductal adenocarcinoma). Mice (n=3/group, except one bladder and one cholangiocarcinoma experiment with n=6/group) were dosed with vehicle control or the compound of Formula (I) at 40 mg/kg BID in acidified water or at 60 mg/kg BID in 5% DMA/20% Captisol. The two dosages/formulations produced similar exposures.

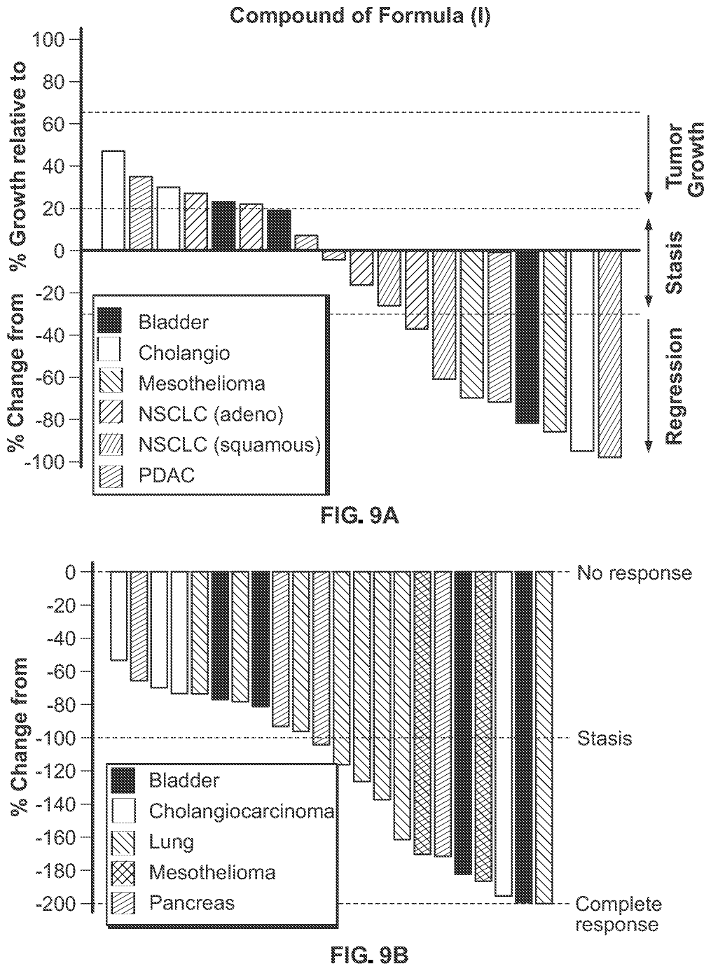

Tumor volume was measured and plotted for % growth relative to vehicle and is shown in the waterfall diagram in FIG. 9A. The results show that PRMT5 inhibitor of Formula (I) drives a strong, lineage agnostic antitumor response in vivo.

Tumor response was calculated for this data set and plotted in the waterfall diagram in FIG. 9B. -% tumor growth inhibition (TGI) shows that these tumors had a Tumor Volume_{final}≥Tumor Volumeᵢₙᵢₜᵢₐₗ (values −100 to 0). % Tumor Volumeᵢₙᵢₜᵢₐₗ−100 is reported for models with Tumor Volume_{final}<Tumor Volumeᵢₙᵢₜᵢₐₗ (values −200 to −100).

### Example 15. The Compound of Formula (I) Resensitizes Tumors with Incomplete Response to an MTA-Cooperative PRMT5 Inhibitor

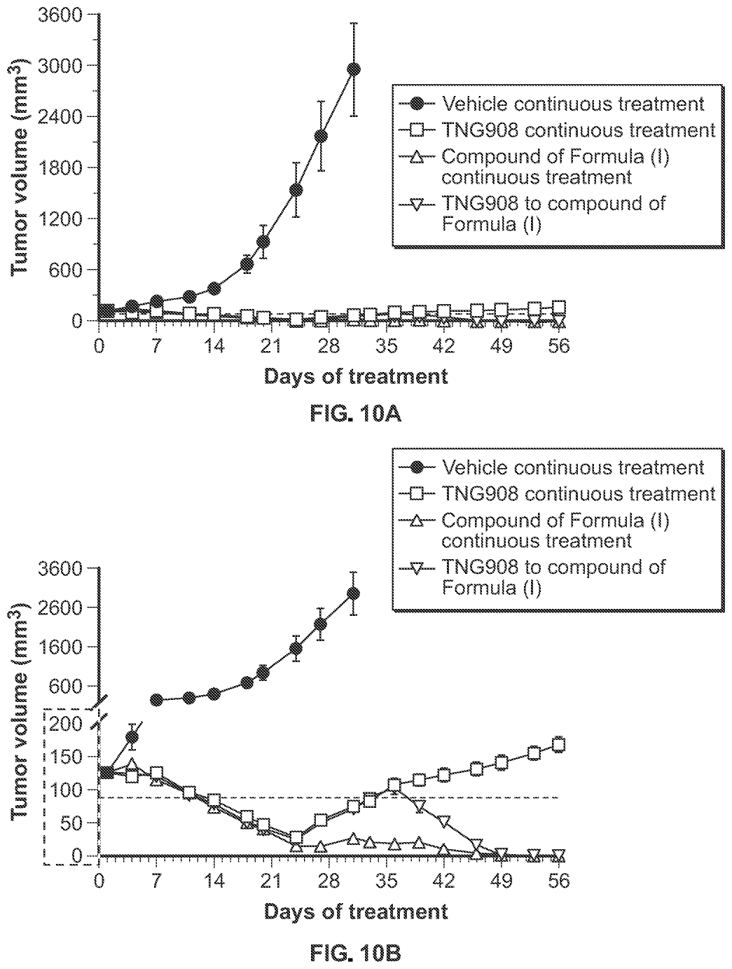

Briefly, mice were implanted with tumors derived from an MTAP-null OCI-LY19 cell line and were dosed as follows: group 1 (n=8) was dosed continuously with vehicle, group 2 (n=12) was dosed continuously with TNG908 (120 mg/kg BID), group 3 (n=10) was dosed continuously with the compound of Formula (I) (40 mg/kg BID), group 4 (n=12), was continuously dosed with TNG908 (120 mg/kg BID) and switched to the compound of Formula (I) 40 mg/kg BID when tumor volume recovered to approximate mean starting tumor volume. The data is shown in FIG. 10A and FIG. 10B as mean±SEM. Regression is defined as final mean tumor volume <30% initial mean tumor volume. The compound of Formula (I) is able to control tumors that appear to have developed resistance to TNG908.

### Example 16. The Compound of Formula (I) Synergizes with EGFR Inhibitors in MTAP-Null Xenograft Models

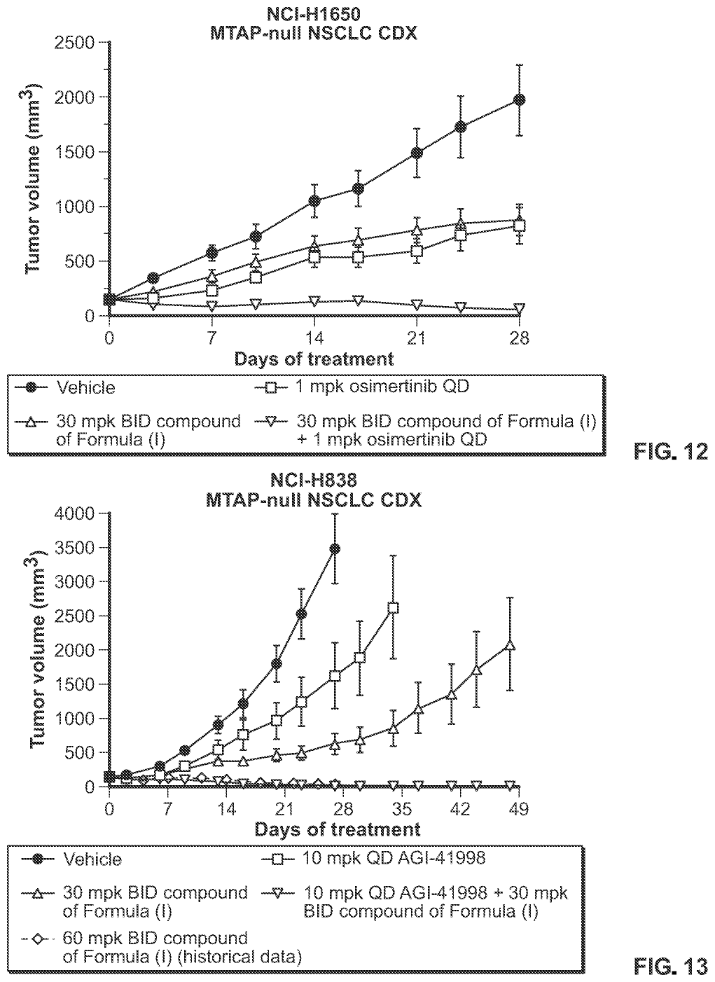

A MTAP-null NCI-H1650 NSCLC CDX model was used to determine the antitumor activity of a compound of Formula (I) with an EGFR inhibitor, osimertinib. Briefly the groups were treated for a minimum of 28 days and tumor-volumes were monitored for the indicated time period. Group 1 was treated with vehicle, group 2 was treated with osimertinib at 1 mg/kg QD, group 3 was treated with the compound of Formula (I) 30 mg/kg BID, and group 4 was treated with the compound of Formula (I) 30 mg/kg BID and osimertinib at 1 mg/kg QD. The results are shown in FIG. 12. Group 4 was the only group with a decrease in tumor volume, demonstrating synergy of the EGFR inhibitor and the compound of Formula (I).

### Example 17. The Compound of Formula (I) Synergizes with MAT2A Inhibitors in MTAP-Null Xenograft Models

An MTAP-null NCI-H838 NSCLC CDX model was used to determine the antitumor activity of a compound of Formula (I) with an methionine adenosyltransferase 2A (MAT2A) inhibitor, AGI-41998. Briefly the groups were treated for a minimum of 28 days and tumor-volumes were monitored for the indicated time period. Group 1 was treated with vehicle, group 2 was treated with AGI-41998 at 10 mg/kg QD, group 3 was treated with the compound of Formula (I) 30 mg/kg BID, group 4 was treated with the compound of Formula (I) 30 mg/kg BID and AGI-41998 at 10 mg/kg QD, and group 5 was treated with the compound of Formula (I) 60 mg/kg BID (historical data). The results are shown in FIG. 13. Groups 1-3 displayed increases in tumor volumes, while Groups 4 and 5 displayed decreases in tumor volume, demonstrating synergy of the MAT2A inhibitor and the compound of Formula (I).

### INCORPORATION BY REFERENCE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A crystalline form of N-(6-amino-5-ethylpyridin-3-yl)-2-((2R,5S)-5-methyl-2-(2-(1-methylpiperidin-4-yl)benzo[d]thiazol-5-yl)piperidin-1-yl)-2-oxoacetamide (a compound of formula (I)) 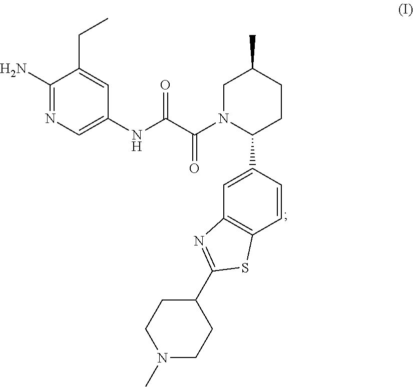 [SMILES: CCc1cc(NC(=O)C(=O)N2C[C@@H](C)CC[C@@H]2c2ccc3sc(C4CCN(C)CC4)nc3c2)cnc1N]
wherein the X-ray powder diffraction (XRPD) pattern of the crystalline form comprises one or more peaks at 2θ angles selected from 4.4±0.2, 9.6±0.2, 16.8±0.2, 18.6±0.2, 19.4±0.2, 20.9±0.2, 23.7±0.2 and 24.5±0.2 degrees.

2. The crystalline form of claim 1, wherein the X-ray powder diffraction (XRPD) pattern of the crystalline form comprises one or more peaks at 2θ angles selected from 4.4±0.2, 9.6±0.2, 16.8±0.2 and 24.5±0.2 degrees.

3. The crystalline form of claim 1, wherein the X-ray powder diffraction (XRPD) pattern of the crystalline form comprises three or more peaks at 2θ angles selected from 4.4±0.2, 9.6±0.2, 16.8±0.2 and 24.5±0.2 degrees.

4. The crystalline form of claim 1, wherein the X-ray powder diffraction (XRPD) pattern of the crystalline form comprises peaks at 2θ angles of 4.4±0.2, 9.6±0.2, 16.8±0.2, and 24.5±0.2 degrees.

5. The crystalline form of claim 1, wherein the X-ray powder diffraction (XRPD) pattern of the crystalline form comprises three or more peaks at 2θ angles selected from 4.4±0.2, 9.6±0.2, 16.8±0.2, 18.6±0.2, 19.4±0.2, 20.9±0.2, 23.7±0.2 and 24.5±0.2 degrees.

6. The crystalline form of claim 1, wherein the X-ray powder diffraction (XRPD) pattern of the crystalline form comprises peaks at 2θ angles of 4.4±0.2, 9.6±0.2, 16.8±0.2, and 24.5±0.2 degrees and additionally comprises at least one additional peak at 20 angles selected from 18.6±0.2, 19.4±0.2, 20.9±0.2 and 23.7±0.2.

7. The crystalline form of claim 1, wherein the X-ray powder diffraction (XRPD) pattern of the crystalline form comprises peaks at 2θ angles of 4.4±0.2, 9.6±0.2, 16.8±0.2, 18.6±0.2, 19.4±0.2, 20.9±0.2, 23.7±0.2 and 24.5±0.2 degrees.

8. A pharmaceutical composition comprising a compound of formula (I) or a pharmaceutically acceptable salt thereof and at least one pharmaceutically acceptable carrier, diluent, excipient or adjuvant.

9. A pharmaceutical composition comprising the crystalline form of the compound of Formula (I) of claim 1 and at least one pharmaceutically acceptable carrier, diluent, excipient or adjuvant.

10. The pharmaceutical composition of claim 9, wherein the composition comprises about 10% (w/w) of the crystalline form of the compound of formula (I).

11. A pharmaceutical composition comprising:
(a) the crystalline form of the compound of Formula (I) of claim 1
(b) a filler;
(c) a glidant;
(d) a disintegrant; and
(e) a lubricant.

12. The pharmaceutical composition of claim 11, wherein the composition comprises:
(a) about 10% (w/w) of the crystalline form of the compound of Formula (I);
(b) about 83.5% (w/w) of a filler;
(c) about 1.5% (w/w) of a glidant;
(d) about 4% (w/w) of a disintegrant; and
(e) about 1% (w/w) of a lubricant;
thereby totaling no more than 100% (w/w) of the composition.

13. A pharmaceutical composition comprising:
(a) the crystalline form of a compound of formula (I) of claim 1;
(b) an intragranular filler;
(c) an intragranular glidant;
(d) an intragranular disintegrant;
(e) an intragranular lubricant;
(f) an extragranular glidant;
(g) an extragranular disintegrant; and
(h) an extragranular lubricant.

14. The pharmaceutical composition of claim 13, wherein the composition comprises:
(a) about 2% (w/w) to about 20% (w/w) of the crystalline form of the compound of formula (I);
(b) about 50% (w/w) to about 90% (w/w) of an intragranular filler;
(c) about 0.75% (w/w) to about 1.25% (w/w) of an intragranular glidant;
(d) about 1% (w/w) to about 3% (w/w) of an intragranular disintegrant;
(e) about 0.25% (w/w) to about 0.75% (w/w) of an intragranular lubricant;
(f) about 0.25% (w/w) to about 0.75% (w/w) of an extragranular glidant;
(g) about 1% (w/w) to about 3% (w/w) of an extragranular disintegrant; and
(h) about 0.25% (w/w) to about 0.75% (w/w) of an extragranular lubricant;
thereby totaling no more than 100% (w/w) of the composition.

15. The pharmaceutical composition of claim 13, wherein the composition comprises:
(a) about 10% (w/w) of the crystalline form of the compound of formula (I);
(b) about 83.5% (w/w) of an intragranular filler;
(c) about 1% (w/w) of an intragranular glidant;
(d) about 2% (w/w) of an intragranular disintegrant;
(e) about 0.5% (w/w) of an intragranular lubricant;
(f) about 0.5% (w/w) of an extragranular glidant;
(g) about 2% (w/w) of an extragranular disintegrant;
(h) about 0.5% (w/w) of an extragranular lubricant;
thereby totaling no more than 100% (w/w) of the composition.

16. A dosage form comprising a pharmaceutical composition of claim 9.

17. The dosage form of claim 16, wherein the total weight of the pharmaceutical composition in the dosage form is about 100 mg to 500 mg.

18. The dosage form of claim 16, wherein the composition comprises about 10 mg to about 50 mg of a compound of formula (I).

19. The dosage form of claim 16, wherein the composition comprises about 10 mg or about 50 mg of the compound of formula (I).

20. A method for treating an MTAP-deficient and/or an MTA-accumulating disease in a subject in need thereof comprising administering to the subject a pharmaceutical composition of claim 9 containing a therapeutically effective amount of the compound of Formula (I).

21. The method of claim 20 wherein the disease is an MTAP-deficient and/or MTA-accumulating cancer.

22. A method of treating a cancer in a subject in need thereof comprising the steps of:
a) assessing the level of MTAP and/or MTA in a test sample obtained from said subject, wherein the MTA level can be assessed directly or indirectly;
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering the pharmaceutical composition of claim 9 containing an effective amount of the compound of formula a (I) to the subject identified in step b).

23. The method of claim 21 wherein the cancer is glioblastoma, malignant peripheral nerve sheath tumors (MPNST), esophageal cancer, bladder cancer, pancreatic cancer, mesothelioma, melanoma, non-small cell lung cancer, astrocytoma, undifferentiated pleomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

24. The method of claim 21, wherein the method further comprises administration of a second therapeutic agent.
